(19) United States
(12) Patent Application Publication
(10) Pub. No.: US 2012/0088759 A1
(43) Pub. Date: Apr. 12, 2012

(54) THIAZOLIDIN-4-ONE AND [1,3]-THIAZINAN-4-ONE COMPOUNDS AS OREXIN RECEPTOR ANTAGONISTS

(76) Inventors: Hamed Aissaoui, Pulversheim (FR); Christoph Boss, Allschwil (CH); Christine Brotschi, Allschwil (CH); Markus Gude, Allschwil (CH); John Gatfield, Basel (CH); Romain Siegrist, Allschwil (CH); Thierry Sifferlen, Wentzwiller (FR); Jodi T. Williams, Basel (CH)

(21) Appl. No.: 13/319,774

(22) PCT Filed: May 11, 2010
(86) PCT No.: PCT/IB2010/052067

§ 371 Date: Dec. 14, 2011

(30) Foreign Application Priority Data
May 12, 2009 (IB) PCT/IB2009/051950

(51) Int. Cl. A61K 31/54; C07D 277/34; C07D 417/12; A61K 31/506; A61K 31/4439; A61K 31/426; A61K 31/498; A61P 25/22; A61P 25/00; C07D 417/10; C07D 417/04; C07D 279/06; A61K 31/501; A61K 31/427; A61K 31/4709; A61K 31/541; A61P 25/20; A61P 25/18; C07D 277/14

(52) U.S. Cl. 514/227.2; 548/186; 548/183; 546/269.7; 544/298; 514/269; 514/342; 514/369; 514/249; 544/238; 548/181; 546/176; 544/353; 544/54; 514/252.05; 514/314

(57) ABSTRACT

The present invention relates to thiazolidin-4-one and [1,3]-thiazinan-4-one compounds of formula (I) wherein X, Y, R³ and R₄ are as described in the description, or pharmaceutically acceptable salts thereof, for the prevention or treatment of diseases related to the orexin system. The present invention also relates to novel thiazolidin-4-one compounds of formula (II) and their use as pharmaceuticals, pharmaceutical compositions containing one or more compounds of formula (II), and especially their use as orexin receptor antagonists.

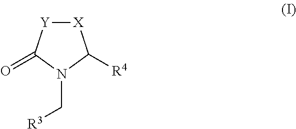

The present invention relates to thiazolidin-4-one and [1,3]-thiazinan-4-one compounds of formula (I), or pharmaceutically acceptable salts thereof, for the prevention or treatment of diseases related to the orexin system. The present invention also relates to novel thiazolidin-4-one compounds of formula (II) and their use as pharmaceuticals, pharmaceutical compositions containing one or more compounds of formula (II), and especially their use as orexin receptor antagonists. The invention also concerns related aspects including processes for the preparation of said compounds.

Orexins (orexin A or OX-A and orexin B or OX-B) are novel neuropeptides found in 1998 by two research groups, orexin A is a 33 amino acid peptide and orexin B is a 28 amino acid peptide (Sakurai T. et al., Cell, 1998, 92, 573-585). Orexins are produced in discrete neurons of the lateral hypothalamus and bind to the G-protein-coupled receptors (OX₁ and OX₂ receptors). The orexin-1 receptor (OX₁) is selective for OX-A, and the orexin-2 receptor (OX₂) is capable to bind OX-A as well as OX-B. Orexins are found to stimulate food consumption in rats suggesting a physiological role for these peptides as mediators in the central feedback mechanism that regulates feeding behaviour (Sakurai T. et al., Cell, 1998, 92, 573-585). On the other hand, it was also observed that orexins regulate states of sleep and wakefulness opening potentially novel therapeutic approaches to narcolepsy as well as insomnia and other sleep disorders (Chemelli R. M. et al., Cell, 1999, 98, 437-451). Furthermore, in vitro and in vivo evidence for a critical role of orexin signaling in the ventral tegmental area in neural plasticity relevant to addiction has been published (S. L. Borgland et al. Neuron, 2006, 49, 589-601).

Thus, orexin receptors may have numerous implications in pathologies as known from the literature, such as dysthymic, mood, psychotic and anxiety disorders; diabetes and appetite, taste, eating, or drinking disorders; hypothalamic diseases; disturbed biological and circadian rhythms; sleep disturbances associated with diseases such as neurological disorders, neuropathic pain and restless leg syndrome; insomnias related to psychiatric disorders; sleep apnea; narcolepsy; idiopathic insomnias; parasomnias; benign prostatic hypertrophy; all dementias and cognitive dysfunctions in the healthy population and in psychiatric and neurologic disorders; and other diseases related to general orexin system dysfunctions. The compound (2R)-2-{(1S)-6,7-dimethoxy-1-[2-(4-trifluoromethyl-phenyl)-ethyl]-3,4-dihydro-1H-isoquinolin-2-yl}-N-methyl-2-phenylacetamide (WO2005/118548) is currently in clinical development for primary insomnia.

In the rat, the compound has been shown for example to decrease alertness, characterized by decreases in both active wake and locomotion; and to dose-dependently increase the time spent in both REM and NREM sleep (F. Jenck et al., Nature Medicine 2007, 13, 150-155). The compound has also been shown to enhance memory function in a rat model (WO2007/105177) and is also active in a rat model of post-traumatic stress disorder (WO2009/047723).

The present invention provides thiazolidin-4-one and [1,3]-thiazinan-4-one compounds, which are non-peptide antagonists of human orexin receptors and, thus, of potential use in the treatment of diseases related to the orexin system, especially comprising all types of sleep disorders, of stress-related syndromes, of addictions (especially psychoactive substance use, abuse, seeking and reinstatement), of cognitive dysfunctions in the healthy population and in psychiatric and neurologic disorders, of eating or drinking disorders.

1) A first aspect of the invention relates to thiazolidin-4-one and [1,3]-thiazinan-4-one compounds, or pharmaceutically acceptable salts thereof, for the prevention or treatment of diseases related to the orexin system; wherein said compounds are compounds of the formula (I)

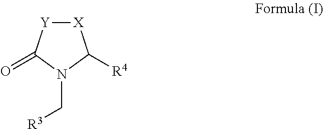

wherein
X represents S, S(O), or SO₂;
Y represents CH₂, CH₂CH₂, CHR¹, or CR¹R²; wherein
R¹ and R² independently represent (C₁₋₄)alkyl;
   R³ represents Ar¹ or Ar³—Z—Ar²-* wherein the asterisk indicates the bond that is attached to the rest of the molecule; wherein
Ar¹ represents aryl or heteroaryl, wherein the aryl or heteroaryl is independently unsubstituted, or mono-, di-, or tri-substituted, wherein the substituents are independently selected from the group consisting of (C₁₋₄)alkyl, (C₁₋₄)alkoxy, hydroxy-(C₁₋₄)alkoxy, halogen, (C₁₋₃)fluoroalkyl, (C₁₋₃)fluoroalkoxy, (C₁₋₃)fluoroalkyl-thio-, hydroxy-(C₁₋₄)alkoxy, (C₁₋₄)alkoxy-(C₁₋₄)alkoxy, and —NR⁵R⁶;
Ar² represents phenyl or 5- to 6-membered heteroaryl;
Z represents a bond, O, or —CH₂—O-* wherein the asterisk indicates the bond that is attached to Ar²;
Ar³ represents phenyl or 5- to 6-membered heteroaryl wherein the phenyl or 5- to 6-membered heteroaryl is independently unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably substituents are selected from (C₁₋₄)alkyl, (C₁₋₄)alkoxy, and (C₁₋₃)fluoroalkyl);
   R⁴ represents aryl or heteroaryl, wherein the aryl or heteroaryl is independently unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, hydroxy-(C₁₋₄)alkoxy, dihydroxy-(C₁₋₄)alkoxy, (C₁₋₄)alkoxy-(C₁₋₄)alkoxy, hydroxy, benzyloxy, and benzyl; and
   R⁵ and R⁶ together with the nitrogen to which they are attached form an azetidinyl, a pyrrolidinyl, or a piperidinyl ring (notably a pyrrolidinyl ring).

For avoidance of any doubt, if compounds are described for the prevention or treatment of certain diseases, such compounds are likewise suitable for use in the preparation of a medicament for the prevention or treatment of said diseases.

In this patent application, a bond interrupted by a wavy line shows the point of attachment of the radical drawn. For example, the radical drawn below

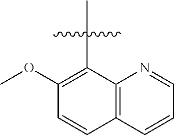

is the 7-methoxy-quinolin-8-yl group.

The term “halogen” means fluorine, chlorine, or bromine, preferably fluorine or chlorine.

The term “alkyl”, used alone or in combination, refers to a saturated straight or branched chain alkyl group containing one to four carbon atoms. The term “(C_{x-y})alkyl” (x and y each being an integer), refers to an alkyl group as defined before containing x to y carbon atoms. For example a (C₁₋₄)alkyl group contains from one to four carbon atoms. Examples of (C₁₋₄)alkyl groups are methyl, ethyl, propyl, isopropyl, n-butyl, isobutyl, sec.-butyl and tert.-butyl. Preferred are methyl and ethyl. Most preferred is methyl. For the substituents R¹ and R² preferred is methyl. For the substituent R¹⁵ representing a (C₁₋₄)alkyl group, preferred are ethyl and especially isopropyl.

The term “alkoxy”, used alone or in combination, refers to an alkyl-O— group wherein the alkyl group is as defined before. The term “(C_{x-y})alkoxy” (x and y each being an integer) refers to an alkoxy group as defined before containing x to y carbon atoms. For example a (C₁₋₄)alkoxy group means a group of the formula (C₁₋₄)alkyl-O— in which the term “(C₁₋₄)alkyl” has the previously given significance. Examples of (C₁₋₄)alkoxy groups are methoxy, ethoxy, n-propoxy, isopropoxy, n-butoxy, isobutoxy, sec.-butoxy and tert.-butoxy. Preferred are ethoxy and especially methoxy. For substituents of R⁴ preferred examples are ethoxy and methoxy. For substituents of Ar¹ representing a phenyl group preferred examples are methoxy, ethoxy and especially isopropoxy. For substituents of Ar³ a preferred example is methoxy.

The term “fluoroalkyl” refers to an alkyl group as defined before containing one to three carbon atoms in which one or more (and possibly all) hydrogen atoms have been replaced with fluorine. The term “(C_{x-y})fluoroalkyl” (x and y each being an integer) refers to a fluoroalkyl group as defined before containing x to y carbon atoms. For example a (C₁₋₃)fluoroalkyl group contains from one to three carbon atoms in which one to seven hydrogen atoms have been replaced with fluorine. Representative examples of fluoroalkyl groups include trifluoromethyl and 2,2,2-trifluoroethyl. Preferred are (C₁₋₃)fluoroalkyl groups such as trifluoromethyl. For the substituent R¹⁵ preferred examples are trifluoromethyl, difluoromethyl and 2,2,2-trifluoroethyl.

The term “fluoroalkoxy” refers to an alkoxy group as defined before containing one to three carbon atoms in which one or more (and possibly all) hydrogen atoms have been replaced with fluorine. The term “(C_{x-y})fluoroalkoxy” (x and y each being an integer) refers to a fluoroalkoxy group as defined before containing x to y carbon atoms. For example a (C₁₋₃)fluoroalkoxy group contains from one to three carbon atoms in which one to seven hydrogen atoms have been replaced with fluorine. Representative examples of fluoroalkoxy groups include trifluoromethoxy, difluoromethoxy and 2,2,2-trifluoroethoxy. Preferred are (C₁)fluoroalkoxy groups such as trifluoromethoxy and difluoromethoxy.

The term “aryl”, alone or in combination, means a phenyl or a naphthyl group. In addition, the term aryl also comprises phenyl rings fused to a 5- or 6-membered saturated or partially unsaturated non-aromatic ring optionally containing 1 to 2 oxygen atoms. Examples of aryl groups are phenyl, naphthyl, indanyl, tetrahydronaphthyl, benzo[1,3]dioxolyl, 2,3-dihydrobenzofuranyl, 2,3-dihydro-benzo[1,4]dioxinyl, chromanyl, and chromenyl. In a sub-embodiment, examples are phenyl, naphthyl, indanyl, benzo[1,3]dioxolyl, 2,3-dihydrobenzofuranyl, and 2,3-dihydro-benzo[1,4]dioxinyl. In another sub-embodiment an aryl group is a phenyl or naphthyl group, notably a phenyl group. The aryl group may be unsubstituted or substituted as explicitly defined. The sub-group wherein aryl groups are phenyl rings fused to a 5- or 6-membered saturated or partially unsaturated non-aromatic ring optionally containing 1 or 2 oxygen atoms is preferably unsubstituted, or mono-, or di-substituted wherein the substituents are independently selected from the group consisting of methyl, methoxy, and halogen.

For the substituent Ar¹ particular examples of aryl groups are phenyl, 2-naphthyl, 6-methoxy-naphthalen-2-yl, 1-naphthyl, 4-chlorophenyl, 3-chlorophenyl, 2-chlorophenyl, 3-fluorophenyl, 2-fluorophenyl, 4-methylphenyl, 3-methylphenyl, 2-methylphenyl, 3-fluoro-4-methylphenyl, 3,4-difluorophenyl, 3-chloro-4-fluorophenyl, 4-chloro-2-fluorophenyl, 3-chloro-6-fluorophenyl, 2-chloro-4-fluorophenyl, 2,4-dichlorophenyl, 3,4-difluoro-phenyl, 3,5-difluoro-phenyl, 2,5-difluoro-phenyl, 2,6-difluoro-phenyl, 3,4,5-trifluoro-phenyl, 4-methoxyphenyl, 3-methoxyphenyl, 2-methoxyphenyl, 2,3-dimethoxyphenyl, 2,4-dimethoxyphenyl, 3,4-dimethoxyphenyl, 2,4,6-trimethylphenyl, 4-ethoxyphenyl, 2-ethoxyphenyl, 4-isopropoxyphenyl, 4-trifluoromethylphenyl, 2-trifluoromethylphenyl, 4-tert.butyl-phenyl, 2,6-dimethoxyphenyl, 2,5-dimethoxyphenyl, 4-(2-hydroxy-ethoxy)-phenyl, 4-(2-methoxy-ethoxy)-phenyl, 2-difluoromethoxy-phenyl, 4-trifluoromethyl-sulfanyl-phenyl, 4-trifluoromethoxyphenyl, 3-trifluoromethoxyphenyl, 2-trifluoromethoxyphenyl, 3-fluoro-4-trifluoromethylphenyl, 4-(2,2,2-trifluoroethoxy)phenyl, 4-tert.-butoxyphenyl, benzo[1,3]dioxol-5-yl, 2,2-difluoro-benzo[1,3]dioxol-5-yl, 2,3-dihydrobenzofuran-5-yl, and 2,3-dihydro-benzo[1,4]dioxin-6-yl.

For the substituent R⁴ particular examples of aryl groups are phenyl, 2-methoxy-naphthalen-1-yl, 2,6-difluorophenyl, 2-methoxyphenyl, 2-ethoxyphenyl, 4-hydroxy-2-methoxyphenyl, 2-fluoro-6-methoxyphenyl, 2-chloro-6-methoxyphenyl, 2-methoxy-6-methylphenyl, 2-methoxy-5-methylphenyl, 2,6-dimethoxyphenyl, 2,5-dimethoxyphenyl, 3-chloro-2,6-dimethoxyphenyl, 2-chloro-4,6-dimethoxyphenyl, 2-fluoro-4,6-dimethoxyphenyl, 4-fluoro-2,6-dimethoxyphenyl, 2-ethoxy-6-methoxyphenyl, 2,6-diethoxyphenyl, 3-fluoro-2,6-dimethoxyphenyl, 2,6-dimethoxy-3-methylphenyl, 2,6-dimethoxy-4-methylphenyl, 2-(2-hydroxyethoxy)-6-methoxyphenyl, 2-(2-hydroxypropoxy)-6-methoxyphenyl, 2-(2,3-dihydroxypropoxy)-6-methoxyphenyl, 2-(2-methoxyethoxy)-6-methoxyphenyl, 2,3,6-trimethoxyphenyl, 2,4,6-trimethoxyphenyl, 2-trifluoromethylphenyl, 6-methoxy-indan-5-yl, benzo[1,3]dioxol-4-yl, 5-methoxy-benzo[1,3]dioxol-4-yl, 5-bromo-benzo[1,3]dioxol-4-yl, 2,2-dimethyl-2,3-dihydrobenzofuran-7-yl, 2,3-dihydro-benzo[1,4]dioxin-5-yl, 6-methoxy-2,3-dihydro-benzo[1,4]dioxin-5-yl, 3-benzyl-5-methoxyphenyl, and 4-benzyloxy-3-methoxyphenyl. In a sub-embodiment, examples are 2-ethoxyphenyl, 2,6-dimethoxyphenyl, 2-ethoxy-6-methoxyphenyl, 4-fluoro-2,6-dimethoxyphenyl, 2,6-dimethoxy-4-methylphenyl, 2-(2-hydroxyethoxy)-6-methoxyphenyl, 5-methoxy-benzo[1,3]dioxol-4-yl, and 6-methoxy-2,3-dihydro-benzo[1,4]dioxin-5-yl.

For the substituent Ar¹ examples of the particular sub-group of “phenyl rings fused to a 5- or 6-membered saturated or partially unsaturated non-aromatic ring optionally containing 1 to 2 oxygen atoms” are 2,2-difluoro-benzo[1,3]dioxol-5-yl, benzo[1,3]dioxol-5-yl, 2,3-dihydrobenzofuran-5-yl, and 2,3-dihydro-benzo[1,4]dioxin-6-yl.

For the substituent R⁴ examples of the particular sub-group of “phenyl rings fused to a 5- or 6-membered saturated or partially unsaturated non-aromatic ring optionally containing 1 to 2 oxygen atoms” are 6-methoxy-indan-5-yl, benzo[1,3]dioxol-4-yl, 5-methoxy-benzo[1,3]dioxol-4-yl, 5-bromo-benzo[1,3]dioxol-4-yl, 2,2-dimethyl-2,3-dihydrobenzofuran-7-yl, 2,3-dihydro-benzo[1,4]dioxin-5-yl, and 6-methoxy-2,3-dihydro-benzo[1,4]dioxin-5-yl (notably 5-methoxy-benzo[1,3]dioxol-4-yl, and 6-methoxy-2,3-dihydro-benzo[1,4]dioxin-5-yl).

The term “heteroaryl”, alone or in combination, means a 5- to 10-membered monocyclic or bicyclic aromatic ring containing 1 to a maximum of 3 heteroatoms independently selected from oxygen, nitrogen and sulfur. Examples of such heteroaryl groups are furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidyl, pyridazinyl, pyrazinyl, indolyl, isoindolyl, benzofuranyl, isobenzofuranyl, benzothiophenyl, indazolyl, benzimidazolyl, benzoxazolyl, benzisoxazolyl, benzothiazolyl, benzoisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidyl, imidazo[1,2-a]pyridyl, 1H-pyrrolo[3,2-b]pyridyl, 1H-pyrrolo[2,3-b]pyridyl, 4H-furo[3,2-b]pyrrolyl, pyrrolo[2,1-b]thiazolyl and imidazo[2,1-b]thiazolyl. In case the heteroaryl group is a 5- to 6-membered heteroaryl as used for the generic groups Ar² and Ar³, particular examples are furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidyl, pyridazinyl, and pyrazinyl; notably pyrrolyl, pyrazolyl, pyridyl, pyrimidyl, pyridazinyl, and pyrazinyl. The heteroaryl group may be unsubstituted or substituted as explicitly defined.

For the substituent Ar¹ particular examples of heteroaryl groups are thiophenyl, pyrrolyl, pyridazinyl, pyridinyl, benzo[b]thiophenyl, benzofuranyl, benzimidazolyl, benzothiazolyl, indolyl, benzotriazolyl, quinoxalinyl, and quinolinyl, which are unsubstituted or substituted as explicitly defined; notably thiophen-2-yl, 1-methyl-pyrrol-2-yl, 6-ethoxy-pyridazin-3-yl, pyridin-2-yl, pyridin-4-yl, 2-ethoxy-pyridin-5-yl, 5-ethoxy-pyridin-2-yl, benzo[b]thiophen-2-yl, benzo[b]thiophen-3-yl, benzo[b]thiophen-5-yl, benzofuran-2-yl, 1H-benzimidazol-2-yl, 1-methyl-1H-benzimidazol-2-yl, benzothiazol-2-yl, 1-methyl-1H-indol-6-yl, 1H-indol-2-yl, 1-methyl-1H-indol-2-yl, 1H-indol-3-yl, 1-methyl-1H-benzotriazol-5-yl, quinoxalin-2-yl, quinoxalin-6-yl, quinolin-2-yl, quinolin-6-yl, and quinolin-7-yl.

For the substituent R⁴ particular examples of heteroaryl groups are pyrazolyl, thiazolyl, pyrimidinyl, pyridinyl, indazolyl, indolyl, benzo[d]isoxazolyl, benzoxazolyl, and quinolinyl which are unsubstituted or substituted as explicitly defined; notably 1-methyl-pyrazol-5-yl, thiazol-2-yl, 4,6-dimethoxypyrimidin-5-yl, 2-methoxypyridin-3-yl, 2-ethoxypyridin-3-yl, 2,4-dimethoxypyridin-3-yl, 6-methoxypyridin-2-yl, 3,5-dimethoxypyridin-4-yl, 1-methyl-1H-indazol-3-yl, 1H-indol-2-yl, 1-methyl-1H-indol-3-yl, 6-methoxy-3-methyl-benzo[d]isoxazol-7-yl, 6-methoxy-2-methyl-benzoxazol-7-yl, 7-methoxy-quinolin-8-yl, and 6-methoxy-quinolin-5-yl. Particular examples are 2,4-dimethoxypyridin-3-yl, 3,5-dimethoxypyridin-4-yl, 6-methoxy-3-methyl-benzo[d]isoxazol-7-yl, 6-methoxy-2-methyl-benzoxazol-7-yl, and 7-methoxy-quinolin-8-yl.

For the substituent Ar³ particular examples of the particular sub-group of “5- to 6-membered heteroaryl” are pyrrolyl, pyrazolyl, pyridinyl, pyrimidinyl, pyridazinyl, and pyrazinyl which unsubstituted or are substituted as explicitly defined; notably pyrrol-1-yl, pyrazol-1-yl, pyridin-2-yl, pyridin-3-yl, 3-methyl-pyridin-2-yl, 4-methyl-pyridin-2-yl, 5-methyl-pyridin-2-yl, 6-methyl-pyridin-2-yl, 5-methoxy-pyridin-2-yl, 6-methoxy-pyridin-2-yl, 6-trifluoromethyl-pyridin-2-yl, pyrimidin-5-yl, 6-methyl-pyridazin-3-yl, and pyrazin-2-yl. Preferred (for the substituent Ar³, and mutatis mutandis also for the substituent R¹⁵) are 6-membered heteroaryl of the above listed groups, notably pyridin-2-yl, pyridin-3-yl, 4-methyl-pyridin-2-yl, and pyrazin-2-yl.

For the substituent Ar² the phenyl or 5- to 6-membered heteroaryl are preferably substituted by Z and the rest of the molecule in a para (such as phenyl-1,4-diyl) arrangement (for phenyl or 6-membered heteroaryl) or in a 1,3-arrangement (for 5-membered heteroaryl). A particular example of Ar² representing a 5- to 6-membered heteroaryl groups is pyridyl, notably pyridin-2,5-diyl (wherein Z may be attached in either position 2 or 5).

The term “fluoroalkyl-thio” refers to an alkyl group as defined before containing one to three carbon atoms in which one or more (and possibly all) hydrogen atoms have been replaced with fluorine, said group being attached to the rest of the molecule via a sulfur atom. The term “(C_{x-y})fluoroalkyl-thio” (x and y each being an integer) refers to a fluoroalkyl-thio group as defined before containing x to y carbon atoms. For example a (C₁₋₃)fluoroalkyl-thio group contains from one to three carbon atoms in which one to seven hydrogen atoms have been replaced with fluorine. A representative example of a fluoroalkyl-thio group is trifluoromethyl-sulfanyl (F₃C—S—).

The term “hydroxy-(C₁₋₄)alkoxy” refers to an alkoxy group as defined before containing one to four carbon atoms in which one hydrogen atom has been replaced with hydroxy. Representative examples of hydroxy-(C₁₋₄)alkoxy groups are 2-hydroxy-ethoxy and 2-hydroxy-propoxy (notably 2-hydroxy-ethoxy).

The term “dihydroxy-(C₁₋₄)alkoxy” refers to refers to an alkoxy group as defined before containing one to four carbon atoms in which two hydrogen atoms have been replaced with hydroxy. A representative example of a dihydroxy-(C₁₋₄)alkoxy group is 2,3-dihydroxy-propoxy.

The term “(C₁₋₄)alkoxy-(C₁₋₄)alkoxy” refers to an alkoxy group as defined before containing one to four carbon atoms in which one hydrogen atom has been replaced with a (C₁₋₄)alkoxy group as defined before. A representative example of a (C₁₋₄)alkoxy-(C₁₋₄)alkoxy group is 2-methoxy-ethoxy.

Further embodiments of the invention are presented hereafter:

2) A further embodiment of the invention relates to compounds according to embodiment 1), or pharmaceutically acceptable salts thereof, for the prevention or treatment of diseases related to the orexin system; which are also compounds of formula (I_{E1}) wherein the stereocenter at position 2 of the heterocyclic moiety is in absolute (S)-configuration:

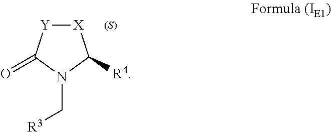

3) A further embodiment of the invention relates to compounds according to embodiment 1), or pharmaceutically acceptable salts thereof, for the prevention or treatment of diseases related to the orexin system; which are also compounds of formula (I_{E2}) wherein the stereocenter at position 2 of the heterocyclic moiety is in absolute (R)-configuration:

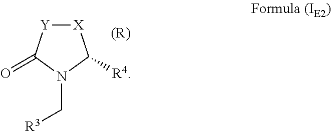

4) A further embodiment of the invention relates to compounds according to any one of embodiments 1) to 3), or pharmaceutically acceptable salts thereof, for the prevention or treatment of diseases related to the orexin system; wherein X represents S.

5) A further embodiment of the invention relates to compounds according to any one of embodiments 1) to 3), or pharmaceutically acceptable salts thereof, for the prevention or treatment of diseases related to the orexin system; wherein X represents S(O), or SO₂ (notably SO₂).

6) A further embodiment of the invention relates to compounds according to any one of embodiments 1) to 5), or pharmaceutically acceptable salts thereof, for the prevention or treatment of diseases related to the orexin system; wherein Y represents CH₂, CHR¹, or CR¹R²; wherein each group forms a particular sub-embodiment.

7) A further embodiment of the invention relates to compounds according to any one of embodiments 1) to 5), or pharmaceutically acceptable salts thereof, for the prevention or treatment of diseases related to the orexin system; wherein Y represents CH₂CH₂.

8) A further embodiment of the invention relates to compounds according to any one of embodiments 1) to 6), or pharmaceutically acceptable salts thereof, for the prevention or treatment of diseases related to the orexin system; wherein, if present, R¹ represents methyl and, if present, R² represents methyl.

9) A further embodiment of the invention relates to compounds according to any one of embodiments 1) to 8), or pharmaceutically acceptable salts thereof, for the prevention or treatment of diseases related to the orexin system; wherein R³ represents Ar¹.

10) A further embodiment of the invention relates to compounds according to any one of embodiments 1) to 8), or pharmaceutically acceptable salts thereof, for the prevention or treatment of diseases related to the orexin system; wherein R³ represents Ar³—Z—Ar²-* wherein the asterisk indicates the bond that is attached to the rest of the molecule.

11) A further embodiment of the invention relates to compounds according to any one of embodiments 1) to 9), or pharmaceutically acceptable salts thereof, for the prevention or treatment of diseases related to the orexin system; wherein
Ar¹ represents aryl which is unsubstituted, or mono-, di-, or tri-substituted (notably mono-, or di-substituted), wherein the substituents are independently selected from the group consisting of (C₁₋₄)alkyl, (C₁₋₄)alkoxy, hydroxy-(C₁₋₄)alkoxy, halogen, (C₁₋₃)fluoroalkyl, (C₁₋₃)fluoroalkoxy, (C₁₋₃)fluoroalkyl-thio-, hydroxy-(C₁₋₄)alkoxy, (C₁₋₄)alkoxy-(C₁₋₄)alkoxy, and —NR⁵R⁶ (notably (C₁₋₄)alkyl, (C₁₋₄)alkoxy, halogen, (C₁₋₃)fluoroalkyl, and (C₁₋₃)fluoroalkoxy); or
Ar¹ represents heteroaryl which is unsubstituted, or mono-, di-, or tri-substituted (notably mono-, or di-substituted), wherein the substituents are independently selected from the group consisting of (C₁₋₄)alkyl, (C₁₋₄)alkoxy and (C₁₋₃)fluoroalkyl (notably (C₁₋₄)alkyl, and (C₁₋₄)alkoxy).

12) A further embodiment of the invention relates to compounds according to any one of embodiments 1) to 9) or 11), or pharmaceutically acceptable salts thereof, for the prevention or treatment of diseases related to the orexin system; wherein
Ar¹ represents aryl which is unsubstituted, or mono-, di-, or tri-substituted (notably mono-, or di-substituted), wherein the substituents are independently selected from the group consisting of (C₁₋₄)alkyl, (C₁₋₄)alkoxy, halogen, (C₁₋₃)fluoroalkyl, and (C₁₋₃)fluoroalkoxy.

13) A further embodiment of the invention relates to compounds according to any one of embodiments 1) to 9), 11) or 12), or pharmaceutically acceptable salts thereof, for the prevention or treatment of diseases related to the orexin system; wherein, in case Ar¹ represents aryl, said aryl is
phenyl which is unsubstituted, or mono-, di-, or tri-substituted (notably mono-, or di-substituted), wherein the substituents are independently selected from the group consisting of (C₁₋₄)alkyl, (C₁₋₄)alkoxy, hydroxy-(C₁₋₄)alkoxy, halogen, (C₁₋₃)fluoroalkyl, (C₁₋₃)fluoroalkoxy, (C₁₋₃)fluoroalkyl-thio-, hydroxy-(C₁₋₄)alkoxy, (C₁₋₄)alkoxy-(C₁₋₄)alkoxy, and —NR⁵R⁶ (notably (C₁₋₄)alkyl, (C₁₋₄)alkoxy, halogen, (C₁₋₃)fluoroalkyl, and (C₁₋₃)fluoroalkoxy); or
naphthyl (notably 2-naphthyl) which is unsubstituted, or mono-, or di-substituted (notably unsubstituted or mono-substituted), wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably (C₁₋₄)alkoxy); or
a phenyl ring fused to a 5- or 6-membered saturated or partially unsaturated non-aromatic ring optionally containing 1 to 2 oxygen atoms (notably an indanyl, a benzo[1,3]dioxolyl, a 2,3-dihydrobenzofuranyl, or a 2,3-dihydro-benzo[1,4]dioxinyl group) which is (preferably) unsubstituted, or mono-, or di-substituted wherein the substituents are independently selected from the group consisting of methyl, methoxy, and halogen.

14) A further embodiment of the invention relates to compounds according to any one of embodiments 1) to 9) or 11), or pharmaceutically acceptable salts thereof, for the prevention or treatment of diseases related to the orexin system; wherein
Ar¹ represents heteroaryl which is unsubstituted, or mono-, or di-substituted, wherein the substituents are independently selected from the group consisting of (C₁₋₄)alkyl, (C₁₋₄)alkoxy and (C₁₋₃)fluoroalkyl (notably (C₁₋₄)alkyl, and (C₁₋₄)alkoxy).

15) A further embodiment of the invention relates to compounds according to any one of embodiments 1) to 8) or 10) to 14), or pharmaceutically acceptable salts thereof, for the prevention or treatment of diseases related to the orexin system; wherein Ar² represents phenyl (notably phenyl-1,4-diyl).

16) A further embodiment of the invention relates to compounds according to any one of embodiments 1) to 8) or 10) to 15), or pharmaceutically acceptable salts thereof, for the prevention or treatment of diseases related to the orexin system; wherein Z represents O.

17) A further embodiment of the invention relates to compounds according to any one of embodiments 1) to 8) or 10) to 15), or pharmaceutically acceptable salts thereof, for the prevention or treatment of diseases related to the orexin system; wherein Z represents a bond.

18) A further embodiment of the invention relates to compounds according to any one of embodiments 1) to 8) or 10) to 17), or pharmaceutically acceptable salts thereof, for the prevention or treatment of diseases related to the orexin system; wherein
Ar³ represents phenyl which is unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably Ar³ represents unsubstituted phenyl); or
Ar³ represents 5- to 6-membered (notably 6-membered) heteroaryl which is unsubstituted, or mono-, di-, or tri-substituted (notably unsubstituted or mono-substituted), wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably substituents are selected from (C₁₋₄)alkyl, (C₁₋₄)alkoxy, and (C₁₋₃)fluoroalkyl).

19) A further embodiment of the invention relates to compounds according to any one of embodiments 11) to 8) or 10) to 18), or pharmaceutically acceptable salts thereof, for the prevention or treatment of diseases related to the orexin system; wherein
Ar³ represents 5- to 6-membered (notably 6-membered) heteroaryl which is unsubstituted, or mono-, di-, or tri-substituted (notably unsubstituted or mono-substituted), wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably substituents are selected from (C₁₋₄)alkyl, (C₁₋₄)alkoxy, and (C₁₋₃)fluoroalkyl).

20) A further embodiment of the invention relates to compounds according to any one of embodiments 1) to 8) or 10) to 18), or pharmaceutically acceptable salts thereof, for the prevention or treatment of diseases related to the orexin system; wherein
Ar³ represents phenyl which is unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably Ar³ represents unsubstituted phenyl).

21) A further embodiment of the invention relates to compounds according to any one of embodiments 1) to 8) or 10) to 18), wherein R³ represents
phenyl which is mono-, or di-substituted, wherein one substituent is (C₁₋₄)alkoxy, or (C₁₋₃)fluoroalkoxy in position 4 of said phenyl and the other (if present) is selected from the group consisting of (C₁₋₄)alkyl, (C₁₋₄)alkoxy, halogen, (C₁₋₃)fluoroalkyl, and (C₁₋₃)fluoroalkoxy (notably halogen); or
2-naphthyl which is unsubstituted or mono-substituted in position 6, wherein the substituent is (C₁₋₄)alkoxy, or (C₁₋₃)fluoroalkoxy; or
2-quinolinyl which is unsubstituted or mono-substituted in position 6, wherein the substituent is (C₁₋₄)alkoxy, or (C₁₋₃)fluoroalkoxy; or
Ar³—Z—Ar²-* wherein the asterisk indicates the bond that is attached to the rest of the molecule; wherein
   Ar² represents phenyl or 6-membered heteroaryl which are substituted by Z and the rest of the molecule in a para arrangement,
   Z represents O, and
   Ar³ represents phenyl or 5- to 6-membered heteroaryl wherein the phenyl or 5- to 6-membered heteroaryl is independently unsubstituted or mono-substituted, wherein the substituent is independently selected from the group consisting of (C₁₋₄)alkyl, (C₁₋₄)alkoxy, halogen, (C₁₋₃)fluoroalkyl, and (C₁₋₃)fluoroalkoxy (notably Ar³ represents unsubstituted phenyl or 5- to 6-membered heteroaryl).

22) A further embodiment of the invention relates to compounds according to any one of embodiments 1) to 21), or pharmaceutically acceptable salts thereof, for the prevention or treatment of diseases related to the orexin system; wherein
R⁴ represents aryl which is unsubstituted, or mono-, di-, or tri-substituted (notably mono-, or di-substituted), wherein the substituents are independently selected from the group consisting of (C₁₋₄)alkyl, (C₁₋₄)alkoxy, halogen, (C₁₋₃)fluoroalkyl, (C₁₋₃)fluoroalkoxy, hydroxy-(C₁₋₄)alkoxy, dihydroxy-(C₁₋₄)alkoxy, (C₁₋₄)alkoxy-(C₁₋₄)alkoxy, hydroxy, benzyloxy, and benzyl (notably substituents are selected from (C₁₋₄)alkyl, (C₁₋₄)alkoxy, halogen, (C₁₋₃)fluoroalkyl, (C₁₋₃)fluoroalkoxy, and hydroxy-(C₁₋₄)alkoxy); or
R⁴ represents heteroaryl which is unsubstituted, or mono-, di-, or tri-substituted (notably mono-, or di-substituted), wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably substituents are selected from (C₁₋₄)alkyl, and (C₁₋₄)alkoxy).

23) A further embodiment of the invention relates to compounds according to any one of embodiments 1) to 22), or pharmaceutically acceptable salts thereof, for the prevention or treatment of diseases related to the orexin system; wherein
R⁴ represents aryl which is unsubstituted, or mono-, di-, or tri-substituted (notably mono-, or di-substituted), wherein the substituents are independently selected from the group consisting of (C₁₋₄)alkyl, (C₁₋₄)alkoxy, halogen, (C₁₋₃)fluoroalkyl, (C₁₋₃)fluoroalkoxy, hydroxy-(C₁₋₄)alkoxy, dihydroxy-(C₁₋₄)alkoxy, (C₁₋₄)alkoxy-(C₁₋₄)alkoxy, hydroxy, benzyloxy, and benzyl (notably substituents are selected from (C₁₋₄)alkyl, (C₁₋₄)alkoxy, halogen, (C₁₋₃)fluoroalkyl, (C₁₋₃)fluoroalkoxy, and hydroxy-(C₁₋₄)alkoxy).

24) A further embodiment of the invention relates to compounds according to any one of embodiments 1) to 22), or pharmaceutically acceptable salts thereof, for the prevention or treatment of diseases related to the orexin system; wherein
R⁴ represents heteroaryl which is unsubstituted, or mono-, di-, or tri-substituted (notably mono-, or di-substituted), wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably substituents are selected from (C₁₋₄)alkyl, and (C₁₋₄)alkoxy).

25) A further embodiment of the invention relates to compounds according to any one of embodiments 1) to 23), or pharmaceutically acceptable salts thereof, for the prevention or treatment of diseases related to the orexin system; wherein, in case R⁴ represents aryl, said aryl is
phenyl which is unsubstituted, or mono-, di-, or tri-substituted (notably mono-, or di-substituted), wherein the substituents are independently selected from the group consisting of (C₁₋₄)alkyl, (C₁₋₄)alkoxy, halogen, (C₁₋₃)fluoroalkyl, (C₁₋₃)fluoroalkoxy, hydroxy-(C₁₋₄)alkoxy, dihydroxy-(C₁₋₄)alkoxy, (C₁₋₄)alkoxy-(C₁₋₄)alkoxy, hydroxy, benzyloxy, and benzyl (notably substituents are selected from (C₁₋₄)alkyl, (C₁₋₄)alkoxy, halogen, (C₁₋₃)fluoroalkyl, (C₁₋₃)fluoroalkoxy, and hydroxy-(C₁₋₄)alkoxy); or
naphthyl (notably 1-naphthyl) which is unsubstituted, or mono-, or di-substituted (notably mono-substituted), wherein the substituents are independently selected from the group consisting of (C₁₋₄)alkyl, (C₁₋₄)alkoxy, halogen, (C₁₋₃)fluoroalkyl, and (C₁₋₃)fluoroalkoxy (notably (C₁₋₄)alkoxy); or
a phenyl ring fused to a 5- or 6-membered saturated or partially unsaturated non-aromatic ring optionally containing 1 to 2 oxygen atoms (notably a 2,3-dihydro-benzo[1,4]dioxinyl group) which is unsubstituted, or mono-, or di-substituted (notably mono-substituted) wherein the substituents are independently selected from the group consisting of methyl, methoxy, and halogen (notably methoxy).

26) A further embodiment of the invention relates to compounds according to any one of embodiments 1) to 25), or pharmaceutically acceptable salts thereof, for the prevention or treatment of diseases related to the orexin system; wherein R⁴ is at least mono-substituted, wherein said substituent is attached in ortho position to the point of attachment of R⁴ to the rest of the molecule; wherein, in case R⁴ represents a phenyl group, said substituent is preferably selected from the group consisting of (C₁₋₄)alkoxy, (C₁₋₃)fluoroalkoxy, and hydroxy-(C₁₋₄)alkoxy (notably (C₁₋₄)alkoxy); and, in case R⁴ represents a group different from phenyl, said substituent is preferably methoxy.

27) The invention further relates to novel thiazolidin-4-one compounds of formula (I) according to embodiment 1) which are also compounds of formula (II)

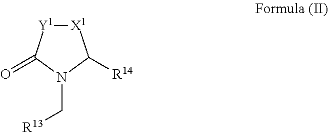

wherein
X¹ represents S or SO₂;
Y¹ represents CH₂ or CH(CH₃);
R¹³ represents

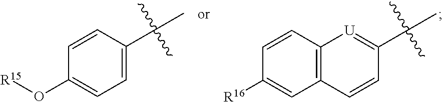

wherein
R¹⁵ represents (C₁₋₄)alkyl or (C₁₋₃)fluoroalkyl; or R¹⁵ represents phenyl or 5- to 6-membered (notably 6-membered) heteroaryl, wherein the phenyl or 5- to 6-membered heteroaryl is independently unsubstituted, or mono-substituted, wherein the substituent is selected from the group consisting of (C₁₋₄)alkyl, and (C₁₋₄)alkoxy;
U represents CH or N; and
R¹⁶ represents hydrogen or methoxy;
   R¹⁴ represents a group selected from the group consisting of:

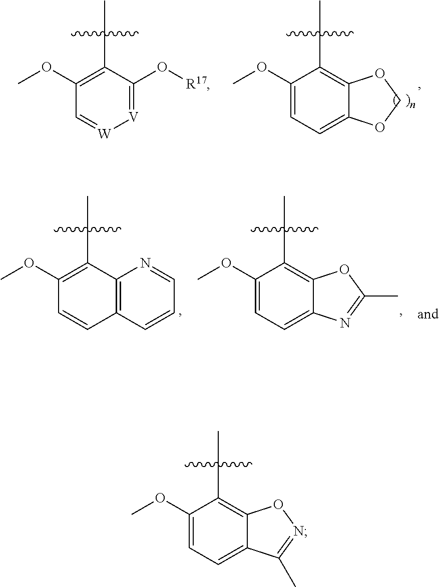

wherein
V represents CH and W represents CR¹⁸ or N; or V represents N and W represents CH;
R¹⁷ represents methyl, ethyl or hydroxyethyl;
R¹⁸ represents hydrogen, methyl or fluoro; and
n represents the integer 1 or 2;
   wherein characteristics described for compounds of formula (I), notably those described in embodiments 2) to 5), apply mutatis mutandis also to compounds of formula (II).

28) A further embodiment of the invention relates to compounds of formula (II) according to embodiment 27); wherein, R¹³ represents

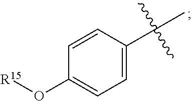

wherein
R¹⁵ represents (C₁₋₄)alkyl or (C₁₋₃)fluoroalkyl; or
R¹⁵ represents phenyl or 5- to 6-membered (notably 6-membered) heteroaryl, wherein the phenyl or 5- to 6-membered heteroaryl is independently unsubstituted, or mono-substituted, wherein the substituent is selected from the group consisting of (C₁₋₄)alkyl, and (C₁₋₄)alkoxy;
   wherein each R¹⁵ constitutes a particular sub-embodiment.

29) A further embodiment of the invention relates to compounds of formula (II) according to embodiment 27); wherein, R¹³ represents

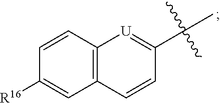

wherein
U represents CH or N; and
R¹⁶ represents hydrogen or methoxy;
   wherein each combination of U and R¹⁶ constitutes a particular sub-embodiment (notably: U is CH and R¹⁶ is methoxy).

30) A further embodiment of the invention relates to compounds of formula (II) according to any one of embodiments 27) to 29); wherein, R¹⁴ represents

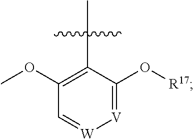

wherein
V represents CH and W represents CR¹⁸ or N; or
V represents N and W represents CH;
R¹⁷ represents methyl, ethyl or hydroxyethyl; and
R¹⁸ represents hydrogen, methyl or fluoro;
   wherein each combination of V, W, R¹⁷ and R¹⁸ constitutes a particular sub-embodiment.

31) A further embodiment of the invention relates to compounds of formula (II) according to embodiment 30); wherein V represents CH; W represents CR¹⁸; R¹⁷ represents methyl; and R¹⁸ represents hydrogen, or fluoro (especially hydrogen);

32) A further embodiment of the invention relates to compounds of formula (II) according to any one of embodiments 27) to 29); wherein,

R¹⁴ represents

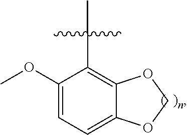

wherein n represents the integer 1 or 2.

33) A further embodiment of the invention relates to compounds of formula (II) according to any one of embodiments 27) to 29); wherein,

R¹⁴ represents a group selected from the group consisting of:

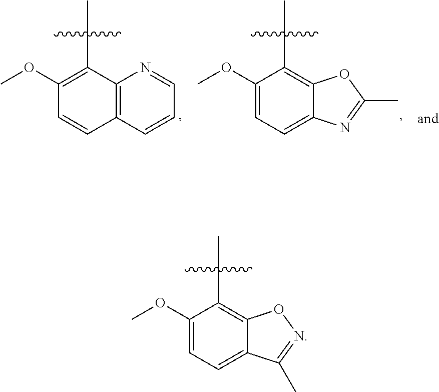

34) A further embodiment of the invention relates to compounds of formula (II) according to any one of embodiments 27) to 33); wherein, Y¹ represents CH₂.

35) A further embodiment of the invention relates to compounds of formula (II) according to any one of embodiments 27) to 33); wherein, Y¹ represents CH(CH₃).

36) A further embodiment of the invention relates to compounds according to any one of embodiments 27) to 35), or pharmaceutically acceptable salts thereof, for the prevention or treatment of diseases related to the orexin system.

The compounds of formulae (I) and (II) may contain one or more stereogenic or asymmetric centers, such as one or more asymmetric carbon atoms. The compounds of formulae (I) and (II) may thus be present as mixtures of stereoisomers or preferably as pure stereoisomers. Mixtures of stereoisomers may be separated in a manner known to a person skilled in the art.

Where the plural form is used for compounds, salts, pharmaceutical compositions, diseases and the like, this is intended to mean also a single compound, salt, or the like.

Any reference to compounds of formula (I) is to be understood as referring also to the pharmaceutically acceptable salts of such compounds, as appropriate and expedient. Any reference to compounds of formula (II) is to be understood as referring also to the salts (and especially the pharmaceutically acceptable salts) of such compounds, as appropriate and expedient.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

37) Another embodiment relates to compounds of formula (I) which are also compounds of formula (II) according to embodiment 27) selected from the group consisting of:
2-(2,6-Dimethoxy-phenyl)-3-(4-methoxy-benzyl)-thiazolidin-4-one;
2-(2,6-Dimethoxy-phenyl)-3-(4-trifluoromethoxy-benzyl)-thiazolidin-4-one;
3-(4-tert-Butoxy-benzyl)-2-(2,6-dimethoxy-phenyl)-thiazolidin-4-one;
3-(4-Difluoromethoxy-benzyl)-2-(2,6-dimethoxy-phenyl)-thiazolidin-4-one;
2-(2,6-Dimethoxy-phenyl)-3-(4-isopropoxy-benzyl)-thiazolidin-4-one;
2-(2,6-Dimethoxy-phenyl)-3-(4-ethoxy-benzyl)-thiazolidin-4-one;
2-(2,6-Dimethoxy-phenyl)-3-(4-phenoxy-benzyl)-thiazolidin-4-one;
2-(2,6-Dimethoxy-phenyl)-3-naphthalen-2-ylmethyl-thiazolidin-4-one;
3-(4-Ethoxy-benzyl)-2-(6-methoxy-2,3-dihydro-benzo[1,4]dioxin-5-yl)-thiazolidin-4-one;
2-(6-Methoxy-2,3-dihydro-benzo[1,4]dioxin-5-yl)-3-(4-trifluoromethoxy-benzyl)-thiazolidin-4-one;
3-(4-Ethoxy-benzyl)-2-(5-methoxy-benzo[1,3]dioxol-4-yl)-thiazolidin-4-one;
2-(5-Methoxy-benzo[1,3]dioxol-4-yl)-3-(4-trifluoromethoxy-benzyl)-thiazolidin-4-one;
2-(2,6-Dimethoxy-phenyl)-3-(4-ethoxy-benzyl)-5-methyl-thiazolidin-4-one;
2-(2,6-Dimethoxy-phenyl)-5-methyl-3-naphthalen-2-ylmethyl-thiazolidin-4-one;
3-(4-Ethoxy-benzyl)-2-(2-ethoxy-6-methoxy-phenyl)-thiazolidin-4-one;
2-(2-Ethoxy-6-methoxy-phenyl)-3-(4-trifluoromethoxy-benzyl)-thiazolidin-4-one;
2-(2,6-Dimethoxy-phenyl)-5-methyl-3-(4-trifluoromethoxy-benzyl)-thiazolidin-4-one;
2-(2,6-Dimethoxy-phenyl)-3-(4-(2,2,2-trifluoro-ethoxy)-benzyl)-thiazolidin-4-one;
3-(4-Ethoxy-benzyl)-2-(6-methoxy-3-methyl-benzo[d]isoxazol-7-yl)-thiazolidin-4-one;
3-(4-Ethoxy-benzyl)-2-(6-methoxy-2-methyl-benzooxazol-7-yl)-thiazolidin-4-one;
3-(4-Ethoxy-benzyl)-2-(7-methoxy-quinolin-8-yl)-thiazolidin-4-one;
2-(6-Methoxy-3-methyl-benzo[d]isoxazol-7-yl)-3-(4-trifluoromethoxy-benzyl)-thiazolidin-4-one;
2-(6-Methoxy-2-methyl-benzooxazol-7-yl)-3-(4-trifluoromethoxy-benzyl)-thiazolidin-4-one;
2-(7-Methoxy-quinolin-8-yl)-3-(4-trifluoromethoxy-benzyl)-thiazolidin-4-one;
2-(2,4-Dimethoxy-pyridin-3-yl)-3-(4-trifluoromethoxy-benzyl)-thiazolidin-4-one;
2-(2,4-Dimethoxy-pyridin-3-yl)-3-(4-ethoxy-benzyl)-thiazolidin-4-one;
2-(2,6-Dimethoxy-phenyl)-3-quinolin-2-ylmethyl-thiazolidin-4-one;
2-(3,5-Dimethoxy-pyridin-4-yl)-3-(4-trifluoromethoxy-benzyl)-thiazolidin-4-one;
2-(3,5-Dimethoxy-pyridin-4-yl)-3-(4-ethoxy-benzyl)-thiazolidin-4-one;
2-(2,6-Dimethoxy-phenyl)-5-methyl-3-quinolin-2-ylmethyl-thiazolidin-4-one;
2-(2,6-Dimethoxy-phenyl)-3-(6-methoxy-naphthalen-2-ylmethyl)-5-methyl-thiazolidin-4-one;
2-(2,6-Dimethoxy-phenyl)-3-(6-methoxy-naphthalen-2-ylmethyl)-thiazolidin-4-one;
2-(4-Fluoro-2,6-dimethoxy-phenyl)-3-(4-trifluoromethoxy-benzyl)-thiazolidin-4-one;
2-(2,6-Dimethoxy-4-methyl-phenyl)-3-(4-trifluoromethoxy-benzyl)-thiazolidin-4-one;
2-(2,6-Dimethoxy-4-methyl-phenyl)-5-methyl-3-(4-trifluoromethoxy-benzyl)-thiazolidin-4-one;
2-(4-Fluoro-2,6-dimethoxy-phenyl)-5-methyl-3-(4-trifluoromethoxy-benzyl)-thiazolidin-4-one;
2-[2-(2-Hydroxy-ethoxy)-6-methoxy-phenyl]-3-(4-trifluoromethoxy-benzyl)-thiazolidin-4-one;
2-[2-(2-Hydroxy-ethoxy)-6-methoxy-phenyl]-3-(4-trifluoromethoxy-benzyl)-thiazolidin-4-one;
2-[2-(2-Hydroxy-ethoxy)-6-methoxy-phenyl]-3-(4-isopropoxy-benzyl)-thiazolidin-4-on;
2-(2,6-Dimethoxy-phenyl)-3-[4-(pyrimidin-5-yloxy)-benzyl]-thiazolidin-4-one;
2-(2,6-Dimethoxy-phenyl)-3-[4-(pyridin-2-yloxy)-benzyl]-thiazolidin-4-one;
2-(2,6-Dimethoxy-phenyl)-3-[4-(pyridin-3-yloxy)-benzyl]-thiazolidin-4-one;
2-(2,6-Dimethoxy-phenyl)-3-[4-(pyrazin-2-yloxy)-benzyl]-thiazolidin-4-one;
2-(2,6-Dimethoxy-phenyl)-3-[4-(6-methyl-pyridazin-3-yloxy)-benzyl]-thiazolidin-4-one;
2-(2,6-Dimethoxy-phenyl)-3-[4-(5-methyl-pyridin-2-yloxy)-benzyl]-thiazolidin-4-one;
2-(2,6-Dimethoxy-phenyl)-3-[4-(6-methoxy-pyridin-2-yloxy)-benzyl]-thiazolidin-4-one;
2-(2,6-Dimethoxy-phenyl)-3-[4-(6-methyl-pyridin-2-yloxy)-benzyl]-thiazolidin-4-one;
2-(2,6-Dimethoxy-phenyl)-3-[4-(4-methyl-pyridin-2-yloxy)-benzyl]-thiazolidin-4-one;
2-(2,6-Dimethoxy-phenyl)-3-[4-(3-methyl-pyridin-2-yloxy)-benzyl]-thiazolidin-4-one;
2-(2,6-Dimethoxy-phenyl)-3-[4-(5-methoxy-pyridin-2-yloxy)-benzyl]-thiazolidin-4-one;
2-(2,6-Dimethoxy-phenyl)-1,1-dioxo-3-(4-trifluoromethoxy-benzyl)-thiazolidin-4-one;
2-(2,6-Dimethoxy-phenyl)-3-(4-isopropoxy-benzyl)-1,1-dioxo-thiazolidin-4-one; and
2-(2,6-Dimethoxy-phenyl)-3-(4-ethoxy-benzyl)-1,1-dioxo-thiazolidin-4-one.

38) A further embodiment of the invention relates to compounds according to embodiment 1), or pharmaceutically acceptable salts thereof, for the prevention or treatment of diseases related to the orexin system; wherein the compounds are selected from the group consisting of:
3-Benzo[1,3]dioxol-5-ylmethyl-2-(2-ethoxy-phenyl)-[1,3]thiazinan-4-one;
2-(2-Ethoxy-phenyl)-3-(4-methoxy-benzyl)-[1,3]thiazinan-4-one;
3-(2-Ethoxy-benzyl)-2-(2-ethoxy-phenyl)-[1,3]thiazinan-4-one;
3-(2-Difluoromethoxy-benzyl)-2-(2-ethoxy-phenyl)-[1,3]thiazinan-4-one;
2-(2-Ethoxy-phenyl)-3-(3-methyl-benzyl)-[1,3]thiazinan-4-one;
2-(2-Ethoxy-phenyl)-3-(3-fluoro-benzyl)[1,3]thiazinan-4-one;
3-(2,6-Dimethoxy-benzyl)-2-(2-ethoxy-phenyl)-[1,3]thiazinan-4-one;
3-(2,3-Dihydro-benzo[1,4]dioxin-6-ylmethyl)-2-(2-ethoxy-phenyl)-[1,3]thiazinan-4-one;
3-(3-Chloro-benzyl)-2-(2-ethoxy-phenyl)-[1,3]thiazinan-4-one;
2-(2-Ethoxy-phenyl)-3-(3-methoxy-benzyl)-[1,3]thiazinan-4-one;
2-(2-Ethoxy-phenyl)-3-(2-fluoro-benzyl)-[1,3]thiazinan-4-one;
3-(4-Chloro-benzyl)-2-(2-ethoxy-phenyl)-[1,3]thiazinan-4-one;
3-(2,5-Difluoro-benzyl)-2-(2-ethoxy-phenyl)-[1,3]thiazinan-4-one;
2-(2-Ethoxy-phenyl)-3-(3-trifluoromethoxy-benzyl)-[1,3]thiazinan-4-one;
3-(2,5-Dimethoxy-benzyl)-2-(2-ethoxy-phenyl)-[1,3]thiazinan-4-one;
3-(3-Chloro-4-fluoro-benzyl)-2-(2-ethoxy-phenyl)-[1,3]thiazinan-4-one;
3-(2-Chloro-benzyl)-2-(2-ethoxy-phenyl)-[1,3]thiazinan-4-one;
2-(2-Ethoxy-phenyl)-3-(4-trifluoromethoxy-benzyl)-[1,3]thiazinan-4-one;
2-(2-Ethoxy-phenyl)-3-(2-methoxy-benzyl)-[1,3]thiazinan-4-one;
2-(2-Ethoxy-phenyl)-3-(2-methyl-benzyl)-[1,3]thiazinan-4-one;
3-(2,3-Dimethoxy-benzyl)-2-(2-ethoxy-phenyl)-[1,3]thiazinan-4-one;
2-(2-Methoxy-phenyl)-3-thiophen-2-ylmethyl-thiazolidin-4-one;
2-(2-Ethoxy-phenyl)-3-thiophen-2-ylmethyl-thiazolidin-4-one;
2-(2-Ethoxy-phenyl)-3-(2-fluoro-benzyl)-thiazolidin-4-one;
2-(2-Ethoxy-phenyl)-3-(4-methoxy-benzyl)-thiazolidin-4-one;
3-(2,5-Difluoro-benzyl)-2-(2-ethoxy-phenyl)-thiazolidin-4-one;
2-(2-Ethoxy-phenyl)-3-(3-fluoro-benzyl)-thiazolidin-4-one;
3-(2,6-Difluoro-benzyl)-2-(2-ethoxy-phenyl)-thiazolidin-4-one;
3-(3,4-Difluoro-benzyl)-2-(2-ethoxy-phenyl)-thiazolidin-4-one;
2-(2-Ethoxy-phenyl)-3-naphthalen-1-ylmethyl-thiazolidin-4-one;
3-(3,5-Difluoro-benzyl)-2-(2-ethoxy-phenyl)-thiazolidin-4-one;
3-(2,5-Difluoro-benzyl)-2-(2,6-dimethoxy-phenyl)-thiazolidin-4-one;
3-(3,5-Difluoro-benzyl)-2-(2,6-dimethoxy-phenyl)-thiazolidin-4-one;
3-Benzo[1,3]dioxol-5-ylmethyl-2-(2-ethoxy-phenyl)-thiazolidin-4-one;
3-(2,3-Dihydro-benzofuran-5-ylmethyl)-2-(2-ethoxy-phenyl)-thiazolidin-4-one;
3-(2,3-Dihydro-benzo[1,4]dioxin-6-ylmethyl)-2-(2-ethoxy-phenyl)-thiazolidin-4-one;
3-(4-Chloro-benzyl)-2-(2-ethoxy-phenyl)-thiazolidin-4-one;
2-(2-Ethoxy-phenyl)-3-(2-trifluoromethyl-benzyl)-thiazolidin-4-one;
2-(2-Ethoxy-phenyl)-3-(2-methoxy-benzyl)-thiazolidin-4-one;
2-(2-Ethoxy-phenyl)-3-(4-methyl-benzyl)-thiazolidin-4-one;
3-(2-Chloro-benzyl)-2-(2-ethoxy-phenyl)-thiazolidin-4-one;
2-(2-Ethoxy-phenyl)-3-(2-methyl-benzyl)-thiazolidin-4-one;
3-(2-Ethoxy-benzyl)-2-(2-ethoxy-phenyl)-thiazolidin-4-one;
3-(3-Chloro-benzyl)-2-(2-ethoxy-phenyl)-thiazolidin-4-one;
2-(2-Ethoxy-phenyl)-3-(3-trifluoromethoxy-benzyl)-thiazolidin-4-one;
2-(2-Ethoxy-phenyl)-3-(4-trifluoromethoxy-benzyl)-thiazolidin-4-one;
3-(2,3-Dimethoxy-benzyl)-2-(2-ethoxy-phenyl)-thiazolidin-4-one;
3-(2,4-Dichloro-benzyl)-2-(2-ethoxy-phenyl)-thiazolidin-4-one;
3-(3,4-Dichloro-benzyl)-2-(2-ethoxy-phenyl)-thiazolidin-4-one;
2-(2-Ethoxy-phenyl)-3-(3-methoxy-benzyl)-thiazolidin-4-one;
3-(2,5-Dimethoxy-benzyl)-2-(2-ethoxy-phenyl)-thiazolidin-4-one;
3-(2,4-Dimethoxy-benzyl)-2-(2,6-dimethoxy-phenyl)-thiazolidin-4-one;
2-(2-Ethoxy-phenyl)-3-(2-trifluoromethoxy-benzyl)-thiazolidin-4-one;
3-(2-Difluoromethoxy-benzyl)-2-(2-ethoxy-phenyl)-thiazolidin-4-one;
3-(2,6-Dimethoxy-benzyl)-2-(2-ethoxy-phenyl)-thiazolidin-4-one;
3-(3,4-Dimethoxy-benzyl)-2-(2-ethoxy-phenyl)-thiazolidin-4-one;
3-(3-Chloro-4-fluoro-benzyl)-2-(2-ethoxy-phenyl)-thiazolidin-4-one;
3-(2-Chloro-4-fluoro-benzyl)-2-(2-ethoxy-phenyl)-thiazolidin-4-one;
2-(2-Ethoxy-phenyl)-3-(3-fluoro-4-methyl-benzyl)-thiazolidin-4-one;
3-(5-Chloro-2-fluoro-benzyl)-2-(2-ethoxy-phenyl)-thiazolidin-4-one;
3-(4-Chloro-2-fluoro-benzyl)-2-(2-ethoxy-phenyl)-thiazolidin-4-one;
3-(4-tert-Butyl-benzyl)-2-(2-ethoxy-phenyl)-thiazolidin-4-one;
2-(2-Ethoxy-phenyl)-3-(2,4,6-trimethyl-benzyl)-thiazolidin-4-one;
3-Benzo[1,3]dioxol-5-ylmethyl-2-(2,6-dimethoxy-phenyl)-thiazolidin-4-one;
2-(2,6-Dimethoxy-phenyl)-3-(2-ethoxy-benzyl)-thiazolidin-4-one;
3-(2,4-Dichloro-benzyl)-2-(2,6-dimethoxy-phenyl)-thiazolidin-4-one;
3-(2-Difluoromethoxy-benzyl)-2-(2,6-dimethoxy-phenyl)-thiazolidin-4-one;
2-(2,6-Dimethoxy-phenyl)-3-(2,4,6-trimethyl-benzyl)-thiazolidin-4-one;
3-(2,3-Dihydro-benzofuran-5-ylmethyl)-2-(2,6-dimethoxy-phenyl)-thiazolidin-4-one;
2-(2,6-Dimethoxy-phenyl)-3-(3-methyl-benzyl)-thiazolidin-4-one;
2-(2,6-Dimethoxy-phenyl)-3-(3-fluoro-benzyl)-thiazolidin-4-one;
3-(3,4-Dichloro-benzyl)-2-(2,6-dimethoxy-phenyl)-thiazolidin-4-one;
3-(2,6-Dimethoxy-benzyl)-2-(2,6-dimethoxy-phenyl)-thiazolidin-4-one;
3-(3,4-Difluoro-benzyl)-2-(2,6-dimethoxy-phenyl)-thiazolidin-4-one;
3-(2,3-Dihydro-benzo[1,4]dioxin-6-ylmethyl)-2-(2,6-dimethoxy-phenyl)-thiazolidin-4-one;
2-(2,6-Dimethoxy-phenyl)-3-(4-methyl-benzyl)-thiazolidin-4-one;
3-(3-Chloro-benzyl)-2-(2,6-dimethoxy-phenyl)-thiazolidin-4-one;
2-(2,6-Dimethoxy-phenyl)-3-(3-methoxy-benzyl)-thiazolidin-4-one;
3-(3,4-Dimethoxy-benzyl)-2-(2,6-dimethoxy-phenyl)-thiazolidin-4-one;
2-(2,6-Dimethoxy-phenyl)-3-(3-fluoro-4-methyl-benzyl)-thiazolidin-4-one;
2-(2,6-Dimethoxy-phenyl)-3-(2-fluoro-benzyl)-thiazolidin-4-one;
3-(4-Chloro-benzyl)-2-(2,6-dimethoxy-phenyl)-thiazolidin-4-one;
2-(2,6-Dimethoxy-phenyl)-3-(3-trifluoromethoxy-benzyl)-thiazolidin-4-one;
3-(2,5-Dimethoxy-benzyl)-2-(2,6-dimethoxy-phenyl)-thiazolidin-4-one;
3-(3-Chloro-4-fluoro-benzyl)-2-(2,6-dimethoxy-phenyl)-thiazolidin-4-one;
3-(5-Chloro-2-fluoro-benzyl)-2-(2,6-dimethoxy-phenyl)-thiazolidin-4-one;
2-(2,6-Dimethoxy-phenyl)-3-(2-trifluoromethyl-benzyl)-thiazolidin-4-one;
3-(2-Chloro-benzyl)-2-(2,6-dimethoxy-phenyl)-thiazolidin-4-one;
3-(2,4-Dimethoxy-benzyl)-2-(2,6-dimethoxy-phenyl)-thiazolidin-4-one;
3-(2,6-Difluoro-benzyl)-2-(2,6-dimethoxy-phenyl)-thiazolidin-4-one;
3-(4-Chloro-2-fluoro-benzyl)-2-(2,6-dimethoxy-phenyl)-thiazolidin-4-one;
2-(2,6-Dimethoxy-phenyl)-3-(2-methoxy-benzyl)-thiazolidin-4-one;
2-(2,6-Dimethoxy-phenyl)-3-(2-methyl-benzyl)-thiazolidin-4-one;
2-(2,6-Dimethoxy-phenyl)-3-(2-trifluoromethoxy-benzyl)-thiazolidin-4-one;
3-(2-Chloro-4-fluoro-benzyl)-2-(2,6-dimethoxy-phenyl)-thiazolidin-4-one;
3-(2,2-Difluoro-benzo[1,3]dioxol-5-ylmethyl)-2-(2,6-dimethoxy-phenyl)-thiazolidin-4-one;
2-(2,6-Dimethoxy-phenyl)-3-(4-trifluoromethyl-benzyl)-thiazolidin-4-one;
2-(2,6-Dimethoxy-phenyl)-3-(4-pyrrol-1-yl-benzyl)-thiazolidin-4-one;
2-(2,6-Dimethoxy-phenyl)-3-(3-fluoro-4-trifluoromethyl-benzyl)-thiazolidin-4-one;
2-(2,6-Dimethoxy-phenyl)-3-(4-pyrrolidin-1-yl-benzyl)-thiazolidin-4-one;
2-(2,6-Dimethoxy-phenyl)-3-(4-pyrazol-1-yl-benzyl)-thiazolidin-4-one;
2-(2,6-Dimethoxy-phenyl)-3-(3,4,5-trifluoro-benzyl)-thiazolidin-4-one;
2-(2,6-Dimethoxy-phenyl)-3-(4-trifluoromethylsulfanyl-benzyl)-thiazolidin-4-one;
3-Benzyl-2-(2-ethoxy-phenyl)-thiazolidin-4-one;
2-(2-Fluoro-6-methoxy-phenyl)-3-naphthalen-2-ylmethyl-thiazolidin-4-one;
2-(2-Ethoxy-phenyl)-3-pyridin-2-ylmethyl-thiazolidin-4-one;
2-(2-Ethoxy-phenyl)-3-naphthalen-2-ylmethyl-thiazolidin-4-one;
2-(2-Ethoxy-phenyl)-3-pyridin-4-ylmethyl-thiazolidin-4-one;
2-(2-Ethoxy-phenyl)-3-(1-methyl-1H-pyrrol-2-ylmethyl)-thiazolidin-4-one;
2-(2,6-Difluoro-phenyl)-3-(4-trifluoromethoxy-benzyl)-thiazolidin-4-one;
2-(2,6-Difluoro-phenyl)-3-naphthalen-2-ylmethyl-thiazolidin-4-one;
2-(2-Methoxy-phenyl)-3-(4-trifluoromethoxy-benzyl)-thiazolidin-4-one;
2-(2-Methoxy-phenyl)-3-naphthalen-2-ylmethyl-thiazolidin-4-one;
2-(2,6-Dimethoxy-phenyl)-3-naphthalen-1-ylmethyl-thiazolidin-4-one;
3-Benzyl-2-(2,6-dimethoxy-phenyl)-thiazolidin-4-one;
2-(2-Fluoro-6-methoxy-phenyl)-3-naphthalen-1-ylmethyl-thiazolidin-4-one;
2-(2-Fluoro-6-methoxy-phenyl)-3-thiophen-2-ylmethyl-thiazolidin-4-one;
2-(2-Fluoro-6-methoxy-phenyl)-3-(4-trifluoromethoxy-benzyl)-thiazolidin-4-one;
3-Benzyl-2-(2-fluoro-6-methoxy-phenyl)thiazolidin-4-one;
2-(2-Fluoro-6-methoxy-phenyl)-3-(1-methyl-1H-pyrrol-2-ylmethyl)-thiazolidin-4-one;
3-Benzyl-5-methyl-2-phenyl-thiazolidin-4-one;
3-(4-Ethoxy-benzyl)-5-methyl-2-phenyl-thiazolidin-4-one;
5-Methyl-3-naphthalen-2-ylmethyl-2-phenyl-thiazolidin-4-one;
3-Benzyl-2-(2-methoxy-phenyl)-5-methyl-thiazolidin-4-one;
3-(4-Ethoxy-benzyl)-2-(2-methoxy-phenyl)-5-methyl-thiazolidin-4-one;
2-(2-Methoxy-phenyl)-5-methyl-3-naphthalen-2-ylmethyl-thiazolidin-4-one;
3-Benzyl-2-(2-ethoxy-phenyl)-5-methyl-thiazolidin-4-one;
3-(4-Ethoxy-benzyl)-2-(2-ethoxy-phenyl)-5-methyl-thiazolidin-4-one;
2-(2-Ethoxy-phenyl)-5-methyl-3-naphthalen-2-ylmethyl-thiazolidin-4-one;
3-Benzyl-2-(2,6-dimethoxy-phenyl)-5-methyl-thiazolidin-4-one;
2-(2,6-Dimethoxy-phenyl)-5-methyl-3-(4-trifluoromethyl-benzyl)-thiazolidin-4-one;
3-Benzyl-2-(2-methoxy-phenyl)-5,5-dimethyl-thiazolidin-4-one;
3-(4-Ethoxy-benzyl)-2-(2-methoxy-phenyl)-5,5-dimethyl-thiazolidin-4-one;
2-(2-Methoxy-phenyl)-5,5-dimethyl-3-naphthalen-2-ylmethyl-thiazolidin-4-one;
3-Benzyl-2-(2-ethoxy-phenyl)-5,5-dimethyl-thiazolidin-4-one;
2-(2-Ethoxy-phenyl)-5,5-dimethyl-3-naphthalen-2-ylmethyl-thiazolidin-4-one;
3-Benzyl-2-(2,6-dimethoxy-phenyl)-5,5-dimethyl-thiazolidin-4-one;
2-(2,6-Dimethoxy-phenyl)-5,5-dimethyl-3-(4-trifluoromethyl-benzyl)-thiazolidin-4-one;
2-(2,6-Dimethoxy-phenyl)-3-(4-ethoxy-benzyl)-5,5-dimethyl-thiazolidin-4-one;
2-(2,6-Dimethoxy-phenyl)-5,5-dimethyl-3-naphthalen-2-ylmethyl-thiazolidin-4-one;
3-(4-Ethoxy-benzyl)-2-(2-methoxy-6-methyl-phenyl)-thiazolidin-4-one;
2-(2,6-Diethoxy-phenyl)-3-(4-ethoxy-benzyl)-thiazolidin-4-one;
2-(2-Chloro-6-methoxy-phenyl)-3-(4-ethoxy-benzyl)-thiazolidin-4-one;
2-(2-Methoxy-6-methyl-phenyl)-3-(4-trifluoromethoxy-benzyl)-thiazolidin-4-one;
2-(2,6-Diethoxy-phenyl)-3-(4-trifluoromethoxy-benzyl)-thiazolidin-4-one;
2-(2-Chloro-6-methoxy-phenyl)-3-(4-trifluoromethoxy-benzyl)-thiazolidin-4-one;
3-(4-Benzyloxy-benzyl)-2-(2,6-dimethoxy-phenyl)-thiazolidin-4-one;
2-(2,6-Dimethoxy-phenyl)-3-[4-(2-hydroxy-ethoxy)-benzyl]-thiazolidin-4-one;
2-(2,6-Dimethoxy-phenyl)-3-[4-(2-methoxy-ethoxy)-benzyl]-thiazolidin-4-one;
2-(2-Methoxy-phenyl)-5-methyl-3-(4-trifluoromethoxy-benzyl)thiazolidin-4-one;
2-(2-Ethoxy-phenyl)-5-methyl-3-(4-trifluoromethoxy-benzyl)-thiazolidin-4-one;
2-(2,6-Dimethoxy-phenyl)-5,5-dimethyl-3-(4-trifluoromethoxy-benzyl)-thiazolidin-4-one;
3-Benzyl-2-(5-bromo-benzo[1,3]dioxol-4-yl)-thiazolidin-4-one;
3-Benzyl-2-(2-methoxy-naphthalen-1-yl)-thiazolidin-4-one;
3-Benzyl-2-(2,2-dimethyl-2,3-dihydro-benzofuran-7-yl)-thiazolidin-4-one;
3-Benzyl-2-(2,3-dihydro-benzo[1,4]dioxin-5-yl)-thiazolidin-4-one;
2-Benzo[1,3]dioxol-4-yl-3-benzyl-thiazolidin-4-one;
3-Benzyl-2-(2-methoxy-5-methyl-phenyl)thiazolidin-4-one;
3-Benzyl-2-(5-benzyl-2-methoxy-phenyl)thiazolidin-4-one;
2-(2,5-Dimethoxy-phenyl)-3-(4-trifluoromethoxy-benzyl)-thiazolidin-4-one;
2-(5-Bromo-benzo[1,3]dioxol-4-yl)-3-(4-trifluoromethoxy-benzyl)-thiazolidin-4-one;
2-(2-Methoxy-naphthalen-1-yl)-3-(4-trifluoromethoxy-benzyl)-thiazolidin-4-one;
2-(4-Hydroxy-2-methoxy-phenyl)-3-(4-trifluoromethoxy-benzyl)-thiazolidin-4-one;
2-Benzo[1,3]dioxol-4-yl-3-(4-trifluoromethoxy-benzyl)-thiazolidin-4-one;
2-(2-Methoxy-5-methyl-phenyl)-3-(4-trifluoromethoxy-benzyl)-thiazolidin-4-one;
2-(6-Methoxy-indan-5-yl)-3-(4-trifluoromethoxy-benzyl)-thiazolidin-4-one;
3-Biphenyl-4-ylmethyl-2-(2,6-dimethoxy-phenyl)-thiazolidin-4-one;
2-(6-Methoxy-quinolin-5-yl)-3-(4-trifluoromethoxy-benzyl)-thiazolidin-4-one;
2-(2,6-Dimethoxy-phenyl)-3-(6-ethoxy-pyridin-3-ylmethyl)-thiazolidin-4-one;
2-(2,6-Dimethoxy-phenyl)-3-(5-ethoxy-pyridin-2-ylmethyl)-thiazolidin-4-one;
2-(2,6-Dimethoxy-phenyl)-3-(6-ethoxy-pyridazin-3-ylmethyl)-thiazolidin-4-one;
2-(2,3-Dihydro-benzo[1,4]dioxin-5-yl)-3-(4-trifluoromethoxy-benzyl)-thiazolidin-4-one;
3-Benzyl-2-(1H-indol-2-yl)-thiazolidin-4-one;
2-(2-Ethoxy-pyridin-3-yl)-3-(4-trifluoromethoxy-benzyl)-thiazolidin-4-one;
2-(1-Methyl-1H-indazol-3-yl)-3-(4-trifluoromethoxy-benzyl)-thiazolidin-4-one;
2-(6-Methoxy-pyridin-2-yl)-3-(4-trifluoromethoxy-benzyl)-thiazolidin-4-one;
3-(4-Ethoxy-benzyl)-2-(2-ethoxy-pyridin-3-yl)-thiazolidin-4-one;
2-(2-Methoxy-pyridin-3-yl)-3-(4-trifluoromethoxy-benzyl)-thiazolidin-4-on;
2-(4,6-Dimethoxy-pyrimidin-5-yl)-3-(4-ethoxy-benzyl)-thiazolidin-4-one;
3-Benzyl-2-(6-methoxy-pyridin-2-yl)-thiazolidin-4-one;
2-(4,6-Dimethoxy-pyrimidin-5-yl)-3-(4-trifluoromethoxy-benzyl)-thiazolidin-4-one;
3-Naphthalen-2-ylmethyl-2,3-dihydro-[2,2′]bithiazolyl-4-one;
3-Benzyl-2-(2-methoxy-phenyl)-thiazolidin-4-one;
3-Benzyl-2-(1-methyl-1H-indol-3-yl)-thiazolidin-4-one;
2-(2,6-Dimethoxy-phenyl)-3-thiophen-2-ylmethyl-thiazolidin-4-one;
3-Thiophen-2-ylmethyl-2-(2-trifluoromethyl-phenyl)-thiazolidin-4-one;
2-(2,6-Dimethoxy-phenyl)-5-methyl-3-quinoxalin-2-ylmethyl-thiazolidin-4-one;
2-(2,6-Dimethoxy-phenyl)-3-quinoxalin-2-ylmethyl-thiazolidin-4-one;
3-Benzo[b]thiophen-6-ylmethyl-2-(2,6-dimethoxy-phenyl)-5-methyl-thiazolidin-4-one;
2-(2,6-Dimethoxy-phenyl)-5-methyl-3-(1-methyl-1H-indol-2-ylmethyl)-thiazolidin-4-one;
2-(2,6-Dimethoxy-phenyl)-5-methyl-3-(1-methyl-1H-indol-6-ylmethyl)-thiazolidin-4-one;
3-Benzo[b]thiophen-5-ylmethyl-2-(2,6-dimethoxy-phenyl)-thiazolidin-4-one;
2-(2,6-Dimethoxy-phenyl)-5-methyl-3-(1-methyl-1H-benzoimidazol-2-ylmethyl)-thiazolidin-4-one;
2-(2,6-Dimethoxy-phenyl)-5-methyl-3-quinolin-6-ylmethyl-thiazolidin-4-one;
3-Benzo[b]thiophen-2-ylmethyl-2-(2,6-dimethoxy-phenyl)-5-methyl-thiazolidin-4-one;
3-Benzo[b]thiophen-3-ylmethyl-2-(2,6-dimethoxy-phenyl)-5-methyl-thiazolidin-4-one;
2-(2,6-Dimethoxy-phenyl)-3-(1H-indol-2-ylmethyl)-5-methyl-thiazolidin-4-one;
2-(2,6-Dimethoxy-phenyl)-5-methyl-3-quinolin-7-ylmethyl-thiazolidin-4-one;
3-Benzothiazol-2-ylmethyl-2-(2,6-dimethoxy-phenyl)-5-methyl-thiazolidin-4-one;
2-(2,6-Dimethoxy-phenyl)-5-methyl-3-quinoxalin-6-ylmethyl-thiazolidin-4-one;
3-Benzofuran-2-ylmethyl-2-(2,6-dimethoxy-phenyl)-5-methyl-thiazolidin-4-one;
2-(2,6-Dimethoxy-phenyl)-3-(1H-indol-3-ylmethyl)-5-methyl-thiazolidin-4-one;
2-(2,6-Dimethoxy-phenyl)-3-quinoxalin-6-ylmethyl-thiazolidin-4-one;
2-(3-Fluoro-2,6-dimethoxy-phenyl)-5-methyl-3-(4-trifluoromethoxy-benzyl)-thiazolidin-4-one;
2-(2,6-Dimethoxy-phenyl)-3-(1-methyl-1H-indol-2-ylmethyl)-thiazolidin-4-one;
3-Benzo[b]thiophen-2-ylmethyl-2-(2,6-dimethoxy-phenyl)-thiazolidin-4-one;
2-(2,6-Dimethoxy-phenyl)-3-(1-methyl-1H-indol-6-ylmethyl)-thiazolidin-4-one;
3-(4-Trifluoromethoxy-benzyl)-2-(2,4,6-trimethoxy-phenyl)-thiazolidin-4-one;
2-(3-Fluoro-2,6-dimethoxy-phenyl)-3-(4-trifluoromethoxy-benzyl)-thiazolidin-4-one;
5-Methyl-3-(4-trifluoromethoxy-benzyl)-2-(2,3,6-trimethoxy-phenyl)-thiazolidin-4-one;
2-(2,6-Dimethoxy-3-methyl-phenyl)-5-methyl-3-(4-trifluoromethoxy-benzyl)-thiazolidin-4-one;
3-Benzo[b]thiophen-3-ylmethyl-2-(2,6-dimethoxy-phenyl)-thiazolidin-4-one;
5-Methyl-3-(4-trifluoromethoxy-benzyl)-2-(2,4,6-trimethoxy-phenyl)-thiazolidin-4-one;
2-(2,6-Dimethoxy-3-methyl-phenyl)-3-(4-trifluoromethoxy-benzyl)-thiazolidin-4-one;
2-(2,6-Dimethoxy-phenyl)-3-quinolin-7-ylmethyl-thiazolidin-4-one;
2-(2,6-Dimethoxy-phenyl)-3-(1H-indol-2-ylmethyl)-thiazolidin-4-one;
2-(2,6-Dimethoxy-phenyl)-3-(1-methyl-1H-benzoimidazol-2-ylmethyl)-thiazolidin-4-one;
3-(1H-Benzoimidazol-2-ylmethyl)-2-(4-benzyloxy-3-methoxy-phenyl)-thiazolidin-4-one;
2-(2,6-Dimethoxy-phenyl)-3-quinolin-6-ylmethyl-thiazolidin-4-one;
3-Benzothiazol-2-ylmethyl-2-(2,6-dimethoxy-phenyl)-thiazolidin-4-one;
2-(3-Chloro-2,6-dimethoxy-phenyl)-5-methyl-3-(4-trifluoromethoxy-benzyl)-thiazolidin-4-one;
3-(4-Trifluoromethoxy-benzyl)-2-(2,3,6-trimethoxy-phenyl)-thiazolidin-4-one;
3-Benzofuran-2-ylmethyl-2-(2,6-dimethoxy-phenyl)-thiazolidin-4-one;
2-(2,6-Dimethoxy-phenyl)-5-methyl-3-(1-methyl-1H-benzotriazol-5-ylmethyl)-thiazolidin-4-one;
3-(1H-Benzoimidazol-2-ylmethyl)-2-(2,6-dimethoxy-phenyl)-thiazolidin-4-one;
2-(3-Chloro-2,6-dimethoxy-phenyl)-3-(4-trifluoromethoxy-benzyl)-thiazolidin-4-one;
2-(2,6-Dimethoxy-phenyl)-3-(1H-indol-3-ylmethyl)-thiazolidin-4-one;
2-(2-Fluoro-4,6-dimethoxy-phenyl)-3-(4-trifluoromethoxy-benzyl)-thiazolidin-4-one;
3-(1H-Benzoimidazol-2-ylmethyl)-2-(4-benzyloxy-3-methoxy-phenyl)-5-methyl-thiazolidin-4-one;
2-(2-Chloro-4,6-dimethoxy-phenyl)-3-(4-trifluoromethoxy-benzyl)-thiazolidin-4-one;
2-[2-(S)-(2,3-Dihydroxy-propoxy)-6-methoxy-phenyl]-3-(4-trifluoromethoxy-benzyl)-thiazolidin-4-one;
2-[2-(R)-(2,3-Dihydroxy-propoxy)-6-methoxy-phenyl]-3-(4-trifluoromethoxy-benzyl)-thiazolidin-4-one;
2-[2-Methoxy-6-(2-methoxy-ethoxy)-phenyl]-3-(4-trifluoromethoxy-benzyl)-thiazolidin-4-one;
2-[2-(2-Hydroxy-propoxy)-6-methoxy-phenyl]-3-(4-trifluoromethoxy-benzyl)-thiazolidin-4-one;
3-(4-Ethoxy-benzyl)-2-[2-(2-hydroxy-propoxy)-6-methoxy-phenyl]-thiazolidin-4-one;
2-[2-(2-Hydroxy-propoxy)-6-methoxy-phenyl]-3-(4-isopropoxy-benzyl)-thiazolidin-4-one;
2-(2,6-Dimethoxy-phenyl)-3-[4-(6-trifluoromethyl-pyridin-2-yloxy)-benzyl]-thiazolidin-4-one; and
2-(2,6-Dimethoxy-phenyl)-1-oxo-3-(4-trifluoromethoxy-benzyl)-thiazolidin-4-one.

The compounds of formulae (I) and (II) and their pharmaceutically acceptable salts can be used as medicaments, e.g. in the form of pharmaceutical compositions for enteral or parental administration.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention or treatment of a disease or disorder mentioned herein comprising administering to a subject a pharmaceutically active amount of a compound of formulae (I) or (II).

For avoidance of any doubt, if compounds are described as useful for the prevention or treatment of certain diseases, such compounds are likewise suitable for use in the preparation of a medicament for the prevention or treatment of said diseases.

The compounds according to formulae (I) and (II) are useful for the prevention or treatment of diseases related to the orexin system.

Such diseases related to the orexin system may be selected from the group consisting of all types of sleep disorders, of stress-related syndromes, of addictions (especially psychoactive substance use, abuse, seeking and reinstatement), of cognitive dysfunctions in the healthy population and in psychiatric and neurologic disorders, of eating or drinking disorders.

In a sub-embodiment, such diseases related to the orexin system may be selected from the group consisting of sleep disorders that comprises all types of insomnias, narcolepsy and other disorders of excessive sleepiness, sleep-related dystonias, restless leg syndrome, sleep apneas, jet-lag syndrome, shift-work syndrome, delayed or advanced sleep phase syndrome or insomnias related to psychiatric disorders (notably all types of insomnias, especially primary insomnia).

In another sub-embodiment, such diseases related to the orexin system may be selected from the group consisting of cognitive dysfunctions that comprise deficits in all types of attention, learning and memory functions occurring transiently or chronically in the normal, healthy, young, adult or aging population, and also occurring transiently or chronically in psychiatric, neurologic, cardiovascular and immune disorders.

In another sub-embodiment, such diseases related to the orexin system may be selected from the group consisting of eating disorders that comprise metabolic dysfunction; dysregulated appetite control; compulsive obesities; emeto-bulimia or anorexia nervosa.

In another sub-embodiment, such diseases related to the orexin system may be selected from the group consisting of all types of addictions (especially psychoactive substance use, abuse, seeking and reinstatement) that comprise all types of psychological or physical addictions and their related tolerance and dependence components.

Eating disorders may be defined as comprising metabolic dysfunction; dysregulated appetite control; compulsive obesities; emeto-bulimia or anorexia nervosa. Pathologically modified food intake may result from disturbed appetite (attraction or aversion for food); altered energy balance (intake vs. expenditure); disturbed perception of food quality (high fat or carbohydrates, high palatability); disturbed food availability (unrestricted diet or deprivation) or disrupted water balance. Drinking disorders include polydipsias in psychiatric disorders and all other types of excessive fluid intake.

Sleep disorders include all types of parasomnias, insomnias, narcolepsy and other disorders of excessive sleepiness, sleep-related dystonias; restless leg syndrome; sleep apneas; jet-lag syndrome; shift-work syndrome, delayed or advanced sleep phase syndrome or insomnias related to psychiatric disorders.

Insomnias are defined as comprising sleep disorders associated with aging; intermittent treatment of chronic insomnia; situational transient insomnia (new environment, noise) or short-term insomnia due to stress; grief; pain or illness. Insomnia also include stress-related syndromes including post-traumatic stress disorders as well as other types and subtypes of anxiety disorders such as generalized anxiety, obsessive compulsive disorder, panic attacks and all types of phobic anxiety and avoidance.

Addictions may be defined as addiction to one or more rewarding stimuli, notably to one rewarding stimulus. Such rewarding stimuli may be of either natural or synthetic origin. Psychoactive substance use, abuse, seeking and reinstatement are defined as all types of psychological or physical addictions and their related tolerance and dependence components.

Cognitive dysfunctions include deficits in all types of attention, learning and memory functions occurring transiently or chronically in the normal, healthy, young, adult or aging population, and also occurring transiently or chronically in psychiatric, neurologic, cardiovascular and immune disorders.

Besides, any characteristics described in this invention for the compounds of formula (I) (whether for the compounds themselves, salts thereof, compositions containing the compounds or salts thereof, uses of the compounds or salts thereof, etc.) apply mutatis mutandis to compounds of formula (I_{E1}), formula (I_{E2}), and formula (II).

### Preparation of Compounds of Formula (I):

A further aspect of the invention is a process for the preparation of compounds of formula (I). Compounds according to formula (I) of the present invention can be prepared according to the general sequence of reactions outlined in the schemes below wherein X, Y, R¹, R², R³, and R⁴ are as defined for formula (I). Other abbreviations used herein are explicitly defined, or are as defined in the experimental section. In some instances the generic groups X, Y, R¹, R², R³, and R⁴ might be incompatible with the assembly illustrated in the schemes below and so will require the use of protecting groups (PG). The use of protecting groups is well known in the art (see for example “Protective Groups in Organic Synthesis”, T. W. Greene, P. G. M. Wuts, Wiley-Interscience, 1999). For the purposes of this discussion, it will be assumed that such protecting groups as necessary are in place. The compounds obtained may also be converted into pharmaceutically acceptable salts thereof in a manner known per se.

In general, all chemical transformations can be performed according to well-known standard methodologies as described in the literature or as described in the procedures below.

Thiazolidin-4-one or thiazinan-4-one derivatives (X represents S) of formula (I) may be prepared according to scheme 1

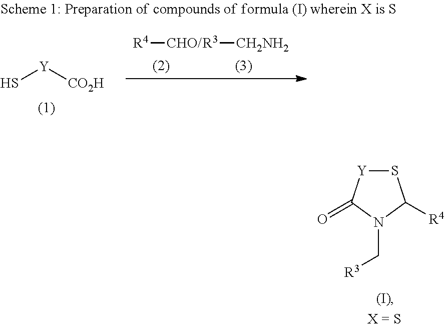

These compounds of formula (I) can be synthetised in a one-pot three-component reaction involving an mercapto-acid (1), an aldehyde (2) and an amine (3) in the presence of a base such as DIPEA, a coupling reagent such as HBTU in an aprotic solvent such as DMF (Saraf S. K. et al European Journal of Medicinal Chemistry 2008, 43, 897-905; Rawal R. K. et al Journal of Chemical Research 2004, 5, 368-369).

1-Oxo and 1,1-dioxo-thiazolidin-4-one derivatives (X represents S(O) or SO₂) may be prepared by oxidation of the corresponding thiazolidin-4-one derivative with an appropriate amount of an oxidant such as mCPBA in an aprotic solvent such as DCM as depicted in scheme 2.

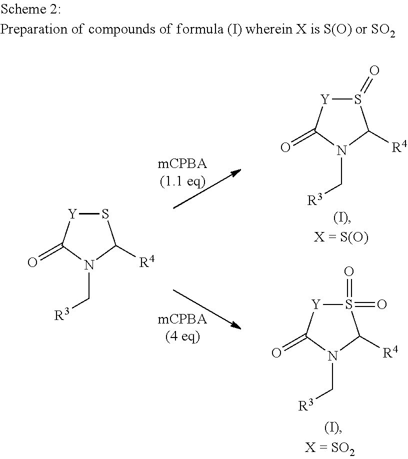

Amines of formula R³—CH₂—NH₂ and aldehydes of formula R⁴—CHO are commercially available, well known in the art, or readily available from commercially available precursors. Procedures to transform precursor functional groups into such required amines or aldehydes, such as reduction of carboxylic acids, esters, amides, nitriles; oxidation of alcohols; substitution of halides or equivalent activated alcohols (eg. via methane-/toluene-sulphonates); reductive amination of aldehydes; or sequential metallation/formylation of aromatic halides are well known in the art (literature for precursors of heteroaryl-containing groups: see e.g. T. Eicher, S. Hauptmann “The chemistry of Heterocycles: Structure, Reactions, Syntheses, and Applications”, 2nd Edition 2003, Wiley, ISBN 978-3-527-30720-3; A. R. Katrizky, C. W. Rees, E. F. V. Scriven (Eds.) “Comprehensive Heterocyclic Chemistry II” 1996, Elsevier, ISBN 0-08-042072-9).

In some instances, substituents may also be introduced in a final step onto an appropriate (eg. phenolic) precursor molecule. The hydroxy group of such phenol precursor may be alkylated using standard procedures, or arylated using standard procedures such as the Ullmann reaction with halide derivatives of formula Ar³-L¹ in the presence of CuCl, 2,2,6,6-tetramethyl-heptane-3,5-dione and a base such as Cs₂CO₃ in an aprotic solvent such as NMP (WO2006/0173049).

The synthesis of some particular aldehydes of formula R⁴—CHO is described in the following schemes 3 to 6.

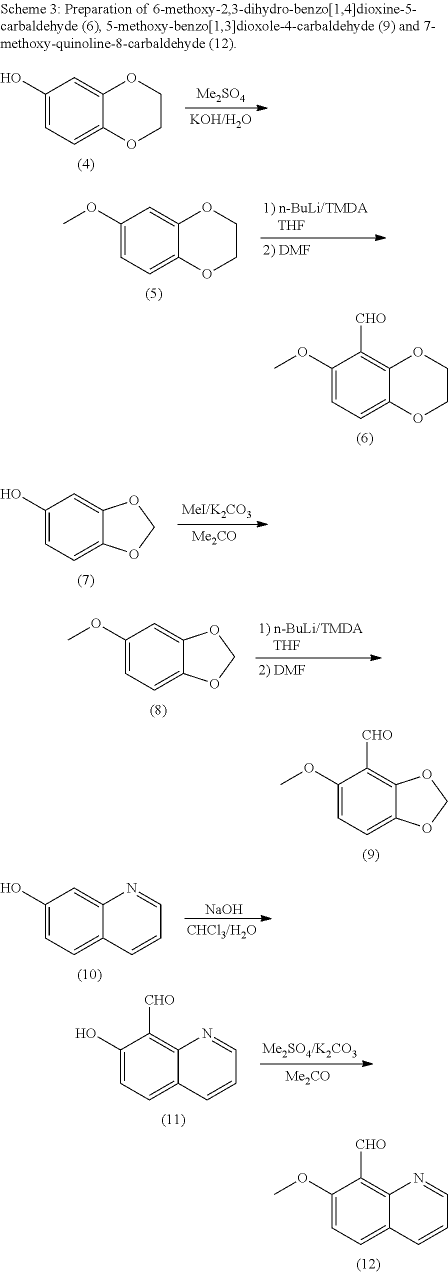

Methylation of commercially available 2,3-dihydro-benzo[1,4]dioxin-6-ol (4) with dimethyl sulfate gives 6-methoxy-2,3-dihydro-benzo[1,4]dioxine (5) (Guillaumet G. et al. Eur. J. Med. Chem. 1990, 25, 1, 45-51). Formylation with n-BuLi/DMF in the presence of TMDA in an aprotic solvent such as THF affords the aldehyde (6) (Guillaumet G. et al. J. Heterocyclic. Chem. 1989, 26, 1, 193-197).

Methylation of commercially benzo[1,3]dioxol-5-ol (7) with methyl iodide in the presence of a base such as K₂CO₃ in an aprotic solvent such as acetone affords 5-methoxy-benzo[1,3]dioxole (8) (Schuda P. F. et al, J. Org. Chem. 1987, 52, 10, 1972-1979). Formylation with n-BuLi/DMF in the presence of TMDA in an aprotic solvent such as THF affords aldehyde (9) (Guillaumet G. et al. J. Heterocyclic. Chem. 1989, 26, 1, 193-197).

Riemer-Tiemann reaction with CHCl₃ in aq. NaOH of the commercially available 7-hydroxyquinoline (10) gives the aldehyde (11). Methylation with dimethyl sulfate affords 7-methoxy-quinoline-8-carbaldehyde (12) (U.S. Pat. No. 4,342,771).

Benzooxazole and benzo[d]isoxazole aldehyde derivatives of formula R⁴—CHO may for instance be synthesised according to scheme 4.

Reaction of commercially available 3-amino-2,6-dihydroxy-benzoic acid methyl ester (13) with triethyl orthoacetate in the presence of PTSA gives the ester (14) (WO2006/069155). Methylation with dimethyl sulfate in the presence of a base such as K₂CO₃ in an aprotic solvent such as acetone affords compound (15). Reduction with LAH in an aprotic solvent such as THF gives the alcohol (16) which can be oxidized with MnO₂ in DCM to give 6-methoxy-2-methyl-benzooxazole-7-carbaldehyde (17).

Duff formylation of 3-methyl-benzo[d]isoxazol-6-ol (18) with urotropin in AcOH (Elkasaby M. A. et al Indian Journal of Chemistry 1980, 19B(7), 571-575) gives the aldehyde (19) (Kumari S. S. et al Indian Journal of Chemistry 1986, 25B(8), 870-871). Methylation with dimethyl sulfate affords 6-methoxy-3-methyl-benzo[d]isoxazole-7-carbaldehyde (20).

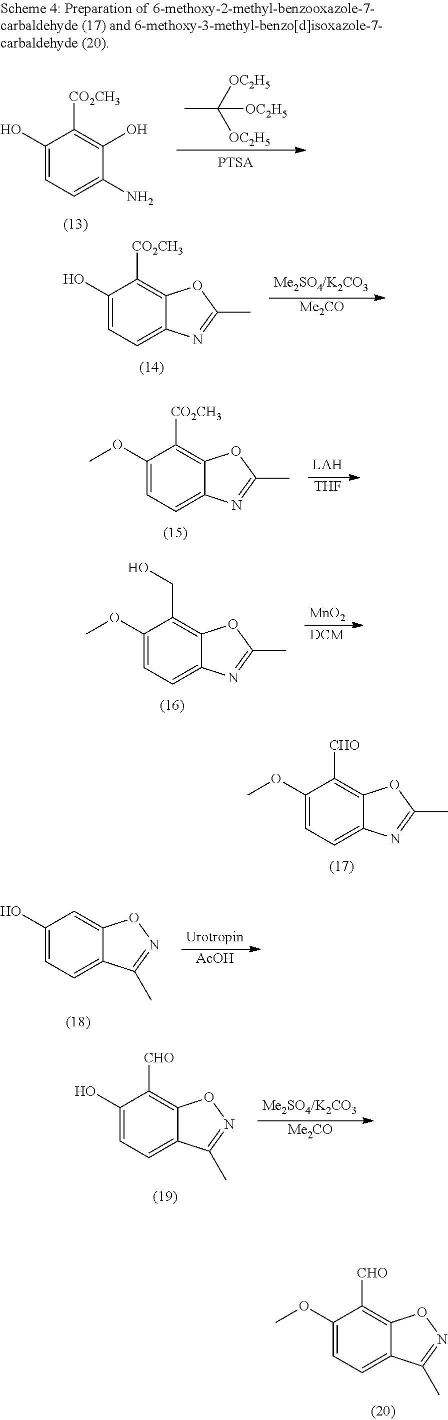

Further synthetic methods for the preparation of aldehydes R⁴—CHO are described below for the specific examples shown in schemes 5 and 6.

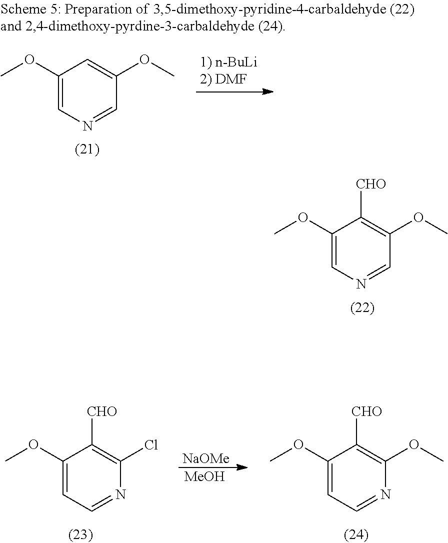

Formylation with n-BuLi/DMF in an aprotic solvent such as THF of the commercially available 3,5-dimethoxypyridine (21) affords 3,5-dimethoxy-pyridine-4-carbaldehyde (22) (U.S. Pat. No. 6,555,557).

Reaction of the commercially available 2-chloro-4-methoxy-pyridine-3-carbaldehyde (23) with NaOMe in MeOH affords 2,4-dimethoxy-pyridine-3-carbaldehyde (24).

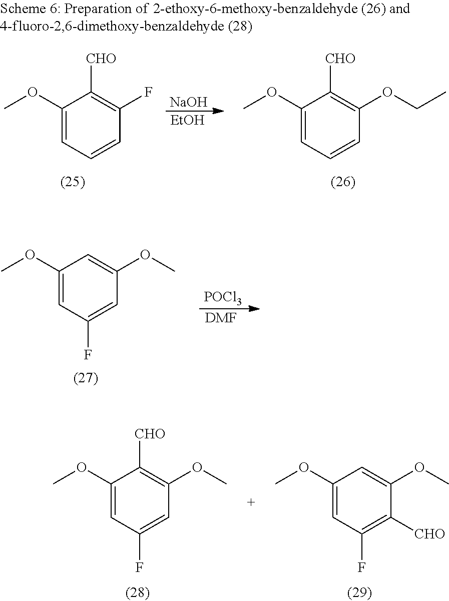

Reaction of commercially 2-fluoro-6-methoxy-benzaldehyde (25) with NaOH in EtOH affords the aldehyde (26) (U.S. Pat. No. 4,367,234).

Vilsmeier-Haack reaction with POCl₃ in dry DMF of commercially available 1-fluoro-3,5-dimethoxy-benzene (27) gives a mixture of aldehyde (28) and aldehyde (29) with a ratio of about 1/9 (Stanjeck V. et al. Helvetica Chimica Acta 1998, 81, 9, 1596-1607).

Whenever the compounds of formula (I) or (II) are obtained in the form of mixtures of enantiomers, the enantiomers can be separated using methods known to the one skilled in the art: e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in presence or absence of an amine such as triethylamine, diethylamine) and eluent B (hexane), at a flow rate of 0.8 to 150 mL/min.

### EXPERIMENTAL SECTION

Abbrevations (as used herein and in the description before):

Ac acetyl (as in Ac₂O=acetic acid anhydride; AcOH=acetic acid)
aq. aqueous
BSA bovine serum albumine
CHO Chinese hamster ovary
conc. concentrated
d day(s)
DCM dichloromethane
DIPEA diisopropylethylamine

### DMF N,N-dimethylformamide

eq equivalent(s)
ES electron spray
ether diethylether
EtOAc ethyl acetate
EtOH ethanol
FC flash chromatography on silica gel
FCS foatal calf serum
FLIPR fluorescent imaging plate reader
h hour(s)
HBTU O-(benzotriazol-1-yl)-1,1,3,3-tetramethyl-uronium hexafluorphoshate
HBSS Hank's balanced salt solution
HEPES 4-(2-hydroxyethyl)-piperazine-1-ethanesulfonic acid
HPLC high performance liquid chromatography
LAH lithium aluminium hydride
LC liquid chromatography
M molar(ity)
mCPBA 3-chloroperoxybenzoic acid
Me methyl
MeCN acetonitrile
MeOH methanol
min minute(s)
MS mass spectroscopy
MW microwave
n-BuLi n-butyllithium
NMP N-methyl-2-pyrrolidinone
PTSA (para-) p-toluenesulfonic acid
prep. preparative
RT room temperature
sat. saturated
t_{R} retention time
TFA trifluoroacetic acid
THF tetrahydrofuran
TMDA N,N,N′,N′-tetramethylethylenediamine

### I—Chemistry

All temperatures are stated in ° C. Compounds are characterized by LC-MS (Finnigan Navigator with HP 1100 Binary Pump and DAD, column: 4.6×50 mm, Zorbax SB-AQ, 5 μm, 120 Å, using two conditions: basic: eluent A: MeCN, eluent B: conc. NH₃ in water (1.0 mL/L), 5% to 95% CH₃CN; acidic: eluent A: MeCN, eluent B: TFA in water (0.4 mL/L), 5% to 95% CH₃CN), t_{R} is given in min; by TLC (TLC-plates from Merck, Silica gel 60 F₂₅₄); or by melting point. Compounds are purified by column chromatography on silica gel or by preparative HPLC (column: X-terra RP18, 50×19 mm, 5 μm, gradient: 10-95% MeCN in water containing 0.5% of formic acid).

The following examples illustrate the preparation of compounds of the invention but do not at all limit the scope thereof. All compounds were obtained in racemic form, or, in case two or more chiral centers are present, as mixture of stereoisomers.

### A.1 Synthesis of thiazolidin-4-one or thiazinan-4-one Derivatives (General Procedure)

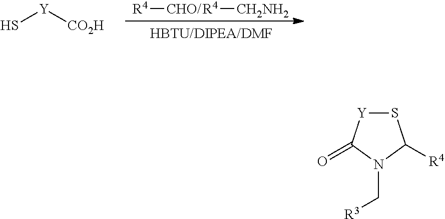

A mixture of the respective aldehyde (0.1 mmol) and the respective amine (0.1 mmol) in dry DMF (2 mL) was stirred at RT for 20 min. followed by the addition of the respective mercapto-acid (0.3 mmol, 3 eq). After 30 min. of stirring at RT, DIPEA (0.2 mmol, 2 eq) and HBTU (0.2 mmol, 2 eq) were added and the stirring at RT was continued overnight. The reaction mixture was then diluted with EtOAc, washed with 5% aq. citric acid, water, sat. NaHCO₃, brine, dried over MgSO₄, filtered and concentrated under reduced pressure to yield a crude solid. The products were purified by prep. HPLC to provide the final compound.

For the thiazolidin-4-one derivatives (X═S, Y═CH₂, CHR¹, CR¹R²), the final compounds were prepared by condensation of the respective aldehyde, and the respective amine with the respective mercapto acetic acid derivative.

For the thiazinan-4-one derivatives (X═S, Y═CH₂CH₂), the final compounds were prepared by condensation of the respective aldehyde, and the respective amine with the respective 3-mercapto propionic acid.

### PREPARATION OF EXAMPLES

The following Examples were synthesized according to the above general procedure:

| Example | Name | [M + H]⁺ | t_{R} |
|---|---|---|---|
| 1 | 2-(2,6-Dimethoxy-phenyl)-3-(4-methoxy-benzyl)- | 360.16 | 0.96 |
| | thiazolidin-4-one | | |
| 2 | 2-(2,6-Dimethoxy-phenyl)-3-(4-trifluoromethoxy- | 414.06 | 0.93 |
| | benzyl)-thiazolidin-4-one | | |
| 3 | 3-(4-tert-Butoxy-benzyl)-2-(2,6-dimethoxy-phenyl)- | 402.05 | 0.97 |
| | thiazolidin-4-one | | |
| 4 | 3-(4-Difluoromethoxy-benzyl)-2-(2,6-dimethoxy- | 395.97 | 0.91 |
| | phenyl)-thiazolidin-4-one | | |
| 5 | 2-(2,6-Dimethoxy-phenyl)-3-(4-isopropoxy-benzyl)- | 388.09 | 0.96 |
| | thiazolidin-4-one | | |
| 6 | 2-(2,6-Dimethoxy-phenyl)-3-(4-ethoxy-benzyl)- | 374.09 | 0.92 |
| | thiazolidin-4-one | | |
| 7 | 2-(2,6-Dimethoxy-phenyl)-3-(4-phenoxy-benzyl)- | 422.1 | 1.00 |
| | thiazolidin-4-one | | |
| 8 | 2-(2,6-Dimethoxy-phenyl)-3-naphthalen-2-ylmethyl- | 380.16 | 0.79 |
| | thiazolidin-4-one | | |
| 9 | 3-(4-Ethoxy-benzyl)-2-(6-methoxy-2,3-dihydro- | 401.98 | 1.01 |
| | benzo[1,4]dioxin-5-yl)-thiazolidin-4-one | | |
| 10 | 2-(6-Methoxy-2,3-dihydro-benzo[1,4]dioxin-5-yl)-3- | 441.86 | 1.05 |
| | (4-trifluoromethoxy-benzyl)-thiazolidin-4-one | | |
| 11 | 3-(4-Ethoxy-benzyl)-2-(5-methoxy- | 387.99 | 1.02 |
| | benzo[1,3]dioxol-4-yl)-thiazolidin-4-one | | |
| 12 | 2-(5-Methoxy-benzo[1,3]dioxol-4-yl)-3-(4- | 427.9 | 1.05 |
| | trifluoromethoxy-benzyl)-thiazolidin-4-one | | |
| 13 | 2-(2,6-Dimethoxy-phenyl)-3-(4-ethoxy-benzyl)-5-methyl- | 388.01 | 0.78 |
| | thiazolidin-4-one | | |
| 14 | 2-(2,6-Dimethoxy-phenyl)-5-methyl-3-naphthalen- | 394 | 0.81 |
| | 2-ylmethyl-thiazolidin-4-one | | |
| 15 | 3-(4-Ethoxy-benzyl)-2-(2-ethoxy-6-methoxy- | 388.01 | 1.05 |
| | phenyl)-thiazolidin-4-one | | |
| 16 | 2-(2-Ethoxy-6-methoxy-phenyl)-3-(4- | 427.74 | 1.09 |
| | trifluoromethoxy-benzyl)-thiazolidin-4-one | | |
| 17 | 2-(2,6-Dimethoxy-phenyl)-5-methyl-3-(4- | 428.01 | 0.82 |
| | trifluoromethoxy-benzyl)-thiazolidin-4-one | | |
| 18 | 2-(2,6-Dimethoxy-phenyl)-3-[4-(2,2,2-trifluoro- | 427.89 | 0.93 |
| | ethoxy)-benzyl]-thiazolidin-4-one | | |
| 19 | 3-(4-Ethoxy-benzyl)-2-(6-methoxy-3-methyl- | 399.02 | 0.89 |
| | benzo[d]isoxazol-7-yl)-thiazolidin-4-one | | |
| 20 | 3-(4-Ethoxy-benzyl)-2-(6-methoxy-2-methyl- | 399.05 | 0.86 |
| | benzooxazol-7-yl)-thiazolidin-4-one | | |
| 21 | 3-(4-Ethoxy-benzyl)-2-(7-methoxy-quinolin-8-yl)- | 394.92 | 0.92 |
| | thiazolidin-4-one | | |
| 22 | 2-(6-Methoxy-3-methyl-benzo[d]isoxazol-7-yl)-3-(4- | 438.99 | 0.94 |
| | trifluoromethoxy-benzyl)-thiazolidin-4-one | | |
| 23 | 2-(6-Methoxy-2-methyl-benzooxazol-7-yl)-3-(4- | 438.91 | 0.91 |
| | trifluoromethoxy-benzyl)-thiazolidin-4-one | | |
| 24 | 2-(7-Methoxy-quinolin-8-yl)-3-(4-trifluoromethoxy- | 434.92 | 0.97 |
| | benzyl)-thiazolidin-4-one | | |
| 25 | 2-(2,4-Dimethoxy-pyridin-3-yl)-3-(4- | 414.93 | 0.91 |
| | trifluoromethoxy-benzyl)-thiazolidin-4-one | | |
| 26 | 2-(2,4-Dimethoxy-pyridin-3-yl)-3-(4-ethoxy-benzyl)- | 375.04 | 0.85 |
| | thiazolidin-4-one | | |
| 27 | 2-(2,6-Dimethoxy-phenyl)-3-quinolin-2-ylmethyl- | 381.15 | 0.89 |
| | thiazolidin-4-one | | |
| 28 | 2-(3,5-Dimethoxy-pyridin-4-yl)-3-(4- | 415.03 | 0.89 |
| | trifluoromethoxy-benzyl)-thiazolidin-4-one | | |
| 29 | 2-(3,5-Dimethoxy-pyridin-4-yl)-3-(4-ethoxy-benzyl)- | 375.14 | 0.84 |
| | thiazolidin-4-one | | |
| 30 | 2-(2,6-Dimethoxy-phenyl)-5-methyl-3-quinolin-2- | 395.05 | 0.92 |
| | ylmethyl-thiazolidin-4-one | | |
| 31 | 2-(2,6-Dimethoxy-phenyl)-3-(6-methoxy- | 424.53 | 1.11 |
| | naphthalen-2-ylmethyl)-5-methyl-thiazolidin-4-one | | |
| 32 | 2-(2,6-Dimethoxy-phenyl)-3-(6-methoxy- | 410.2 | 1.05 |
| | naphthalen-2-ylmethyl)-thiazolidin-4-one | | |
| 33 | 2-(4-Fluoro-2,6-dimethoxy-phenyl)-3-(4- | 432.44 | 1.1 |
| | trifluoromethoxy-benzyl)-thiazolidin-4-one | | |
| 34 | 2-(2,6-Dimethoxy-4-methyl-phenyl)-3-(4- | 428.49 | 1.15 |
| | trifluoromethoxy-benzyl)-thiazolidin-4-one | | |
| 35 | 2-(2,6-Dimethoxy-4-methyl-phenyl)-5-methyl-3-(4- | 442 | 1.21 |
| | trifluoromethoxy-benzyl)-thiazolidin-4-one | | |
| 36 | 2-(4-Fluoro-2,6-dimethoxy-phenyl)-5-methyl-3-(4- | 446.46 | 1.17 |
| | trifluoromethoxy-benzyl)-thiazolidin-4-one | | |

The following further Examples were synthesized according to the above general procedure:

| Example | Name | [M + H]⁺ | t_{R} |
|---|---|---|---|
| 37 | 3-Benzo[1,3]dioxol-5-ylmethyl-2-(2-ethoxy-phenyl)- | 372.12 | 0.9 |
| | [1,3]thiazinan-4-one | | |
| 38 | 2-(2-Ethoxy-phenyl)-3-(4-methoxy-benzyl)- | 358.15 | 0.91 |
| | [1,3]thiazinan-4-one | | |
| 39 | 3-(2-Ethoxy-benzyl)-2-(2-ethoxy-phenyl)- | 372.14 | 0.97 |
| | [1,3]thiazinan-4-one | | |
| 40 | 3-(2-Difluoromethoxy-benzyl)-2-(2-ethoxy-phenyl)- | 393.83 | 0.93 |
| | [1,3]thiazinan-4-one | | |
| 41 | 2-(2-Ethoxy-phenyl)-3-(3-methyl-benzyl)- | 342.2 | 0.96 |
| | [1,3]thiazinan-4-one | | |
| 42 | 2-(2-Ethoxy-phenyl)-3-(3-fluoro-benzyl)- | 346.17 | 0.93 |
| | [1,3]thiazinan-4-one | | |
| 43 | 3-(2,6-Dimethoxy-benzyl)-2-(2-ethoxy-phenyl)- | 388.14 | 0.9 |
| | [1,3]thiazinan-4-one | | |
| 44 | 3-(2,3-Dihydro-benzo[1,4]dioxin-6-ylmethyl)-2-(2-ethoxy- | 386.12 | 0.9 |
| | phenyl)-[1,3]thiazinan-4-one | | |
| 45 | 3-(3-Chloro-benzyl)-2-(2-ethoxy-phenyl)- | 361.87 | 0.97 |
| | [1,3]thiazinan-4-one | | |
| 46 | 2-(2-Ethoxy-phenyl)-3-(3-methoxy-benzyl)- | 358.21 | 0.92 |
| | [1,3]thiazinan-4-one | | |
| 47 | 2-(2-Ethoxy-phenyl)-3-(2-fluoro-benzyl)- | 346.17 | 0.93 |
| | [1,3]thiazinan-4-one | | |
| 48 | 3-(4-Chloro-benzyl)-2-(2-ethoxy-phenyl)- | 361.94 | 0.98 |
| | [1,3]thiazinan-4-one | | |
| 49 | 3-(2,5-Difluoro-benzyl)-2-(2-ethoxy-phenyl)- | 364.12 | 0.94 |
| | [1,3]thiazinan-4-one | | |
| 50 | 2-(2-Ethoxy-phenyl)-3-(3-trifluoromethoxy-benzyl)- | 412.06 | 1 |
| | [1,3]thiazinan-4-one | | |
| 51 | 3-(2,5-Dimethoxy-benzyl)-2-(2-ethoxy-phenyl)- | 388.14 | 0.92 |
| | [1,3]thiazinan-4-one | | |
| 52 | 3-(3-Chloro-4-fluoro-benzyl)-2-(2-ethoxy-phenyl)- | 380.09 | 0.98 |
| | [1,3]thiazinan-4-one | | |
| 53 | 3-(2-Chloro-benzyl)-2-(2-ethoxy-phenyl)- | 361.87 | 0.97 |
| | [1,3]thiazinan-4-one | | |
| 54 | 2-(2-Ethoxy-phenyl)-3-(4-trifluoromethoxy-benzyl)- | 412.05 | 1 |
| | [1,3]thiazinan-4-one | | |
| 55 | 2-(2-Ethoxy-phenyl)-3-(2-methoxy-benzyl)- | 358.17 | 0.93 |
| | [1,3]thiazinan-4-one | | |
| 56 | 2-(2-Ethoxy-phenyl)-3-(2-methyl-benzyl)- | 342.19 | 0.96 |
| | [1,3]thiazinan-4-one | | |
| 57 | 3-(2,3-Dimethoxy-benzyl)-2-(2-ethoxy-phenyl)- | 388.15 | 0.91 |
| | [1,3]thiazinan-4-one | | |
| 58 | 2-(2-Methoxy-phenyl)-3-thiophen-2-ylmethyl- | 306.18 | 0.71 |
| | thiazolidin-4-one | | |
| 59 | 2-(2-Ethoxy-phenyl)-3-thiophen-2-ylmethyl- | 320.18 | 0.82 |
| | thiazolidin-4-one | | |
| 60 | 2-(2-Ethoxy-phenyl)-3-(2-fluoro-benzyl)-thiazolidin- | 332.15 | 0.94 |
| | 4-one | | |
| 61 | 2-(2-Ethoxy-phenyl)-3-(4-methoxy-benzyl)- | 344.16 | 0.93 |
| | thiazolidin-4-one | | |
| 62 | 3-(2,5-Difluoro-benzyl)-2-(2-ethoxy-phenyl)- | 350.1 | 0.95 |
| | thiazolidin-4-one | | |
| 63 | 2-(2-Ethoxy-phenyl)-3-(3-fluoro-benzyl)-thiazolidin- | 332.16 | 0.95 |
| | 4-one | | |
| 64 | 3-(2,6-Difluoro-benzyl)-2-(2-ethoxy-phenyl)- | 350.15 | 0.92 |
| | thiazolidin-4-one | | |
| 65 | 3-(3,4-Difluoro-benzyl)-2-(2-ethoxy-phenyl)- | 350.14 | 0.96 |
| | thiazolidin-4-one | | |
| 66 | 2-(2-Ethoxy-phenyl)-3-naphthalen-1-ylmethyl- | 364.2 | 0.67 |
| | thiazolidin-4-one | | |
| 67 | 3-(3,5-Difluoro-benzyl)-2-(2-ethoxy-phenyl)- | 350.14 | 0.96 |
| | thiazolidin-4-one | | |
| 68 | 3-(2,5-Difluoro-benzyl)-2-(2,6-dimethoxy-phenyl)- | 366.11 | 0.88 |
| | thiazolidin-4-one | | |
| 69 | 3-(3,5-Difluoro-benzyl)-2-(2,6-dimethoxy-phenyl)- | 366.12 | 0.88 |
| | thiazolidin-4-one | | |
| 70 | 3-Benzo[1,3]dioxol-5-ylmethyl-2-(2-ethoxy-phenyl)- | 358.14 | 0.91 |
| | thiazolidin-4-one | | |
| 71 | 3-(2,3-Dihydro-benzofuran-5-ylmethyl)-2-(2-ethoxy- | 356.17 | 0.93 |
| | phenyl)-thiazolidin-4-one | | |
| 72 | 3-(2,3-Dihydro-benzo[1,4]dioxin-6-ylmethyl)-2-(2- | 372.13 | 0.92 |
| | ethoxy-phenyl)-thiazolidin-4-one | | |
| 73 | 3-(4-Chloro-benzyl)-2-(2-ethoxy-phenyl)-thiazolidin- | 348.13 | 0.99 |
| | 4-one | | |
| 74 | 2-(2-Ethoxy-phenyl)-3-(2-trifluoromethyl-benzyl)- | 382.07 | 0.99 |
| | thiazolidin-4-one | | |
| 75 | 2-(2-Ethoxy-phenyl)-3-(2-methoxy-benzyl)- | 344.17 | 0.94 |
| | thiazolidin-4-one | | |
| 76 | 2-(2-Ethoxy-phenyl)-3-(4-methyl-benzyl)- | 328.23 | 0.98 |
| | thiazolidin-4-one | | |
| 77 | 3-(2-Chloro-benzyl)-2-(2-ethoxy-phenyl)-thiazolidin- | 348.13 | 0.98 |
| | 4-one | | |
| 78 | 2-(2-Ethoxy-phenyl)-3-(2-methyl-benzyl)- | 328.17 | 0.98 |
| | thiazolidin-4-one | | |
| 79 | 3-(2-Ethoxy-benzyl)-2-(2-ethoxy-phenyl)- | 358.19 | 0.98 |
| | thiazolidin-4-one | | |
| 80 | 3-(3-Chloro-benzyl)-2-(2-ethoxy-phenyl)-thiazolidin- | 348.08 | 0.99 |
| | 4-one | | |
| 81 | 2-(2-Ethoxy-phenyl)-3-(3-trifluoromethoxy-benzyl)- | 398.01 | 1 |
| | thiazolidin-4-one | | |
| 82 | 2-(2-Ethoxy-phenyl)-3-(4-trifluoromethoxy-benzyl)- | 398.01 | 1.01 |
| | thiazolidin-4-one | | |
| 83 | 3-(2,3-Dimethoxy-benzyl)-2-(2-ethoxy-phenyl)- | 374.15 | 0.93 |
| | thiazolidin-4-one | | |
| 84 | 3-(2,4-Dichloro-benzyl)-2-(2-ethoxy-phenyl)- | 382.01 | 1.04 |
| | thiazolidin-4-one | | |
| 85 | 3-(3,4-Dichloro-benzyl)-2-(2-ethoxy-phenyl)- | 382.02 | 1.03 |
| | thiazolidin-4-one | | |
| 86 | 2-(2-Ethoxy-phenyl)-3-(3-methoxy-benzyl)- | 344.22 | 0.93 |
| | thiazolidin-4-one | | |
| 87 | 3-(2,5-Dimethoxy-benzyl)-2-(2-ethoxy-phenyl)- | 374.15 | 0.93 |
| | thiazolidin-4-one | | |
| 88 | 3-(2,4-Dimethoxy-benzyl)-2-(2,6-dimethoxy- | 374.15 | 0.93 |
| | phenyl)-thiazolidin-4-one | | |
| 89 | 2-(2-Ethoxy-phenyl)-3-(2-trifluoromethoxy-benzyl)- | 398.02 | 1 |
| | thiazolidin-4-one | | |
| 90 | 3-(2-Difluoromethoxy-benzyl)-2-(2-ethoxy-phenyl)- | 380.08 | 0.94 |
| | thiazolidin-4-one | | |
| 91 | 3-(2,6-Dimethoxy-benzyl)-2-(2-ethoxy-phenyl)- | 374.16 | 0.92 |
| | thiazolidin-4-one | | |
| 92 | 3-(3,4-Dimethoxy-benzyl)-2-(2-ethoxy-phenyl)- | 374.16 | 0.91 |
| | thiazolidin-4-one | | |
| 93 | 3-(3-Chloro-4-fluoro-benzyl)-2-(2-ethoxy-phenyl)- | 366.02 | 0.99 |
| | thiazolidin-4-one | | |
| 94 | 3-(2-Chloro-4-fluoro-benzyl)-2-(2-ethoxy-phenyl)- | 366.04 | 0.99 |
| | thiazolidin-4-one | | |
| 95 | 2-(2-Ethoxy-phenyl)-3-(3-fluoro-4-methyl-benzyl)- | 346.18 | 0.99 |
| | thiazolidin-4-one | | |
| 96 | 3-(5-Chloro-2-fluoro-benzyl)-2-(2-ethoxy-phenyl)- | 366.08 | 0.98 |
| | thiazolidin-4-one | | |
| 97 | 3-(4-Chloro-2-fluoro-benzyl)-2-(2-ethoxy-phenyl)- | 366.05 | 1 |
| | thiazolidin-4-one | | |
| 98 | 3-(4-tert-Butyl-benzyl)-2-(2-ethoxy-phenyl)- | 370.19 | 1.08 |
| | thiazolidin-4-one | | |
| 99 | 2-(2-Ethoxy-phenyl)-3-(2,4,6-trimethyl-benzyl)- | 356.19 | 1.04 |
| | thiazolidin-4-one | | |
| 100 | 3-Benzo[1,3]dioxol-5-ylmethyl-2-(2,6-dimethoxy- | 374.14 | 0.83 |
| | phenyl)-thiazolidin-4-one | | |
| 101 | 2-(2,6-Dimethoxy-phenyl)-3-(2-ethoxy-benzyl)- | 374.14 | 0.93 |
| | thiazolidin-4-one | | |
| 102 | 3-(2,4-Dichloro-benzyl)-2-(2,6-dimethoxy-phenyl)- | 398.01 | 0.99 |
| | thiazolidin-4-one | | |
| 103 | 3-(2-Difluoromethoxy-benzyl)-2-(2,6-dimethoxy- | 396.01 | 0.99 |
| | phenyl)-thiazolidin-4-one | | |
| 104 | 2-(2,6-Dimethoxy-phenyl)-3-(2,4,6-trimethyl- | 372.12 | 0.87 |
| | benzyl)-thiazolidin-4-one | | |
| 105 | 3-(2,3-Dihydro-benzofuran-5-ylmethyl)-2-(2,6- | 372.14 | 0.95 |
| | dimethoxy-phenyl)-thiazolidin-4-one | | |
| 106 | 2-(2,6-Dimethoxy-phenyl)-3-(3-methyl-benzyl)- | 344.11 | 0.98 |
| | thiazolidin-4-one | | |
| 107 | 2-(2,6-Dimethoxy-phenyl)-3-(3-fluoro-benzyl)- | 346.18 | 0.93 |
| | thiazolidin-4-one | | |
| 108 | 3-(3,4-Dichloro-benzyl)-2-(2,6-dimethoxy-phenyl)- | 398.11 | 0.9 |
| | thiazolidin-4-one | | |
| 109 | 3-(2,6-Dimethoxy-benzyl)-2-(2,6-dimethoxy- | 390.15 | 0.93 |
| | phenyl)-thiazolidin-4-one | | |
| 110 | 3-(3,4-Difluoro-benzyl)-2-(2,6-dimethoxy-phenyl)- | 366.09 | 0.97 |
| | thiazolidin-4-one | | |
| 111 | 3-(2,3-Dihydro-benzo[1,4]dioxin-6-ylmethyl)-2-(2,6- | 388.05 | 0.88 |
| | dimethoxy-phenyl)-thiazolidin-4-one | | |
| 112 | 2-(2,6-Dimethoxy-phenyl)-3-(4-methyl-benzyl)- | 344.08 | 0.99 |
| | thiazolidin-4-one | | |
| 113 | 3-(3-Chloro-benzyl)-2-(2,6-dimethoxy-phenyl)- | 364.18 | 0.94 |
| | thiazolidin-4-one | | |
| 114 | 2-(2,6-Dimethoxy-phenyl)-3-(3-methoxy-benzyl)- | 360.06 | 0.95 |
| | thiazolidin-4-one | | |
| 115 | 3-(3,4-Dimethoxy-benzyl)-2-(2,6-dimethoxy- | 390.08 | 0.95 |
| | phenyl)-thiazolidin-4-one | | |
| 116 | 2-(2,6-Dimethoxy-phenyl)-3-(3-fluoro-4-methyl- | 362.04 | 0.95 |
| | benzyl)-thiazolidin-4-one | | |
| 117 | 2-(2,6-Dimethoxy-phenyl)-3-(2-fluoro-benzyl)- | 348.09 | 0.93 |
| | thiazolidin-4-one | | |
| 118 | 3-(4-Chloro-benzyl)-2-(2,6-dimethoxy-phenyl)- | 364.04 | 0.94 |
| | thiazolidin-4-one | | |
| 119 | 2-(2,6-Dimethoxy-phenyl)-3-(3-trifluoromethoxy- | 414.08 | 0.91 |
| | benzyl)-thiazolidin-4-one | | |
| 120 | 3-(2,5-Dimethoxy-benzyl)-2-(2,6-dimethoxy- | 390.16 | 0.89 |
| | phenyl)-thiazolidin-4-one | | |
| 121 | 3-(3-Chloro-4-fluoro-benzyl)-2-(2,6-dimethoxy- | 382.02 | 0.99 |
| | phenyl)-thiazolidin-4-one | | |
| 122 | 3-(5-Chloro-2-fluoro-benzyl)-2-(2,6-dimethoxy- | 382.12 | 0.98 |
| | phenyl)-thiazolidin-4-one | | |
| 123 | 2-(2,6-Dimethoxy-phenyl)-3-(2-trifluoromethyl- | 398.12 | 0.97 |
| | benzyl)-thiazolidin-4-one | | |
| 124 | 3-(2-Chloro-benzyl)-2-(2,6-dimethoxy-phenyl)- | 364.06 | 0.92 |
| | thiazolidin-4-one | | |
| 125 | 3-(2,4-Dimethoxy-benzyl)-2-(2,6-dimethoxy- | 390.14 | 0.99 |
| | phenyl)-thiazolidin-4-one | | |
| 126 | 3-(2,6-Difluoro-benzyl)-2-(2,6-dimethoxy-phenyl)- | 366.08 | 0.96 |
| | thiazolidin-4-one | | |
| 127 | 3-(4-Chloro-2-fluoro-benzyl)-2-(2,6-dimethoxy- | 382.08 | 0.97 |
| | phenyl)-thiazolidin-4-one | | |
| 128 | 2-(2,6-Dimethoxy-phenyl)-3-(2-methoxy-benzyl)- | 360.04 | 0.9 |
| | thiazolidin-4-one | | |
| 129 | 2-(2,6-Dimethoxy-phenyl)-3-(2-methyl-benzyl)- | 344.14 | 0.89 |
| | thiazolidin-4-one | | |
| 130 | 2-(2,6-Dimethoxy-phenyl)-3-(2-trifluoromethoxy- | 440.07 | 0.94 |
| | benzyl)-thiazolidin-4-one | | |
| 131 | 3-(2-Chloro-4-fluoro-benzyl)-2-(2,6-dimethoxy- | 382.09 | 0.97 |
| | phenyl)-thiazolidin-4-one | | |
| 132 | 3-(2,2-Difluoro-benzo[1,3]dioxol-5-ylmethyl)-2-(2,6- | 409.95 | 0.96 |
| | dimethoxy-phenyl)-thiazolidin-4-one | | |
| 133 | 2-(2,6-Dimethoxy-phenyl)-3-(4-trifluoromethyl- | 397.84 | 0.96 |
| | benzyl)-thiazolidin-4-one | | |
| 134 | 2-(2,6-Dimethoxy-phenyl)-3-(4-pyrrol-1-yl-benzyl)- | 394.63 | 0.95 |
| | thiazolidin-4-one | | |
| 135 | 2-(2,6-Dimethoxy-phenyl)-3-(3-fluoro-4- | 415.77 | 0.97 |
| | trifluoromethyl-benzyl)-thiazolidin-4-one | | |
| 136 | 2-(2,6-Dimethoxy-phenyl)-3-(4-pyrrolidin-1-yl- | 399.07 | 1 |
| | benzyl)-thiazolidin-4-one | | |
| 137 | 2-(2,6-Dimethoxy-phenyl)-3-(4-pyrazol-1-yl- | 396 | 0.86 |
| | benzyl)-thiazolidin-4-one | | |
| 138 | 2-(2,6-Dimethoxy-phenyl)-3-(3,4,5-trifluoro-benzyl)- | 384 | 0.94 |
| | thiazolidin-4-one | | |
| 139 | 2-(2,6-Dimethoxy-phenyl)-3-(4- | 429.95 | 1.01 |
| | trifluoromethylsulfanyl-benzyl)thiazolidin-4-one | | |
| 140 | 3-Benzyl-2-(2-ethoxy-phenyl)-thiazolidin-4-one | 314.26 | 0.76 |
| 141 | 2-(2-Fluoro-6-methoxy-phenyl)-3-naphthalen-2- | 368.13 | 0.79 |
| | ylmethyl-thiazolidin-4-one | | |
| 142 | 2-(2-Ethoxy-phenyl)-3-pyridin-2-ylmethyl- | 315.26 | 0.74 |
| | thiazolidin-4-one | | |
| 143 | 2-(2-Ethoxy-phenyl)-3-naphthalen-2-ylmethyl- | 364.18 | 0.64 |
| | thiazolidin-4-one | | |
| 144 | 2-(2-Ethoxy-phenyl)-3-pyridin-4-ylmethyl- | 315.22 | 0.73 |
| | thiazolidin-4-one | | |
| 145 | 2-(2-Ethoxy-phenyl)-3-(1-methyl-1H-pyrrol-2- | 317.23 | 0.73 |
| | ylmethyl)-thiazolidin-4-one | | |
| 146 | 2-(2,6-Difluoro-phenyl)-3-(4-trifluoromethoxy- | 390.11 | 0.81 |
| | benzyl)-thiazolidin-4-one | | |
| 147 | 2-(2,6-Difluoro-phenyl)-3-naphthalen-2-ylmethyl- | 355.71 | 0.79 |
| | thiazolidin-4-one | | |
| 148 | 2-(2-Methoxy-phenyl)-3-(4-trifluoromethoxy- | 384.07 | 0.8 |
| | benzyl)-thiazolidin-4-one | | |
| 149 | 2-(2-Methoxy-phenyl)-3-naphthalen-2-ylmethyl- | 350.11 | 0.79 |
| | thiazolidin-4-one | | |
| 150 | 2-(2,6-Dimethoxy-phenyl)-3-naphthalen-1-ylmethyl- | 380.18 | 0.79 |
| | thiazolidin-4-one | | |
| 151 | 3-Benzyl-2-(2,6-dimethoxy-phenyl)-thiazolidin-4- | 330.1 | 0.72 |
| | one | | |
| 152 | 2-(2-Fluoro-6-methoxy-phenyl)-3-naphthalen-1- | 368.14 | 0.79 |
| | ylmethyl-thiazolidin-4-one | | |
| 153 | 2-(2-Fluoro-6-methoxy-phenyl)-3-thiophen-2- | 324.14 | 0.71 |
| | ylmethyl-thiazolidin-4-one | | |
| 154 | 2-(2-Fluoro-6-methoxy-phenyl)-3-(4- | 401.92 | 1.07 |
| | trifluoromethoxy-benzyl)-thiazolidin-4-one | | |
| 155 | 3-Benzyl-2-(2-fluoro-6-methoxy-phenyl)-thiazolidin- | 318.13 | 0.72 |
| | 4-one | | |
| 156 | 2-(2-Fluoro-6-methoxy-phenyl)-3-(1-methyl-1H- | 321.18 | 0.7 |
| | pyrrol-2-ylmethyl)-thiazolidin-4-one | | |
| 157 | 3-Benzyl-5-methyl-2-phenyl-thiazolidin-4-one | 284.13 | 0.74 |
| 158 | 3-(4-Ethoxy-benzyl)-5-methyl-2-phenyl-thiazolidin- | 328.09 | 0.78 |
| | 4-one | | |
| 159 | 5-Methyl-3-naphthalen-2-ylmethyl-2-phenyl- | 334.14 | 0.81 |
| | thiazolidin-4-one | | |
| 160 | 3-Benzyl-2-(2-methoxy-phenyl)-5-methyl- | 314.1 | 0.75 |
| | thiazolidin-4-one | | |
| 161 | 3-(4-Ethoxy-benzyl)-2-(2-methoxy-phenyl)-5- | 358.13 | 0.78 |
| | methyl-thiazolidin-4-one | | |
| 162 | 2-(2-Methoxy-phenyl)-5-methyl-3-naphthalen-2- | 364 | 0.81 |
| | ylmethyl-thiazolidin-4-one | | |
| 163 | 3-Benzyl-2-(2-ethoxy-phenyl)-5-methyl-thiazolidin- | 328.09 | 0.78 |
| | 4-one | | |
| 164 | 3-(4-Ethoxy-benzyl)-2-(2-ethoxy-phenyl)-5-methyl- | 372.07 | 0.81 |
| | thiazolidin-4-one | | |
| 165 | 2-(2-Ethoxy-phenyl)-5-methyl-3-naphthalen-2- | 378.12 | 0.84 |
| | ylmethyl-thiazolidin-4-one | | |
| 166 | 3-Benzyl-2-(2,6-dimethoxy-phenyl)-5-methyl- | 344.03 | 0.75 |
| | thiazolidin-4-one | | |
| 167 | 2-(2,6-Dimethoxy-phenyl)-5-methyl-3-(4- | 411.97 | 0.81 |
| | trifluoromethyl-benzyl)-thiazolidin-4-one | | |
| 168 | 3-Benzyl-2-(2-methoxy-phenyl)-5,5-dimethyl- | 328.09 | 0.78 |
| | thiazolidin-4-one | | |
| 169 | 3-(4-Ethoxy-benzyl)-2-(2-methoxy-phenyl)-5,5- | 372.05 | 0.81 |
| | dimethyl-thiazolidin-4-one | | |
| 170 | 2-(2-Methoxy-phenyl)-5,5-dimethyl-3-naphthalen-2- | 378.07 | 0.84 |
| | ylmethyl-thiazolidin-4-one | | |
| 171 | 3-Benzyl-2-(2-ethoxy-phenyl)-5,5-dimethyl- | 342.08 | 0.81 |
| | thiazolidin-4-one | | |
| 172 | 2-(2-Ethoxy-phenyl)-5,5-dimethyl-3-naphthalen-2- | 392.07 | 0.86 |
| | ylmethyl-thiazolidin-4-one | | |
| 173 | 3-Benzyl-2-(2,6-dimethoxy-phenyl)-5,5-dimethyl- | 330.1 | 0.72 |
| | thiazolidin-4-one | | |
| 174 | 2-(2,6-Dimethoxy-phenyl)-5,5-dimethyl-3-(4- | 426.02 | 0.83 |
| | trifluoromethyl-benzyl)-thiazolidin-4-one | | |
| 175 | 2-(2,6-Dimethoxy-phenyl)-3-(4-ethoxy-benzyl)-5,5- | 401.65 | 0.8 |
| | dimethyl-thiazolidin-4-one | | |
| 176 | 2-(2,6-Dimethoxy-phenyl)-5,5-dimethyl-3- | 408.02 | 0.83 |
| | naphthalen-2-ylmethyl-thiazolidin-4-one | | |
| 177 | 3-(4-Ethoxy-benzyl)-2-(2-methoxy-6-methyl- | 358.01 | 1.04 |
| | phenyl)-thiazolidin-4-one | | |
| 178 | 2-(2,6-Diethoxy-phenyl)-3-(4-ethoxy-benzyl)- | 402.05 | 1.07 |
| | thiazolidin-4-one | | |
| 179 | 2-(2-Chloro-6-methoxy-phenyl)-3-(4-ethoxy- | 377.91 | 1.04 |
| | benzyl)-thiazolidin-4-one | | |
| 180 | 2-(2-Methoxy-6-methyl-phenyl)-3-(4- | 438.97 | 1.08 |
| | trifluoromethoxy-benzyl)-thiazolidin-4-one | | |
| 181 | 2-(2,6-Diethoxy-phenyl)-3-(4-trifluoromethoxy- | 442.71 | 1.11 |
| | benzyl)-thiazolidin-4-one | | |
| 182 | 2-(2-Chloro-6-methoxy-phenyl)-3-(4- | 417.81 | 1.08 |
| | trifluoromethoxy-benzyl)-thiazolidin-4-one | | |
| 183 | 3-(4-Benzyloxy-benzyl)-2-(2,6-dimethoxy-phenyl)- | 436.01 | 1.06 |
| | thiazolidin-4-one | | |
| 184 | 2-(2,6-Dimethoxy-phenyl)-3-[4-(2-hydroxy-ethoxy)- | 390.03 | 0.88 |
| | benzyl]-thiazolidin-4-one | | |
| 185 | 2-(2,6-Dimethoxy-phenyl)-3-[4-(2-methoxy-ethoxy)- | 404.04 | 0.98 |
| | benzyl]-thiazolidin-4-one | | |
| 186 | 2-(2-Methoxy-phenyl)-5-methyl-3-(4- | 398.01 | 0.82 |
| | trifluoromethoxy-benzyl)-thiazolidin-4-one | | |
| 187 | 2-(2-Ethoxy-phenyl)-5-methyl-3-(4- | 412.07 | 0.85 |
| | trifluoromethoxy-benzyl)-thiazolidin-4-one | | |
| 188 | 2-(2,6-Dimethoxy-phenyl)-5,5-dimethyl-3-(4- | 442.04 | 0.84 |
| | trifluoromethoxy-benzyl)-thiazolidin-4-one | | |
| 189 | 3-Benzyl-2-(5-bromo-benzo[1,3]dioxol-4-yl)- | 393.9 | 0.75 |
| | thiazolidin-4-one | | |
| 190 | 3-Benzyl-2-(2-methoxy-naphthalen-1-yl)- | 350.11 | 0.77 |
| | thiazolidin-4-one | | |
| 191 | 3-Benzyl-2-(2,2-dimethyl-2,3-dihydro-benzofuran- | 339.78 | 0.78 |
| | 7-yl)-thiazolidin-4-one | | |
| 192 | 3-Benzyl-2-(2,3-dihydro-benzo[1,4]dioxin-5-yl)- | 328.64 | 0.71 |
| | thiazolidin-4-one | | |
| 193 | 2-Benzo[1,3]dioxol-4-yl-3-benzyl-thiazolidin-4-one | 314.12 | 0.7 |
| 194 | 3-Benzyl-2-(2-methoxy-5-methyl-phenyl)- | 314.13 | 0.75 |
| | thiazolidin-4-one | | |
| 195 | 3-Benzyl-2-(5-benzyl-2-methoxy-phenyl)- | 390.05 | 0.83 |
| | thiazolidin-4-one | | |
| 196 | 2-(2,5-Dimethoxy-phenyl)-3-(4-trifluoromethoxy- | 414.02 | 0.8 |
| | benzyl)-thiazolidin-4-one | | |
| 197 | 2-(5-Bromo-benzo[1,3]dioxol-4-yl)-3-(4- | 476.12 | 0.82 |
| | trifluoromethoxy-benzyl)-thiazolidin-4-one | | |
| 198 | 2-(2-Methoxy-naphthalen-1-yl)-3-(4- | 434.02 | 0.84 |
| | trifluoromethoxy-benzyl)-thiazolidin-4-one | | |
| 199 | 2-(4-Hydroxy-2-methoxy-phenyl)-3-(4- | 400.05 | 0.72 |
| | trifluoromethoxy-benzyl)-thiazolidin-4-one | | |
| 200 | 2-Benzo[1,3]dioxol-4-yl-3-(4-trifluoromethoxy- | 398.27 | 0.78 |
| | benzyl)-thiazolidin-4-one | | |
| 201 | 2-(2-Methoxy-5-methyl-phenyl)-3-(4- | 398.08 | 0.83 |
| | trifluoromethoxy-benzyl)-thiazolidin-4-one | | |
| 202 | 2-(6-Methoxy-indan-5-yl)-3-(4-trifluoromethoxy- | 424 | 0.86 |
| | benzyl)-thiazolidin-4-one | | |
| 203 | 3-Biphenyl-4-ylmethyl-2-(2,6-dimethoxy-phenyl)- | 406.03 | 1 |
| | thiazolidin-4-one | | |
| 204 | 2-(6-Methoxy-quinolin-5-yl)-3-(4-trifluoromethoxy- | 435.03 | 0.88 |
| | benzyl)-thiazolidin-4-one | | |
| 205 | 2-(2,6-Dimethoxy-phenyl)-3-(6-ethoxy-pyridin-3- | 375.1 | 0.85 |
| | ylmethyl)-thiazolidin-4-one | | |
| 206 | 2-(2,6-Dimethoxy-phenyl)-3-(5-ethoxy-pyridin-2- | 375.95 | 0.82 |
| | ylmethyl)-thiazolidin-4-one | | |
| 207 | 2-(2,6-Dimethoxy-phenyl)-3-(6-ethoxy-pyridazin-3- | 375.95 | 0.87 |
| | ylmethyl)-thiazolidin-4-one | | |
| 208 | 2-(2,3-Dihydro-benzo[1,4]dioxin-5-yl)-3-(4- | 412.03 | 0.79 |
| | trifluoromethoxy-benzyl)-thiazolidin-4-one | | |
| 209 | 3-Benzyl-2-(1H-indol-2-yl)-thiazolidin-4-one | 309.14 | 0.72 |
| 210 | 2-(2-Ethoxy-pyridin-3-yl)-3-(4-trifluoromethoxy- | 398.95 | 1.04 |
| | benzyl)-thiazolidin-4-one | | |
| 211 | 2-(1-Methyl-1H-indazol-3-yl)-3-(4-trifluoromethoxy- | 408.03 | 0.77 |
| | benzyl)-thiazolidin-4-one | | |
| 212 | 2-(6-Methoxy-pyridin-2-yl)-3-(4-trifluoromethoxy- | 385.04 | 0.78 |
| | benzyl)-thiazolidin-4-one | | |
| 213 | 3-(4-Ethoxy-benzyl)-2-(2-ethoxy-pyridin-3-yl)- | 359.01 | 1 |
| | thiazolidin-4-one | | |
| 214 | 2-(2-Methoxy-pyridin-3-yl)-3-(4-trifluoromethoxy- | 384.94 | 1.02 |
| | benzyl)-thiazolidin-4-on | | |
| 215 | 2-(4,6-Dimethoxy-pyrimidin-5-yl)-3-(4-ethoxy- | 376 | 0.96 |
| | benzyl)-thiazolidin-4-one | | |
| 216 | 3-Benzyl-2-(6-methoxy-pyridin-2-yl)-thiazolidin-4- | 301.14 | 0.69 |
| | one | | |
| 217 | 2-(4,6-Dimethoxy-pyrimidin-5-yl)-3-(4- | 415.84 | 1.02 |
| | trifluoromethoxy-benzyl)-thiazolidin-4-one | | |
| 218 | 3-Naphthalen-2-ylmethyl-2,3-dihydro- | 327.07 | 0.7 |
| | [2,2′]bithiazolyl-4-one | | |
| 219 | 3-Benzyl-2-(2-methoxy-phenyl)-thiazolidin-4-one | 300.25 | 0.72 |
| 220 | 3-Benzyl-2-(1-methyl-1H-indol-3-yl)-thiazolidin-4- | 323.17 | 0.74 |
| | one | | |
| 221 | 2-(2,6-Dimethoxy-phenyl)-3-thiophen-2-ylmethyl- | 336.14 | 0.71 |
| | thiazolidin-4-one | | |
| 222 | 3-Thiophen-2-ylmethyl-2-(2-trifluoromethyl-phenyl)- | 344.02 | 0.76 |
| | thiazolidin-4-one | | |
| 223 | 2-(2,6-Dimethoxy-phenyl)-5-methyl-3-quinoxalin-2- | 396.12 | 0.89 |
| | ylmethyl-thiazolidin-4-one | | |
| 224 | 2-(2,6-Dimethoxy-phenyl)-3-quinoxalin-2-ylmethyl- | 382.1 | 0.84 |
| | thiazolidin-4-one | | |
| 225 | 3-Benzo[b]thiophen-6-ylmethyl-2-(2,6-dimethoxy- | 400.48 | 1.1 |
| | phenyl)-5-methyl-thiazolidin-4-one | | |
| 226 | 2-(2,6-Dimethoxy-phenyl)-5-methyl-3-(1-methyl- | 397.54 | 1.12 |
| | 1H-indol-2-ylmethyl)-thiazolidin-4-one | | |
| 227 | 2-(2,6-Dimethoxy-phenyl)-5-methyl-3-(1-methyl- | 397.56 | 1.03 |
| | 1H-indol-6-ylmethyl)-thiazolidin-4-one | | |
| 228 | 3-Benzo[b]thiophen-5-ylmethyl-2-(2,6-dimethoxy- | 386.43 | 1.03 |
| | phenyl)-thiazolidin-4-one | | |
| 229 | 2-(2,6-Dimethoxy-phenyl)-5-methyl-3-(1-methyl- | 398.58 | 0.66 |
| | 1H-benzoimidazol-2-ylmethyl)-thiazolidin-4-one | | |
| 230 | 2-(2,6-Dimethoxy-phenyl)-5-methyl-3-quinolin-6- | 395.54 | 0.72 |
| | ylmethyl-thiazolidin-4-one | | |
| 231 | 3-Benzo[b]thiophen-2-ylmethyl-2-(2,6-dimethoxy- | 400.5 | 1.12 |
| | phenyl)-5-methyl-thiazolidin-4-one | | |
| 232 | 3-Benzo[b]thiophen-3-ylmethyl-2-(2,6-dimethoxy- | 400.5 | 1.11 |
| | phenyl)-5-methyl-thiazolidin-4-one | | |
| 233 | 2-(2,6-Dimethoxy-phenyl)-3-(1H-indol-2-ylmethyl)- | 383.55 | 1.05 |
| | 5-methyl-thiazolidin-4-one | | |
| 234 | 2-(2,6-Dimethoxy-phenyl)-5-methyl-3-quinolin-7- | 395.54 | 0.82 |
| | ylmethyl-thiazolidin-4-one | | |
| 235 | 3-Benzothiazol-2-ylmethyl-2-(2,6-dimethoxy- | 401.51 | 1.02 |
| | phenyl)-5-methyl-thiazolidin-4-one | | |
| 236 | 2-(2,6-Dimethoxy-phenyl)-5-methyl-3-quinoxalin-6- | 396.52 | 0.83 |
| | ylmethyl-thiazolidin-4-one | | |
| 237 | 3-Benzofuran-2-ylmethyl-2-(2,6-dimethoxy-phenyl)- | 384.55 | 1.08 |
| | 5-methyl-thiazolidin-4-one | | |
| 238 | 2-(2,6-Dimethoxy-phenyl)-3-(1H-indol-3-ylmethyl)- | 383.55 | 0.94 |
| | 5-methyl-thiazolidin-4-one | | |
| 239 | 2-(2,6-Dimethoxy-phenyl)-3-quinoxalin-6-ylmethyl- | 382.52 | 0.76 |
| | thiazolidin-4-one | | |
| 240 | 2-(3-Fluoro-2,6-dimethoxy-phenyl)-5-methyl-3-(4- | 446.41 | 1.18 |
| | trifluoromethoxy-benzyl)-thiazolidin-4-one | | |
| 241 | 2-(2,6-Dimethoxy-phenyl)-3-(1-methyl-1H-indol-2- | 383.54 | 1.05 |
| | ylmethyl)-thiazolidin-4-one | | |
| 242 | 3-Benzo[b]thiophen-2-ylmethyl-2-(2,6-dimethoxy- | 386.49 | 1.06 |
| | phenyl)-thiazolidin-4-one | | |
| 243 | 2-(2,6-Dimethoxy-phenyl)-3-(1-methyl-1H-indol-6- | 383.57 | 0.97 |
| | ylmethyl)-thiazolidin-4-one | | |
| 244 | 3-(4-Trifluoromethoxy-benzyl)-2-(2,4,6-trimethoxy- | 444.16 | 1.09 |
| | phenyl)-thiazolidin-4-one | | |
| 245 | 2-(3-Fluoro-2,6-dimethoxy-phenyl)-3-(4- | 432.44 | 1.11 |
| | trifluoromethoxy-benzyl)-thiazolidin-4-one | | |
| 246 | 5-Methyl-3-(4-trifluoromethoxy-benzyl)-2-(2,3,6- | 458.53 | 1.16 |
| | trimethoxy-phenyl)-thiazolidin-4-one | | |
| 247 | 2-(2,6-Dimethoxy-3-methyl-phenyl)-5-methyl-3-(4- | 442.6 | 1.21 |
| | trifluoromethoxy-benzyl)-thiazolidin-4-one | | |
| 248 | 3-Benzo[b]thiophen-3-ylmethyl-2-(2,6-dimethoxy- | 386.49 | 1.05 |
| | phenyl)-thiazolidin-4-one | | |
| 249 | 5-Methyl-3-(4-trifluoromethoxy-benzyl)-2-(2,4,6- | 383.55 | 0.94 |
| | trimethoxy-phenyl)-thiazolidin-4-one | | |
| 250 | 2-(2,6-Dimethoxy-3-methyl-phenyl)-3-(4- | 428.46 | 1.15 |
| | trifluoromethoxy-benzyl)-thiazolidin-4-one | | |
| 251 | 2-(2,6-Dimethoxy-phenyl)-3-quinolin-7-ylmethyl- | 381.5 | 0.74 |
| | thiazolidin-4-one | | |
| 252 | 2-(2,6-Dimethoxy-phenyl)-3-(1H-indol-2-ylmethyl)- | 369.53 | 0.99 |
| | thiazolidin-4-one | | |
| 253 | 2-(2,6-Dimethoxy-phenyl)-3-(1-methyl-1H- | 384.56 | 0.59 |
| | benzoimidazol-2-ylmethyl)-thiazolidin-4-one | | |
| 254 | 3-(1H-Benzoimidazol-2-ylmethyl)-2-(4-benzyloxy-3- | 446.46 | 0.71 |
| | methoxy-phenyl)-thiazolidin-4-one | | |
| 255 | 2-(2,6-Dimethoxy-phenyl)-3-quinolin-6-ylmethyl- | 381.49 | 0.65 |
| | thiazolidin-4-one | | |
| 256 | 3-Benzothiazol-2-ylmethyl-2-(2,6-dimethoxy- | 387.48 | 0.94 |
| | phenyl)-thiazolidin-4-one | | |
| 257 | 2-(3-Chloro-2,6-dimethoxy-phenyl)-5-methyl-3-(4- | 462.4 | 1.23 |
| | trifluoromethoxy-benzyl)-thiazolidin-4-one | | |
| 258 | 3-(4-Trifluoromethoxy-benzyl)-2-(2,3,6-trimethoxy- | 444.18 | 1.09 |
| | phenyl)-thiazolidin-4-one | | |
| 259 | 3-Benzofuran-2-ylmethyl-2-(2,6-dimethoxy-phenyl)- | 370.5 | 1.01 |
| | thiazolidin-4-one | | |
| 260 | 2-(2,6-Dimethoxy-phenyl)-5-methyl-3-(1-methyl- | 399.56 | 0.82 |
| | 1H-benzotriazol-5-ylmethyl)-thiazolidin-4-one | | |
| 261 | 3-(1H-Benzoimidazol-2-ylmethyl)-2-(2,6-dimethoxy- | 370.54 | 0.55 |
| | phenyl)-thiazolidin-4-one | | |
| 262 | 2-(3-Chloro-2,6-dimethoxy-phenyl)-3-(4- | 448.36 | 1.16 |
| | trifluoromethoxy-benzyl)-thiazolidin-4-one | | |
| 263 | 2-(2,6-Dimethoxy-phenyl)-3-(1H-indol-3-ylmethyl)- | 369.54 | 0.88 |
| | thiazolidin-4-one | | |
| 264 | 2-(2-Fluoro-4,6-dimethoxy-phenyl)-3-(4- | 432.45 | 1.11 |
| | trifluoromethoxy-benzyl)-thiazolidin-4-one | | |
| 265 | 3-(1H-Benzoimidazol-2-ylmethyl)-2-(4-benzyloxy-3- | 460.45 | 0.77 |
| | methoxy-phenyl)-5-methyl-thiazolidin-4-one | | |
| 266 | 2-(2-Chloro-4,6-dimethoxy-phenyl)-3-(4- | 448.4 | 1.15 |
| | trifluoromethoxy-benzyl)-thiazolidin-4-one | | |

### A.2 Preparation of 2-(2-hydroxy-6-methoxy-phenyl)-thiazolidin-4-one Intermediates (General Procedure)

A mixture of 2-hydroxy-6-methoxybenzaldehyde (3 mmol) and the respective amine R³—CH₂NH₂ (3 mmol, 1 eq) in dry DMF (7.3 mL) was stirred at RT for 20 min. followed by the addition of the respective mercapto-acid (9 mmol, 3 eq). After 30 min. of stirring at RT, DIPEA (6 mmol, 2 eq) and HBTU (6 mmol, 2 eq) were added and the stirring at RT was continued overnight. The reaction mixture was then diluted with EtOAc, washed with 5% aq. citric acid, water, sat. aq. NaHCO₃, brine, dried over MgSO₄, filtered and concentrated under reduced pressure to yield a crude solid. The products were purified by prep. HPLC to provide the desired intermediates.

### 2-(2-Hydroxy-6-methoxy-phenyl)-3-(4-trifluoromethoxy-benzyl)-thiazolidin-4-one

prepared by reaction of 2-hydroxy-6-methoxybenzaldehyde with 4-trifluoromethoxy-benzylamine; LC-MS: t_{R}=0.99 min; [M+H]⁺=399.94.

### 2-(2-Hydroxy-6-methoxy-phenyl)-3-(4-isopropoxy-benzyl)-thiazolidin-4-one

prepared by reaction of 2-hydroxy-6-methoxybenzaldehyde with 4-isopropoxy-benzylamine; LC-MS: t_{R}=0.70 min; [M+H]⁺=374.07.

### 3(4-Ethoxy-benzyl)-2-(2-hydroxy-6-methoxy-phenyl)-thiazolidin-4-one

prepared by reaction of 2-hydroxy-6-methoxybenzaldehyde with 4-ethoxy-benzylamine; LC-MS: t_{R}=0.67 min; [M+H]⁺=360.04.

### A.2.1 Preparation of Examples from 2-(2-hydroxy-6-methoxy-phenyl)-thiazolidin-4-one Intermediates (General Procedure)

To a cold (0° C.) mixture of the respective 2-(2-hydroxy-6-methoxy-phenyl)-thiazolidinone derivative (0.1 mmol) and dry K₂CO₃ (0.4 mmol, 4 eq) in dry DMF (0.5 mL) was added dropwise a solution of the respective alkylation agent (2-bromo-ethanol; 2-methoxy-ethylbromide; 1-chloro-2,3-dihydroxypropane; 2-hydroxy-propylbromide) (0.5 mmol, 5 eq) in dry DMF (0.1 mL). The reaction mixture was stirred at 90° C. for 12 h. After cooling to RT, the reaction mixture was filtered and directly purified by prep. HPLC to provide the final compound.

The following Examples were synthesized according to the above general procedure:

| Example | Name | [M + H]⁺ | t_{R} |
|---|---|---|---|
| 267 | 2-[2-(2-Hydroxy-ethoxy)-6-methoxy- | 443.99 | 0.99 |
| | phenyl]-3-(4-trifluoromethoxy-benzyl)- | | |
| | thiazolidin-4-one | | |
| 268 | 2-[2-(2-Hydroxy-ethoxy)-6-methoxy- | 404.05 | 0.68 |
| | phenyl]-3-(4-trifluoromethoxy-benzyl)- | | |
| | thiazolidin-4-one | | |
| 269 | 2-[2-(2-Hydroxy-ethoxy)-6-methoxy- | 417.07 | 0.71 |
| | phenyl]-3-(4-isopropoxy-benzyl)- | | |
| | thiazolidin-4-on | | |

The following further Examples were synthesized according to the above general procedure:

| Example | Name | [M + H]⁺ | t_{R} |
|---|---|---|---|
| 270 | 2-[2-(S)-(2,3-Dihydroxy-propoxy)-6- | 473.86 | 0.92 |
| | methoxy-phenyl]-3-(4-trifluoromethoxy- | | |
| | benzyl)-thiazolidin-4-one | | |
| 271 | 2-[2-(R)-(2,3-Dihydroxy-propoxy)-6- | 473.89 | 0.92 |
| | methoxy-phenyl]-3-(4-trifluoromethoxy- | | |
| | benzyl)-thiazolidin-4-one | | |
| 272 | 2-[2-Methoxy-6-(2-methoxy- | 457.88 | 1.06 |
| | ethoxy)-phenyl]-3-(4-trifluoromethoxy- | | |
| | benzyl)-thiazolidin-4-one | | |
| 273 | 2-[2-(2-Hydroxy-propoxy)-6- | 458.1 | 0.75 |
| | methoxy-phenyl]-3-(4-trifluoromethoxy- | | |
| | benzyl)-thiazolidin-4-one | | |
| 274 | 3-(4-Ethoxy-benzyl)-2-[2-(2-hydroxy- | 417.68 | 0.7 |
| | propoxy)-6-methoxy-phenyl]-thiazolidin- | | |
| | 4-one | | |
| 275 | 2-[2-(2-Hydroxy-propoxy)-6- | 432.14 | 0.73 |
| | methoxy-phenyl]-3-(4-isopropoxy- | | |
| | benzyl)-thiazolidin-4-one | | |

### A.3 Preparation of 2-(2,6-Dimethoxy-phenyl)-3-(4-hydroxy-benzyl)-thiazolidin-4-one

A mixture of 2,6-dimethoxybenzaldehyde (3 mmol) and 4-hydroxy-benzyl amine (3 mmol, 1 eq) in dry DMF (7.3 mL) was stirred at RT for 20 min. followed by the addition of the respective mercapto-acid (9 mmol, 3 eq). After 30 min. of stirring at RT, DIPEA (6 mmol, 2 eq) and HBTU (6 mmol, 2 eq) were added and the stirring at RT was continued overnight. The reaction mixture was then diluted with EtOAc, washed with 5% aq. citric acid, water, sat. aq. NaHCO₃, brine, dried over MgSO₄, filtered and concentrated under reduced pressure to yield a crude solid. The product was purified by prep. HPLC to provide the final compound.

LC-MS: t_{R}=0.88 min; [M+H]⁺=345.96

### A.3.1 Preparation of Examples from 2-(2,6-Dimethoxy-phenyl)-3-(4-hydroxy-benzyl)-thiazolidin-4-one (General Procedure)

A mixture of 2-(2,6-Dimethoxy-phenyl)-3-(4-hydroxy-benzyl)-thiazolidin-4-one (0.15 mmol, prepared according to the general method described in A.1), the respective Ar³-chloride or Ar³-bromide (0.15 mmol), dry CuCl (0.075 mmol, 0.5 eq), Cs₂CO₃ (0.15 mmol), 2,2,6,6-tetramethyl-heptane-3,5-dione (0.15 mmol) in NMP (0.9 mL) was stirred at 140° C. overnight. After cooling to RT, the reaction mixture was filtered and directly purified by prep. HPLC to provide the final compound.

The following Examples were synthesized according to the above general procedure:

| Example | Name | [M + H]⁺ | t_{R} |
|---|---|---|---|
| 276 | 2-(2,6-Dimethoxy-phenyl)-3-[4- | 424.11 | 0.72 |
| | (pyrimidin-5-yloxy)-benzyl]-thiazolidin-4- | | |
| | one | | |
| 277 | 2-(2,6-Dimethoxy-phenyl)-3-[4- | 423.14 | 0.74 |
| | (pyridin-2-yloxy)-benzyl]-thiazolidin-4- | | |
| | one | | |
| 278 | 2-(2,6-Dimethoxy-phenyl)-3-[4- | 423.05 | 0.63 |
| | (pyridin-3-yloxy)-benzyl]-thiazolidin-4- | | |
| | one | | |
| 279 | 2-(2,6-Dimethoxy-phenyl)-3-[4- | 424.14 | 0.73 |
| | (pyrazin-2-yloxy)-benzyl]-thiazolidin-4- | | |
| | one | | |
| 280 | 2-(2,6-Dimethoxy-phenyl)-3-[4- | 438.19 | 0.66 |
| | (6-methyl-pyridazin-3-yloxy)-benzyl]- | | |
| | thiazolidin-4-one | | |
| 281 | 2-(2,6-Dimethoxy-phenyl)-3-[4- | 437.05 | 0.95 |
| | (5-methyl-pyridin-2-yloxy)-benzyl]- | | |
| | thiazolidin-4-one | | |
| 282 | 2-(2,6-Dimethoxy-phenyl)-3-[4- | 453.04 | 0.97 |
| | (6-methoxy-pyridin-2-yloxy)-benzyl]- | | |
| | thiazolidin-4-one | | |
| 283 | 2-(2,6-Dimethoxy-phenyl)-3-[4- | 437.05 | 0.94 |
| | (6-methyl-pyridin-2-yloxy)-benzyl]- | | |
| | thiazolidin-4-one | | |
| 284 | 2-(2,6-Dimethoxy-phenyl)-3-[4- | 437.05 | 0.94 |
| | (4-methyl-pyridin-2-yloxy)-benzyl]- | | |
| | thiazolidin-4-one | | |
| 285 | 2-(2,6-Dimethoxy-phenyl)-3-[4- | 437.05 | 0.96 |
| | (3-methyl-pyridin-2-yloxy)-benzyl]- | | |
| | thiazolidin-4-one | | |
| 286 | 2-(2,6-Dimethoxy-phenyl)-3-[4- | 453.06 | 0.91 |
| | (5-methoxy-pyridin-2-yloxy)-benzyl]- | | |
| | thiazolidin-4-one | | |

The following further Example was synthesized according to the above general procedure:

| Example | Name | [M + H]⁺ | t_{R} |
|---|---|---|---|
| 287 | 2-(2,6-Dimethoxy-phenyl)-3-[4-(6- | 490.92 | 0.98 |
| | trifluoromethyl-pyridin-2-yloxy)-benzyl]- | | |
| | thiazolidin-4-one | | |

### A.4 Synthesis of 1-oxo-thiazolidin-4-one and 1,1-dioxo-thiazolidin-4-one derivatives

### A.4.1 Synthesis of 1,1-oxo-thiazolidin-4-one derivatives (X is SO₂) (General Procedure)

To a cold (0° C.) solution of the respective thiazolidin-4-one derivative (1 eq) in dry DCM (1.2 mL/0.15 mmol) was added mCPBA (4 eq). The reaction mixture was stirred at 0° C. for 1 h, then at RT overnight. Then a sat. aq. NaHCO₃ solution was added and the reaction mixture was extracted with DCM. The combined organic extracts were dried over MgSO₄, filtered and concentrated under reduced pressure to yield a crude solid. The products were purified by prep. HPLC to provide the final compound.

The following Examples were synthesized according to the above general procedure:

| Example | Name | [M + H]⁺ | t_{R} |
|---|---|---|---|
| 288 | 2-(2,6-Dimethoxy-phenyl)-1,1-dioxo-3-(4- | 446.01 | 0.75 |
| | trifluoromethoxy-benzyl)-thiazolidin-4-one | | |
| 289 | 2-(2,6-Dimethoxy-phenyl)-3-(4- | 420.11 | 0.73 |
| | isopropoxy-benzyl)-1,1-dioxo-thiazolidin- | | |
| | 4-one | | |
| 290 | 2-(2,6-Dimethoxy-phenyl)-3-(4-ethoxy- | 406.17 | 0.70 |
| | benzyl)-1,1-dioxo-thiazolidin-4-one | | |

### A.4.2 Synthesis of 1-oxo-thiazolidin-4-one derivatives (X is S(O)) (General Procedure)

To a cold (0° C.) solution of the respective thiazolidin-4-one derivative (1 eq) in dry DCM (1.2 mL/0.15 mmol) was added mCPBA (1.1 eq). The reaction mixture was stirred at 0° C. for 1 h, then sat. aq. NaHCO₃ solution was added and the reaction mixture was extracted with DCM. The combined organic extracts were dried over MgSO₄, filtered and concentrated under reduced pressure to yield a crude solid. The products were purified by prep. HPLC to provide the final compound.

The following Example was synthesized according to the above general procedure:

| Example | Name | [M + H]⁺ | t_{R} |
|---|---|---|---|
| 291 | 2-(2,6-Dimethoxy-phenyl)-1-oxo-3-(4- | 430.02 | 0.71 |
| | trifluoromethoxy-benzyl)-thiazolidin-4-one | | |

### II—Biological Assays

### In Vitro Assay

The orexin receptor antagonistic activity of the compounds of formula (I) is determined in accordance with the following experimental method.

Chinese hamster ovary (CHO) cells expressing the human orexin-1 receptor and the human orexin-2 receptor, respectively, are grown in culture medium (Ham F-12 with L-Glutamine) containing 300 μg/ml G418, 100 U/ml penicillin, 100 μg/ml streptomycin and 10% heat inactivated fetal calf serum (FCS). The cells are seeded at 20′000 cells/well into 384-well black clear bottom sterile plates (Greiner). The seeded plates are incubated overnight at 37° C. in 5% CO₂.

Human orexin-A as an agonist is prepared as 1 mM stock solution in MeOH:water (1:1), diluted in HBSS containing 0.1% bovine serum albumin (BSA), NaHCO₃: 0.375 g/l and 20 mM HEPES for use in the assay at a final concentration of 3 nM.

Antagonists are prepared as 10 mM stock solution in DMSO, then diluted in 384-well plates using DMSO followed by a transfer of the dilutions into in HBSS containing 0.1% bovine serum albumin (BSA), NaHCO₃: 0.375 g/l and 20 mM HEPES. On the day of the assay, 50 μl of staining buffer (HBSS containing 1% FCS, 20 mM HEPES, NaHCO₃: 0.375 g/l, 5 mM probenecid (Sigma) and 3 μM of the fluorescent calcium indicator fluo-4 AM (1 mM stock solution in DMSO, containing 10% pluronic) is added to each well. The 384-well cell-plates are incubated for 50 min at 37° C. in 5% CO₂ followed by equilibration at rt for 30-120 min before measurement.

Within the Fluorescent Imaging Plate Reader (FLIPR Tetra, Molecular Devices), antagonists are added to the plate in a volume of 10 μl/well, incubated for 10 min and finally 10 μl/well of agonist is added. Fluorescence is measured for each well at 1 second intervals, and the height of each fluorescence peak is compared to the height of the fluorescence peak induced by 3 nM orexin-A with vehicle in place of antagonist. For each antagonist, the IC₅₀ value (the concentration of compound needed to inhibit 50% of the agonistic response) is determined and normalized using the obtained IC₅₀ value of a on-plate reference compound. Optimized conditions were achieved by adjustment of pipetting speed and cell splitting regime. The calculated IC₅₀ values of the compounds may fluctuate depending on the daily cellular assay performance. Fluctuations of this kind are known to those skilled in the art.

Antagonistic activities (IC₅₀ values) of all exemplified compounds are below 10000 nM with respect to the OX₁ and/or the OX₂ receptor. Antagonistic activities (IC₅₀ values) of 271 exemplified compounds are in the range of 11-9999 nM with an average of 1930 nM with respect to the OX₁ receptor; 20 compounds have been measured with an IC₅₀ value higher than the limits of detection in this assay. IC₅₀ values of all exemplified compounds are in the range of 6-8064 nM with an average of 315 nM with respect to the OX₂ receptor. Antagonistic activities of selected compounds are displayed in Table 1.

TABLE 1

| | OX₁ IC₅₀ | OX₂ IC₅₀ | | OX₁ IC₅₀ | OX₂ IC₅₀ | | OX₁ IC₅₀ | OX₂ IC₅₀ |
|---|---|---|---|---|---|---|---|---|
| Ex. | [nM] | [nM] | Ex. | [nM] | [nM] | Ex. | [nM] | [nM] |
| 2 | 144 *⁴ | 15 *⁴ | 25 | 561 | 16 | 81 | 2285 *² | 13 *² |
| 4 | 377 | 27 | 28 | 208 *² | 16 *² | 101 | 488 | 51 |
| 12 | 310 | 11 | 29 | 1045 | 36 | 119 | 355 | 61 |
| 24 | 278 | 14 | 30 | 356 | 442 | 139 | 440 *⁴ | 30 *⁴ |
| 26 | 4296 | 126 | 32 | 15 | 6 | 175 | 86 | 663 |
| 27 | 154 | 18 | 33 | 105 | 8 | 176 | 20 *² | 295 *² |
| 31 | 11 | 30 | 34 | 107 | 28 | 203 | 1826 | 125 |
| 35 | 170 | 130 | 36 | 171 | 33 | 265 | 2632 | 533 |
| 40 | 1312 | 68 | 268 | 1836 | 45 | 267 | 263 | 24 |
| 43 | 793 | 70 | 269 | 2065 | 25 | 278 | 1031 | 10 |
| 65 | 1427 | 21 | 276 | 1591 | 121 | 283 | 942 | 51 |
| 72 | 778 | 115 | 277 | 411 *⁴ | 8 *⁴ | 289 | 181 | 16 |
| 1 | 309 | 104 | 279 | 1424 | 32 | 14 | 31 *⁴ | 44 *⁴ |
| 3 | 1424 | 120 | 280 | 1744 | 121 | 15 | 853 | 48 |
| 5 | 169 *⁴ | 16 *⁴ | 281 | 781 | 61 | 16 | 454 | 24 |
| 6 | 140 *⁴ | 24 *⁴ | 282 | 1955 | 127 | 17 | 363 | 15 |
| 7 | 244 *² | 13 *² | 284 | 344 | 7 | 18 | 323 *³ | 10 *³ |
| 8 | 83 *² | 23 *² | 285 | 1219 | 135 | 19 | 8124 | 153 |
| 9 | 326 | 39 | 286 | 553 | 52 | 20 | 1560 | 342 |
| 10 | 144 | 16 | 288 | 166 *⁵ | 6 *⁵ | 21 | 1446 | 60 |
| 11 | 885 | 46 | 290 | 375 | 21 | 22 | 2362 | 19 |
| 13 | 244 | 43 | 23 | 204 | 28 | | | |

Ex. = Example of compound
*² geometric mean from n = 2 values;
*³ geometric mean from n = 3 values
*⁴ geometric mean from n = 4 values
*⁵ geometric mean from n = 5 values

1. A compound, in a free or a pharmaceutically acceptable salt form, for the prevention or treatment of diseases related to the orexin system; wherein said compound is a compound of the formula (I) 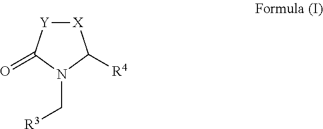
wherein
X represents S, S(O), or SO₂;
Y represents CH₂, CH₂CH₂, CHR¹, or CR¹R²; wherein
R¹ and R² independently represent (C₁₋₄)alkyl;
R³ represents Ar¹ or Ar³—Z—Ar²-* wherein the asterisk indicates the bond that is attached to the rest of the molecule; wherein
Ar¹ represents aryl or heteroaryl, wherein the aryl or heteroaryl is independently unsubstituted, or mono-, di-, or tri-substituted, wherein the substituents are independently selected from the group consisting of (C₁₋₄)alkyl, (C₁₋₄)alkoxy, hydroxy-(C₁₋₄)alkoxy, halogen, (C₁₋₃)fluoroalkyl, (C₁₋₃)fluoroalkoxy, (C₁₋₃)fluoroalkyl-thio-, hydroxy-(C₁₋₄)alkoxy, (C₁₋₄)alkoxy-(C₁₋₄)alkoxy, and —NR⁵R⁶;
Ar² represents phenyl or 5- to 6-membered heteroaryl;
Z represents a bond, O, or —CH₂—O-* wherein the asterisk indicates the bond that is attached to Ar²;
Ar³ represents phenyl or 5- to 6-membered heteroaryl wherein the phenyl or 5- to 6-membered heteroaryl is independently unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy;
R⁴ represents aryl or heteroaryl, wherein the aryl or heteroaryl is independently unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, hydroxy-(C₁₋₄)alkoxy, dihydroxy-(C₁₋₄)alkoxy, (C₁₋₄)alkoxy-(C₁₋₄)alkoxy, hydroxy, benzyloxy, and benzyl; and
R⁵ and R⁶ together with the nitrogen to which they are attached form an azetidinyl, a pyrrolidinyl, or a piperidinyl ring.

2. The compound according to claim 1, in a free or a pharmaceutically acceptable salt form, for the prevention or treatment of diseases related to the orexin system; wherein X represents S.

3. The compound according to claim 1, in a free or a pharmaceutically acceptable salt form, for the prevention or treatment of diseases related to the orexin system; wherein Y represents CH₂, CHR¹, or CR¹R².

4. The compound according to claim 1, in a free or a pharmaceutically acceptable salt form, for the prevention or treatment of diseases related to the orexin system; wherein
Ar¹ represents aryl which is unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy.

5. The compound according to claim 1, in a free or a pharmaceutically acceptable salt form, for the prevention or treatment of diseases related to the orexin system; wherein
Ar² represents phenyl;
Z represents O; and
Ar³ represents phenyl which is unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy; or
Ar³ represents 5- to 6-membered heteroaryl which is unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy.

6. The compound according to claim 1, in a free or a pharmaceutically acceptable salt form, for the prevention or treatment of diseases related to the orexin system; wherein
R⁴ represents aryl which is unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, hydroxy-(C₁₋₄)alkoxy, dihydroxy-(C₁₋₄)alkoxy, (C₁₋₄)alkoxy-(C₁₋₄)alkoxy, hydroxy, benzyloxy, and benzyl; or
R⁴ represents heteroaryl which is unsubstituted, or mono-, di-, or tri-substituted, wherein the substituents are independently selected from the group consisting of (C₁₋₄)alkyl, (C₁₋₄)alkoxy, halogen, (C₁₋₃)fluoroalkyl, and (C₁₋₃)fluoroalkoxy.

7. The compound according to claim 1, in a free or a pharmaceutically acceptable salt form, for the prevention or treatment of diseases related to the orexin system; wherein R⁴ is at least mono-substituted, wherein said substituent is attached in ortho position to the point of attachment of R⁴ to the rest of the molecule.

8. The compound according to claim 1, in a free or a pharmaceutically acceptable salt form, for the prevention or treatment of diseases related to the orexin system; wherein the compound is selected from the group consisting of:
3-Benzo[1,3]dioxol-5-ylmethyl-2-(2-ethoxy-phenyl)-[1,3]thiazinan-4-one;
2-(2-Ethoxy-phenyl)-3-(4-methoxy-benzyl)-[1,3]thiazinan-4-one;
3-(2-Ethoxy-benzyl)-2-(2-ethoxy-phenyl)-[1,3]thiazinan-4-one;
3-(2-Difluoromethoxy-benzyl)-2-(2-ethoxy-phenyl)-[1,3]thiazinan-4-one;
2-(2-Ethoxy-phenyl)-3-(3-methyl-benzyl)-[1,3]thiazinan-4-one;
2-(2-Ethoxy-phenyl)-3-(3-fluoro-benzyl)-[1,3]thiazinan-4-one;
3-(2,6-Dimethoxy-benzyl)-2-(2-ethoxy-phenyl)-[1,3]thiazinan-4-one;
3-(2,3-Dihydro-benzo[1,4]dioxin-6-ylmethyl)-2-(2-ethoxy-phenyl)-[1,3]thiazinan-4-one;
3-(3-Chloro-benzyl)-2-(2-ethoxy-phenyl)-[1,3]thiazinan-4-one;
2-(2-Ethoxy-phenyl)-3-(3-methoxy-benzyl)-[1,3]thiazinan-4-one;
2-(2-Ethoxy-phenyl)-3-(2-fluoro-benzyl)-[1,3]thiazinan-4-one;
3-(4-Chloro-benzyl)-2-(2-ethoxy-phenyl)-[1,3]thiazinan-4-one;
3-(2,5-Difluoro-benzyl)-2-(2-ethoxy-phenyl)-[1,3]thiazinan-4-one;
2-(2-Ethoxy-phenyl)-3-(3-trifluoromethoxy-benzyl)-[1,3]thiazinan-4-one;
3-(2,5-Dimethoxy-benzyl)-2-(2-ethoxy-phenyl)-[1,3]thiazinan-4-one;
3-(3-Chloro-4-fluoro-benzyl)-2-(2-ethoxy-phenyl)-[1,3]thiazinan-4-one;
3-(2-Chloro-benzyl)-2-(2-ethoxy-phenyl)-[1,3]thiazinan-4-one;
2-(2-Ethoxy-phenyl)-3-(4-trifluoromethoxy-benzyl)-[1,3]thiazinan-4-one;
2-(2-Ethoxy-phenyl)-3-(2-methoxy-benzyl)-[1,3]thiazinan-4-one;
2-(2-Ethoxy-phenyl)-3-(2-methyl-benzyl)-[1,3]thiazinan-4-one;
3-(2,3-Dimethoxy-benzyl)-2-(2-ethoxy-phenyl)-[1,3]thiazinan-4-one;
2-(2-Methoxy-phenyl)-3-thiophen-2-ylmethyl-thiazolidin-4-one;
2-(2-Ethoxy-phenyl)-3-thiophen-2-ylmethyl-thiazolidin-4-one;
2-(2-Ethoxy-phenyl)-3-(2-fluoro-benzyl)-thiazolidin-4-one;
2-(2-Ethoxy-phenyl)-3-(4-methoxy-benzyl)-thiazolidin-4-one;
3-(2,5-Difluoro-benzyl)-2-(2-ethoxy-phenyl)-thiazolidin-4-one;
2-(2-Ethoxy-phenyl)-3-(3-fluoro-benzyl)-thiazolidin-4-one;
3-(2,6-Difluoro-benzyl)-2-(2-ethoxy-phenyl)-thiazolidin-4-one;
3-(3,4-Difluoro-benzyl)-2-(2-ethoxy-phenyl)-thiazolidin-4-one;
2-(2-Ethoxy-phenyl)-3-naphthalen-1-ylmethyl-thiazolidin-4-one;
3-(3,5-Difluoro-benzyl)-2-(2-ethoxy-phenyl)-thiazolidin-4-one;
3-(2,5-Difluoro-benzyl)-2-(2,6-dimethoxy-phenyl)-thiazolidin-4-one;
3-(3,5-Difluoro-benzyl)-2-(2,6-dimethoxy-phenyl)-thiazolidin-4-one;
3-Benzo[1,3]dioxol-5-ylmethyl-2-(2-ethoxy-phenyl)-thiazolidin-4-one;
3-(2,3-Dihydro-benzofuran-5-ylmethyl)-2-(2-ethoxy-phenyl)-thiazolidin-4-one;
3-(2,3-Dihydro-benzo[1,4]dioxin-6-ylmethyl)-2-(2-ethoxy-phenyl)-thiazolidin-4-one;
3-(4-Chloro-benzyl)-2-(2-ethoxy-phenyl)-thiazolidin-4-one;
2-(2-Ethoxy-phenyl)-3-(2-trifluoromethyl-benzyl)-thiazolidin-4-one;
2-(2-Ethoxy-phenyl)-3-(2-methoxy-benzyl)-thiazolidin-4-one;
2-(2-Ethoxy-phenyl)-3-(4-methyl-benzyl)-thiazolidin-4-one;
3-(2-Chloro-benzyl)-2-(2-ethoxy-phenyl)-thiazolidin-4-one;
2-(2-Ethoxy-phenyl)-3-(2-methyl-benzyl)-thiazolidin-4-one;
3-(2-Ethoxy-benzyl)-2-(2-ethoxy-phenyl)-thiazolidin-4-one;
3-(3-Chloro-benzyl)-2-(2-ethoxy-phenyl)-thiazolidin-4-one;
2-(2-Ethoxy-phenyl)-3-(3-trifluoromethoxy-benzyl)-thiazolidin-4-one;
2-(2-Ethoxy-phenyl)-3-(4-trifluoromethoxy-benzyl)-thiazolidin-4-one;
3-(2,3-Dimethoxy-benzyl)-2-(2-ethoxy-phenyl)-thiazolidin-4-one;
3-(2,4-Dichloro-benzyl)-2-(2-ethoxy-phenyl)-thiazolidin-4-one;
3-(3,4-Dichloro-benzyl)-2-(2-ethoxy-phenyl)-thiazolidin-4-one;
2-(2-Ethoxy-phenyl)-3-(3-methoxy-benzyl)-thiazolidin-4-one;
3-(2,5-Dimethoxy-benzyl)-2-(2-ethoxy-phenyl)-thiazolidin-4-one;
3-(2,4-Dimethoxy-benzyl)-2-(2,6-dimethoxy-phenyl)-thiazolidin-4-one;
2-(2-Ethoxy-phenyl)-3-(2-trifluoromethoxy-benzyl)-thiazolidin-4-one;
3-(2-Difluoromethoxy-benzyl)-2-(2-ethoxy-phenyl)-thiazolidin-4-one;
3-(2,6-Dimethoxy-benzyl)-2-(2-ethoxy-phenyl)-thiazolidin-4-one;
3-(3,4-Dimethoxy-benzyl)-2-(2-ethoxy-phenyl)-thiazolidin-4-one;
3-(3-Chloro-4-fluoro-benzyl)-2-(2-ethoxy-phenyl)-thiazolidin-4-one;
3-(2-Chloro-4-fluoro-benzyl)-2-(2-ethoxy-phenyl)-thiazolidin-4-one;
2-(2-Ethoxy-phenyl)-3-(3-fluoro-4-methyl-benzyl)-thiazolidin-4-one;
3-(5-Chloro-2-fluoro-benzyl)-2-(2-ethoxy-phenyl)-thiazolidin-4-one;
3-(4-Chloro-2-fluoro-benzyl)-2-(2-ethoxy-phenyl)-thiazolidin-4-one;
3-(4-tert-Butyl-benzyl)-2-(2-ethoxy-phenyl)-thiazolidin-4-one;
2-(2-Ethoxy-phenyl)-3-(2,4,6-trimethyl-benzyl)-thiazolidin-4-one;
3-Benzo[1,3]dioxol-5-ylmethyl-2-(2,6-dimethoxy-phenyl)-thiazolidin-4-one;
2-(2,6-Dimethoxy-phenyl)-3-(2-ethoxy-benzyl)-thiazolidin-4-one;
3-(2,4-Dichloro-benzyl)-2-(2,6-dimethoxy-phenyl)-thiazolidin-4-one;
3-(2-Difluoromethoxy-benzyl)-2-(2,6-dimethoxy-phenyl)-thiazolidin-4-one;
2-(2,6-Dimethoxy-phenyl)-3-(2,4,6-trimethyl-benzyl)-thiazolidin-4-one;
3-(2,3-Dihydro-benzofuran-5-ylmethyl)-2-(2,6-dimethoxy-phenyl)-thiazolidin-4-one;
2-(2,6-Dimethoxy-phenyl)-3-(3-methyl-benzyl)-thiazolidin-4-one;
2-(2,6-Dimethoxy-phenyl)-3-(3-fluoro-benzyl)-thiazolidin-4-one;
3-(3,4-Dichloro-benzyl)-2-(2,6-dimethoxy-phenyl)-thiazolidin-4-one;
3-(2,6-Dimethoxy-benzyl)-2-(2,6-dimethoxy-phenyl)-thiazolidin-4-one;
3-(3,4-Difluoro-benzyl)-2-(2,6-dimethoxy-phenyl)-thiazolidin-4-one;
3-(2,3-Dihydro-benzo[1,4]dioxin-6-ylmethyl)-2-(2,6-dimethoxy-phenyl)-thiazolidin-4-one;
2-(2,6-Dimethoxy-phenyl)-3-(4-methyl-benzyl)-thiazolidin-4-one;
3-(3-Chloro-benzyl)-2-(2,6-dimethoxy-phenyl)-thiazolidin-4-one;
2-(2,6-Dimethoxy-phenyl)-3-(3-methoxy-benzyl)-thiazolidin-4-one;
3-(3,4-Dimethoxy-benzyl)-2-(2,6-dimethoxy-phenyl)-thiazolidin-4-one;
2-(2,6-Dimethoxy-phenyl)-3-(3-fluoro-4-methyl-benzyl)-thiazolidin-4-one;
2-(2,6-Dimethoxy-phenyl)-3-(2-fluoro-benzyl)-thiazolidin-4-one;
3-(4-Chloro-benzyl)-2-(2,6-dimethoxy-phenyl)-thiazolidin-4-one;
2-(2,6-Dimethoxy-phenyl)-3-(3-trifluoromethoxy-benzyl)-thiazolidin-4-one;
3-(2,5-Dimethoxy-benzyl)-2-(2,6-dimethoxy-phenyl)-thiazolidin-4-one;
3-(3-Chloro-4-fluoro-benzyl)-2-(2,6-dimethoxy-phenyl)-thiazolidin-4-one;
3-(5-Chloro-2-fluoro-benzyl)-2-(2,6-dimethoxy-phenyl)-thiazolidin-4-one;
2-(2,6-Dimethoxy-phenyl)-3-(2-trifluoromethyl-benzyl)-thiazolidin-4-one;
3-(2-Chloro-benzyl)-2-(2,6-dimethoxy-phenyl)-thiazolidin-4-one;
3-(2,4-Dimethoxy-benzyl)-2-(2,6-dimethoxy-phenyl)-thiazolidin-4-one;
3-(2,6-Difluoro-benzyl)-2-(2,6-dimethoxy-phenyl)-thiazolidin-4-one;
3-(4-Chloro-2-fluoro-benzyl)-2-(2,6-dimethoxy-phenyl)-thiazolidin-4-one;
2-(2,6-Dimethoxy-phenyl)-3-(2-methoxy-benzyl)-thiazolidin-4-one;
2-(2,6-Dimethoxy-phenyl)-3-(2-methyl-benzyl)-thiazolidin-4-one;
2-(2,6-Dimethoxy-phenyl)-3-(2-trifluoromethoxy-benzyl)-thiazolidin-4-one;
3-(2-Chloro-4-fluoro-benzyl)-2-(2,6-dimethoxy-phenyl)-thiazolidin-4-one;
3-(2,2-Difluoro-benzo[1,3]dioxol-5-ylmethyl)-2-(2,6-dimethoxy-phenyl)-thiazolidin-4-one;
2-(2,6-Dimethoxy-phenyl)-3-(4-trifluoromethyl-benzyl)-thiazolidin-4-one;
2-(2,6-Dimethoxy-phenyl)-3-(4-pyrrol-1-yl-benzyl)-thiazolidin-4-one;
2-(2,6-Dimethoxy-phenyl)-3-(3-fluoro-4-trifluoromethyl-benzyl)-thiazolidin-4-one;
2-(2,6-Dimethoxy-phenyl)-3-(4-pyrrolidin-1-yl-benzyl)-thiazolidin-4-one;
2-(2,6-Dimethoxy-phenyl)-3-(4-pyrazol-1-yl-benzyl)-thiazolidin-4-one;
2-(2,6-Dimethoxy-phenyl)-3-(3,4,5-trifluoro-benzyl)-thiazolidin-4-one;
2-(2,6-Dimethoxy-phenyl)-3-(4-trifluoromethylsulfanyl-benzyl)-thiazolidin-4-one;
3-Benzyl-2-(2-ethoxy-phenyl)-thiazolidin-4-one;
2-(2-Fluoro-6-methoxy-phenyl)-3-naphthalen-2-ylmethyl-thiazolidin-4-one;
2-(2-Ethoxy-phenyl)-3-pyridin-2-ylmethyl-thiazolidin-4-one;
2-(2-Ethoxy-phenyl)-3-naphthalen-2-ylmethyl-thiazolidin-4-one;
2-(2-Ethoxy-phenyl)-3-pyridin-4-ylmethyl-thiazolidin-4-one;
2-(2-Ethoxy-phenyl)-3-(1-methyl-1H-pyrrol-2-ylmethyl)-thiazolidin-4-one;
2-(2,6-Difluoro-phenyl)-3-(4-trifluoromethoxy-benzyl)-thiazolidin-4-one;
2-(2,6-Difluoro-phenyl)-3-naphthalen-2-ylmethyl-thiazolidin-4-one;
2-(2-Methoxy-phenyl)-3-(4-trifluoromethoxy-benzyl)-thiazolidin-4-one;
2-(2-Methoxy-phenyl)-3-naphthalen-2-ylmethyl-thiazolidin-4-one;
2-(2,6-Dimethoxy-phenyl)-3-naphthalen-1-ylmethyl-thiazolidin-4-one;
3-Benzyl-2-(2,6-dimethoxy-phenyl)-thiazolidin-4-one;
2-(2-Fluoro-6-methoxy-phenyl)-3-naphthalen-1-ylmethyl-thiazolidin-4-one;
2-(2-Fluoro-6-methoxy-phenyl)-3-thiophen-2-ylmethyl-thiazolidin-4-one, 2-(2-Fluoro-6-methoxy-phenyl)-3-(4-trifluoromethoxy-benzyl)-thiazolidin-4-one;
3-Benzyl-2-(2-fluoro-6-methoxy-phenyl)-thiazolidin-4-one;
2-(2-Fluoro-6-methoxy-phenyl)-3-(1-methyl-1H-pyrrol-2-ylmethyl)-thiazolidin-4-one;
3-Benzyl-5-methyl-2-phenyl-thiazolidin-4-one;
3-(4-Ethoxy-benzyl)-5-methyl-2-phenyl-thiazolidin-4-one;
5-Methyl-3-naphthalen-2-ylmethyl-2-phenyl-thiazolidin-4-one;
3-Benzyl-2-(2-methoxy-phenyl)-5-methyl-thiazolidin-4-one;
3-(4-Ethoxy-benzyl)-2-(2-methoxy-phenyl)-5-methyl-thiazolidin-4-one;
2-(2-Methoxy-phenyl)-5-methyl-3-naphthalen-2-ylmethyl-thiazolidin-4-one;
3-Benzyl-2-(2-ethoxy-phenyl)-5-methyl-thiazolidin-4-one;
3-(4-Ethoxy-benzyl)-2-(2-ethoxy-phenyl)-5-methyl-thiazolidin-4-one;
2-(2-Ethoxy-phenyl)-5-methyl-3-naphthalen-2-ylmethyl-thiazolidin-4-one;
3-Benzyl-2-(2,6-dimethoxy-phenyl)-5-methyl-thiazolidin-4-one;
2-(2,6-Dimethoxy-phenyl)-5-methyl-3-(4-trifluoromethyl-benzyl)-thiazolidin-4-one;
3-Benzyl-2-(2-methoxy-phenyl)-5,5-dimethyl-thiazolidin-4-one;
3-(4-Ethoxy-benzyl)-2-(2-methoxy-phenyl)-5,5-dimethyl-thiazolidin-4-one;
2-(2-Methoxy-phenyl)-5,5-dimethyl-3-naphthalen-2-ylmethyl-thiazolidin-4-one;
3-Benzyl-2-(2-ethoxy-phenyl)-5,5-dimethyl-thiazolidin-4-one;
2-(2-Ethoxy-phenyl)-5,5-dimethyl-3-naphthalen-2-ylmethyl-thiazolidin-4-one;
3-Benzyl-2-(2,6-dimethoxy-phenyl)-5,5-dimethyl-thiazolidin-4-one;
2-(2,6-Dimethoxy-phenyl)-5,5-dimethyl-3-(4-trifluoromethyl-benzyl)-thiazolidin-4-one;
2-(2,6-Dimethoxy-phenyl)-3-(4-ethoxy-benzyl)-5,5-dimethyl-thiazolidin-4-one;
2-(2,6-Dimethoxy-phenyl)-5,5-dimethyl-3-naphthalen-2-ylmethyl-thiazolidin-4-one;
3-(4-Ethoxy-benzyl)-2-(2-methoxy-6-methyl-phenyl)-thiazolidin-4-one;
2-(2,6-Diethoxy-phenyl)-3-(4-ethoxy-benzyl)-thiazolidin-4-one;
2-(2-Chloro-6-methoxy-phenyl)-3-(4-ethoxy-benzyl)-thiazolidin-4-one;
2-(2-Methoxy-6-methyl-phenyl)-3-(4-trifluoromethoxy-benzyl)-thiazolidin-4-one;
2-(2,6-Diethoxy-phenyl)-3-(4-trifluoromethoxy-benzyl)-thiazolidin-4-one;
2-(2-Chloro-6-methoxy-phenyl)-3-(4-trifluoromethoxy-benzyl)-thiazolidin-4-one;
3-(4-Benzyloxy-benzyl)-2-(2,6-dimethoxy-phenyl)-thiazolidin-4-one;
2-(2,6-Dimethoxy-phenyl)-3-[4-(2-hydroxy-ethoxy)-benzyl]-thiazolidin-4-one;
2-(2,6-Dimethoxy-phenyl)-3-[4-(2-methoxy-ethoxy)-benzyl]-thiazolidin-4-one;
2-(2-Methoxy-phenyl)-5-methyl-3-(4-trifluoromethoxy-benzyl)-thiazolidin-4-one;
2-(2-Ethoxy-phenyl)-5-methyl-3-(4-trifluoromethoxy-benzyl)-thiazolidin-4-one;
2-(2,6-Dimethoxy-phenyl)-5,5-dimethyl-3-(4-trifluoromethoxy-benzyl)-thiazolidin-4-one;
3-Benzyl-2-(5-bromo-benzo[1,3]dioxol-4-yl)-thiazolidin-4-one;
3-Benzyl-2-(2-methoxy-naphthalen-1-yl)-thiazolidin-4-one;
3-Benzyl-2-(2,2-dimethyl-2,3-dihydro-benzofuran-7-yl)-thiazolidin-4-one;
3-Benzyl-2-(2,3-dihydro-benzo[1,4]dioxin-5-yl)-thiazolidin-4-one;
2-Benzo[1,3]dioxol-4-yl-3-benzyl-thiazolidin-4-one;
3-Benzyl-2-(2-methoxy-5-methyl-phenyl)-thiazolidin-4-one;
3-Benzyl-2-(5-benzyl-2-methoxy-phenyl)-thiazolidin-4-one;
2-(2,5-Dimethoxy-phenyl)-3-(4-trifluoromethoxy-benzyl)-thiazolidin-4-one;
2-(5-Bromo-benzo[1,3]dioxol-4-yl)-3-(4-trifluoromethoxy-benzyl)-thiazolidin-4-one;
2-(2-Methoxy-naphthalen-1-yl)-3-(4-trifluoromethoxy-benzyl)-thiazolidin-4-one;
2-(4-Hydroxy-2-methoxy-phenyl)-3-(4-trifluoromethoxy-benzyl)-thiazolidin-4-one;
2-Benzo[1,3]dioxol-4-yl-3-(4-trifluoromethoxy-benzyl)-thiazolidin-4-one;
2-(2-Methoxy-5-methyl-phenyl)-3-(4-trifluoromethoxy-benzyl)-thiazolidin-4-one;
2-(6-Methoxy-indan-5-yl)-3-(4-trifluoromethoxy-benzyl)-thiazolidin-4-one;
3-Biphenyl-4-ylmethyl-2-(2,6-dimethoxy-phenyl)-thiazolidin-4-one;
2-(6-Methoxy-quinolin-5-yl)-3-(4-trifluoromethoxy-benzyl)-thiazolidin-4-one;
2-(2,6-Dimethoxy-phenyl)-3-(6-ethoxy-pyridin-3-ylmethyl)-thiazolidin-4-one;
2-(2,6-Dimethoxy-phenyl)-3-(5-ethoxy-pyridin-2-ylmethyl)-thiazolidin-4-one;
2-(2,6-Dimethoxy-phenyl)-3-(6-ethoxy-pyridazin-3-ylmethyl)-thiazolidin-4-one;
2-(2,3-Dihydro-benzo[1,4]dioxin-5-yl)-3-(4-trifluoromethoxy-benzyl)-thiazolidin-4-one;
3-Benzyl-2-(1H-indol-2-yl)-thiazolidin-4-one;
2-(2-Ethoxy-pyridin-3-yl)-3-(4-trifluoromethoxy-benzyl)-thiazolidin-4-one;
2-(1-Methyl-1H-indazol-3-yl)-3-(4-trifluoromethoxy-benzyl)-thiazolidin-4-one;
2-(6-Methoxy-pyridin-2-yl)-3-(4-trifluoromethoxy-benzyl)-thiazolidin-4-one;
3-(4-Ethoxy-benzyl)-2-(2-ethoxy-pyridin-3-yl)-thiazolidin-4-one;
2-(2-Methoxy-pyridin-3-yl)-3-(4-trifluoromethoxy-benzyl)-thiazolidin-4-on;
2-(4,6-Dimethoxy-pyrimidin-5-yl)-3-(4-ethoxy-benzyl)-thiazolidin-4-one;
3-Benzyl-2-(6-methoxy-pyridin-2-yl)-thiazolidin-4-one;
2-(4,6-Dimethoxy-pyrimidin-5-yl)-3-(4-trifluoromethoxy-benzyl)-thiazolidin-4-one;
3-Naphthalen-2-ylmethyl-2,3-dihydro-[2,2]bithiazolyl-4-one;
3-Benzyl-2-(2-methoxy-phenyl)-thiazolidin-4-one;
3-Benzyl-2-(1-methyl-1H-indol-3-yl)-thiazolidin-4-one;
2-(2,6-Dimethoxy-phenyl)-3-thiophen-2-ylmethyl-thiazolidin-4-one;
3-Thiophen-2-ylmethyl-2-(2-trifluoromethyl-phenyl)-thiazolidin-4-one;
2-(2,6-Dimethoxy-phenyl)-5-methyl-3-quinoxalin-2-ylmethyl-thiazolidin-4-one;
2-(2,6-Dimethoxy-phenyl)-3-quinoxalin-2-ylmethyl-thiazolidin-4-one;
3-Benzo[b]thiophen-6-ylmethyl-2-(2,6-dimethoxy-phenyl)-5-methyl-thiazolidin-4-one;
2-(2,6-Dimethoxy-phenyl)-5-methyl-3-(1-methyl-1H-indol-2-ylmethyl)-thiazolidin-4-one;
2-(2,6-Dimethoxy-phenyl)-5-methyl-3-(1-methyl-1H-indol-6-ylmethyl)-thiazolidin-4-one;
3-Benzo[b]thiophen-5-ylmethyl-2-(2,6-dimethoxy-phenyl)-thiazolidin-4-one;
2-(2,6-Dimethoxy-phenyl)-5-methyl-3-(1-methyl-1H-benzoimidazol-2-ylmethyl)-thiazolidin-4-one;
2-(2,6-Dimethoxy-phenyl)-5-methyl-3-quinolin-6-ylmethyl-thiazolidin-4-one;
3-Benzo[b]thiophen-2-ylmethyl-2-(2,6-dimethoxy-phenyl)-5-methyl-thiazolidin-4-one;
3-Benzo[b]thiophen-3-ylmethyl-2-(2,6-dimethoxy-phenyl)-5-methyl-thiazolidin-4-one;
2-(2,6-Dimethoxy-phenyl)-3-(1H-indol-2-ylmethyl)-5-methyl-thiazolidin-4-one;
2-(2,6-Dimethoxy-phenyl)-5-methyl-3-quinolin-7-ylmethyl-thiazolidin-4-one;
3-Benzothiazol-2-ylmethyl-2-(2,6-dimethoxy-phenyl)-5-methyl-thiazolidin-4-one;
2-(2,6-Dimethoxy-phenyl)-5-methyl-3-quinoxalin-6-ylmethyl-thiazolidin-4-one;
3-Benzofuran-2-ylmethyl-2-(2,6-dimethoxy-phenyl)-5-methyl-thiazolidin-4-one;
2-(2,6-Dimethoxy-phenyl)-3-(1H-indol-3-ylmethyl)-5-methyl-thiazolidin-4-one;
2-(2,6-Dimethoxy-phenyl)-3-quinoxalin-6-ylmethyl-thiazolidin-4-one;
2-(3-Fluoro-2,6-dimethoxy-phenyl)-5-methyl-3-(4-trifluoromethoxy-benzyl)-thiazolidin-4-one;
2-(2,6-Dimethoxy-phenyl)-3-(1-methyl-1H-indol-2-ylmethyl)-thiazolidin-4-one;
3-Benzo[b]thiophen-2-ylmethyl-2-(2,6-dimethoxy-phenyl)-thiazolidin-4-one;
2-(2,6-Dimethoxy-phenyl)-3-(1-methyl-1H-indol-6-ylmethyl)-thiazolidin-4-one;
3-(4-Trifluoromethoxy-benzyl)-2-(2,4,6-trimethoxy-phenyl)-thiazolidin-4-one;
2-(3-Fluoro-2,6-dimethoxy-phenyl)-3-(4-trifluoromethoxy-benzyl)-thiazolidin-4-one;
5-Methyl-3-(4-trifluoromethoxy-benzyl)-2-(2,3,6-trimethoxy-phenyl)-thiazolidin-4-one;
2-(2,6-Dimethoxy-3-methyl-phenyl)-5-methyl-3-(4-trifluoromethoxy-benzyl)-thiazolidin-4-one;
3-Benzo[b]thiophen-3-ylmethyl-2-(2,6-dimethoxy-phenyl)-thiazolidin-4-one;
5-Methyl-3-(4-trifluoromethoxy-benzyl)-2-(2,4,6-trimethoxy-phenyl)-thiazolidin-4-one;
2-(2,6-Dimethoxy-3-methyl-phenyl)-3-(4-trifluoromethoxy-benzyl)-thiazolidin-4-one;
2-(2,6-Dimethoxy-phenyl)-3-quinolin-7-ylmethyl-thiazolidin-4-one;
2-(2,6-Dimethoxy-phenyl)-3-(1H-indol-2-ylmethyl)-thiazolidin-4-one;
2-(2,6-Dimethoxy-phenyl)-3-(1-methyl-1H-benzoimidazol-2-ylmethyl)-thiazolidin-4-one;
3-(1H-Benzoimidazol-2-ylmethyl)-2-(4-benzyloxy-3-methoxy-phenyl)-thiazolidin-4-one;
2-(2,6-Dimethoxy-phenyl)-3-quinolin-6-ylmethyl-thiazolidin-4-one;
3-Benzothiazol-2-ylmethyl-2-(2,6-dimethoxy-phenyl)-thiazolidin-4-one;
2-(3-Chloro-2,6-dimethoxy-phenyl)-5-methyl-3-(4-trifluoromethoxy-benzyl)-thiazolidin-4-one;
3-(4-Trifluoromethoxy-benzyl)-2-(2,3,6-trimethoxy-phenyl)-thiazolidin-4-one;
3-Benzofuran-2-ylmethyl-2-(2,6-dimethoxy-phenyl)-thiazolidin-4-one;
2-(2,6-Dimethoxy-phenyl)-5-methyl-3-(1-methyl-1H-benzotriazol-5-ylmethyl)-thiazolidin-4-one;
3-(1H-Benzoimidazol-2-ylmethyl)-2-(2,6-dimethoxy-phenyl)-thiazolidin-4-one;
2-(3-Chloro-2,6-dimethoxy-phenyl)-3-(4-trifluoromethoxy-benzyl)-thiazolidin-4-one;
2-(2,6-Dimethoxy-phenyl)-3-(1H-indol-3-ylmethyl)-thiazolidin-4-one;
2-(2-Fluoro-4,6-dimethoxy-phenyl)-3-(4-trifluoromethoxy-benzyl)-thiazolidin-4-one;
3-(1H-Benzoimidazol-2-ylmethyl)-2-(4-benzyloxy-3-methoxy-phenyl)-5-methyl-thiazolidin-4-one;
2-(2-Chloro-4,6-dimethoxy-phenyl)-3-(4-trifluoromethoxy-benzyl)-thiazolidin-4-one;
2-[2-(S)-(2,3-Dihydroxy-propoxy)-6-methoxy-phenyl]-3-(4-trifluoromethoxy-benzyl)-thiazolidin-4-one;
2-[2-(R)-(2,3-Dihydroxy-propoxy)-6-methoxy-phenyl]-3-(4-trifluoromethoxy-benzyl)-thiazolidin-4-one;
2-[2-Methoxy-6-(2-methoxy-ethoxy)-phenyl]-3-(4-trifluoromethoxy-benzyl)-thiazolidin-4-one;
2-[2-(2-Hydroxy-propoxy)-6-methoxy-phenyl]-3-(4-trifluoromethoxy-benzyl)-thiazolidin-4-one;
3-(4-Ethoxy-benzyl)-2-[2-(2-hydroxy-propoxy)-6-methoxy-phenyl]-thiazolidin-4-one;
2-[2-(2-Hydroxy-propoxy)-6-methoxy-phenyl]-3-(4-isopropoxy-benzyl)-thiazolidin-4-one;
2-(2,6-Dimethoxy-phenyl)-3-[4-(6-trifluoromethyl-pyridin-2-yloxy)-benzyl]thiazolidin-4-one; and
2-(2,6-Dimethoxy-phenyl)-1-oxo-3-(4-trifluoromethoxy-benzyl)-thiazolidin-4-one.

9. A compound of formula (II) 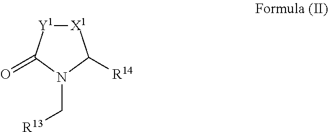
wherein
X¹ represents S or SO₂;
Y¹ represents CH₂ or CH(CH₃);
R¹³ represents a group selected from the group consisting of: 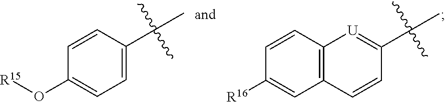
wherein
R¹⁵ represents (C₁₋₄)alkyl or (C₁₋₃)fluoroalkyl; or R¹⁵ represents phenyl or 5- to 6-membered heteroaryl, wherein the phenyl or 5- to 6-membered heteroaryl is independently unsubstituted, or mono-substituted, wherein the substituent is selected from the group consisting of (C₁₋₄)alkyl, and (C₁₋₄)alkoxy;
U represents CH or N;
R¹⁶ represents hydrogen or methoxy;
R¹⁴ represents a group selected from the group consisting of: 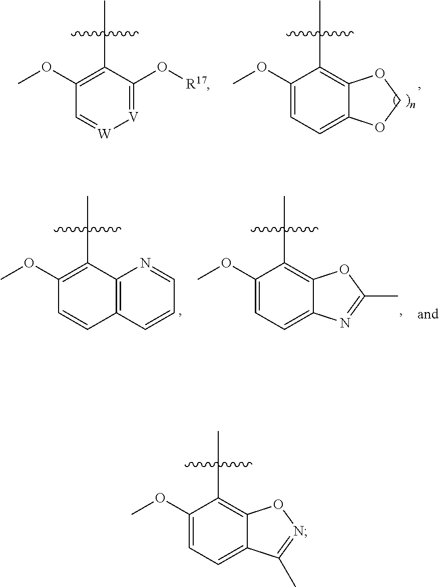
wherein
V represents CH and W represents CR¹⁸ or N; or V represents N and W represents CH;
R¹⁷ represents methyl, ethyl or hydroxyethyl;
R¹⁸ represents hydrogen, methyl or fluoro; and
n represents the integer 1 or 2;
in a free or a pharmaceutically acceptable salt form.

10. The compound according to claim 9; wherein R¹³ represents 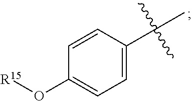
wherein
R¹⁵ represents (C₁₋₄)alkyl or (C₁₋₃)fluoroalkyl; or
R¹⁵ represents phenyl or 5- to 6-membered heteroaryl, wherein the phenyl or 5- to 6-membered heteroaryl is independently unsubstituted, or mono-substituted, wherein the substituent is selected from the group consisting of (C₁₋₄)alkyl, and (C₁₋₄)alkoxy;
in a free or a pharmaceutically acceptable salt form.

11. The compound according to claim 9;
wherein R¹⁴ represents 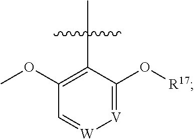
wherein
V represents CH and W represents CR¹⁸ or N; or
V represents N and W represents CH;
R¹⁷ represents methyl, ethyl or hydroxyethyl; and
R¹⁸ represents hydrogen, methyl or fluoro;
in a free or a pharmaceutically acceptable salt form.

12. The compound according to claim 9 selected from the group consisting of:
2-(2,6-Dimethoxy-phenyl)-3-(4-methoxy-benzyl)-thiazolidin-4-one;
2-(2,6-Dimethoxy-phenyl)-3-(4-trifluoromethoxy-benzyl)-thiazolidin-4-one;
3-(4-tert-Butoxy-benzyl)-2-(2,6-dimethoxy-phenyl)-thiazolidin-4-one;
3-(4-Difluoromethoxy-benzyl)-2-(2,6-dimethoxy-phenyl)-thiazolidin-4-one;
2-(2,6-Dimethoxy-phenyl)-3-(4-isopropoxy-benzyl)-thiazolidin-4-one;
2-(2,6-Dimethoxy-phenyl)-3-(4-ethoxy-benzyl)-thiazolidin-4-one;
2-(2,6-Dimethoxy-phenyl)-3-(4-phenoxy-benzyl)-thiazolidin-4-one;
2-(2,6-Dimethoxy-phenyl)-3-naphthalen-2-ylmethyl-thiazolidin-4-one;
3-(4-Ethoxy-benzyl)-2-(6-methoxy-2,3-dihydro-benzo[1,4]dioxin-5-yl)-thiazolidin-4-one;
2-(6-Methoxy-2,3-dihydro-benzo[1,4]dioxin-5-yl)-3-(4-trifluoromethoxy-benzyl)-thiazolidin-4-one;
3-(4-Ethoxy-benzyl)-2-(5-methoxy-benzo[1,3]dioxol-4-yl)-thiazolidin-4-one;
2-(5-Methoxy-benzo[1,3]dioxol-4-yl)-3-(4-trifluoromethoxy-benzyl)-thiazolidin-4-one;
2-(2,6-Dimethoxy-phenyl)-3-(4-ethoxy-benzyl)-5-methyl-thiazolidin-4-one;
2-(2,6-Dimethoxy-phenyl)-5-methyl-3-naphthalen-2-ylmethyl-thiazolidin-4-one;
3-(4-Ethoxy-benzyl)-2-(2-ethoxy-6-methoxy-phenyl)-thiazolidin-4-one;
2-(2-Ethoxy-6-methoxy-phenyl)-3-(4-trifluoromethoxy-benzyl)-thiazolidin-4-one;
2-(2,6-Dimethoxy-phenyl)-5-methyl-3-(4-trifluoromethoxy-benzyl)-thiazolidin-4-one;
2-(2,6-Dimethoxy-phenyl)-3-[4-(2,2,2-trifluoro-ethoxy)-benzyl]-thiazolidin-4-one;
3-(4-Ethoxy-benzyl)-2-(6-methoxy-3-methyl-benzo[d]isoxazol-7-yl)-thiazolidin-4-one;
3-(4-Ethoxy-benzyl)-2-(6-methoxy-2-methyl-benzooxazol-7-yl)-thiazolidin-4-one;
3-(4-Ethoxy-benzyl)-2-(7-methoxy-quinolin-8-yl)-thiazolidin-4-one;
2-(6-Methoxy-3-methyl-benzo[d]isoxazol-7-yl)-3-(4-trifluoromethoxy-benzyl)-thiazolidin-4-one;
2-(6-Methoxy-2-methyl-benzooxazol-7-yl)-3-(4-trifluoromethoxy-benzyl)-thiazolidin-4-one;
2-(7-Methoxy-quinolin-8-yl)-3-(4-trifluoromethoxy-benzyl)-thiazolidin-4-one;
2-(2,4-Dimethoxy-pyridin-3-yl)-3-(4-trifluoromethoxy-benzyl)-thiazolidin-4-one;
2-(2,4-Dimethoxy-pyridin-3-yl)-3-(4-ethoxy-benzyl)-thiazolidin-4-one;
2-(2,6-Dimethoxy-phenyl)-3-quinolin-2-ylmethyl-thiazolidin-4-one;
2-(3,5-Dimethoxy-pyridin-4-yl)-3-(4-trifluoromethoxy-benzyl)-thiazolidin-4-one;
2-(3,5-Dimethoxy-pyridin-4-yl)-3-(4-ethoxy-benzyl)-thiazolidin-4-one;
2-(2,6-Dimethoxy-phenyl)-5-methyl-3-quinolin-2-ylmethyl-thiazolidin-4-one;
2-(2,6-Dimethoxy-phenyl)-3-(6-methoxy-naphthalen-2-ylmethyl)-5-methyl-thiazolidin-4-one;
2-(2,6-Dimethoxy-phenyl)-3-(6-methoxy-naphthalen-2-ylmethyl)-thiazolidin-4-one;
2-(4-Fluoro-2,6-dimethoxy-phenyl)-3-(4-trifluoromethoxy-benzyl)-thiazolidin-4-one;
2-(2,6-Dimethoxy-4-methyl-phenyl)-3-(4-trifluoromethoxy-benzyl)-thiazolidin-4-one;
2-(2,6-Dimethoxy-4-methyl-phenyl)-5-methyl-3-(4-trifluoromethoxy-benzyl)-thiazolidin-4-one;
2-(4-Fluoro-2,6-dimethoxy-phenyl)-5-methyl-3-(4-trifluoromethoxy-benzyl)-thiazolidin-4-one;
2-[2-(2-Hydroxy-ethoxy)-6-methoxy-phenyl]-3-(4-trifluoromethoxy-benzyl)-thiazolidin-4-one;
2-[2-(2-Hydroxy-ethoxy)-6-methoxy-phenyl]-3-(4-trifluoromethoxy-benzyl)-thiazolidin-4-one;
2-[2-(2-Hydroxy-ethoxy)-6-methoxy-phenyl]-3-(4-isopropoxy-benzyl)-thiazolidin-4-on;
2-(2,6-Dimethoxy-phenyl)-3-[4-(pyrimidin-5-yloxy)-benzyl]-thiazolidin-4-one;
2-(2,6-Dimethoxy-phenyl)-3-[4-(pyridin-2-yloxy)-benzyl]-thiazolidin-4-one;
2-(2,6-Dimethoxy-phenyl)-3-[4-(pyridin-3-yloxy)-benzyl]-thiazolidin-4-one;
2-(2,6-Dimethoxy-phenyl)-3-[4-(pyrazin-2-yloxy)-benzyl]-thiazolidin-4-one;
2-(2,6-Dimethoxy-phenyl)-3-[4-(6-methyl-pyridazin-3-yloxy)-benzyl]-thiazolidin-4-one;
2-(2,6-Dimethoxy-phenyl)-3-[4-(5-methyl-pyridin-2-yloxy)-benzyl]-thiazolidin-4-one;
2-(2,6-Dimethoxy-phenyl)-3-[4-(6-methoxy-pyridin-2-yloxy)-benzyl]-thiazolidin-4-one;
2-(2,6-Dimethoxy-phenyl)-3-[4-(6-methyl-pyridin-2-yloxy)-benzyl]-thiazolidin-4-one;
2-(2,6-Dimethoxy-phenyl)-3-[4-(4-methyl-pyridin-2-yloxy)-benzyl]-thiazolidin-4-one;
2-(2,6-Dimethoxy-phenyl)-3-[4-(3-methyl-pyridin-2-yloxy)-benzyl]thiazolidin-4-one;
2-(2,6-Dimethoxy-phenyl)-3-[4-(5-methoxy-pyridin-2-yloxy)-benzyl]-thiazolidin-4-one;
2-(2,6-Dimethoxy-phenyl)-1,1-dioxo-3-(4-trifluoromethoxy-benzyl)-thiazolidin-4-one;
2-(2,6-Dimethoxy-phenyl)-3-(4-isopropoxy-benzyl)-1,1-dioxo-thiazolidin-4-one; and
2-(2,6-Dimethoxy-phenyl)-3-(4-ethoxy-benzyl)-1,1-dioxo-thiazolidin-4-one;
in a free or a pharmaceutically acceptable salt form.

13. The pharmaceutical composition containing, as active principle, the compound according to claim 9, in a free or a pharmaceutically acceptable salt form, and at least one therapeutically inert excipient.

14. A method for the treatment or prophylaxis of a disease or disorder related to the orexin receptor wherein the compound of claim 9 is administered to a subject.

15. The method of claim 14, wherein said disease or disorder is selected from the group consisting of: all types of sleep disorders, of stress related syndromes, of addictions, of cognitive dysfunctions in the healthy population and in psychiatric and neurologic disorders, and eating or drinking disorders.
